SEQUENCE LISTING (206 sequences)

SEQ ID NO: 1 - DNA, 50 bp, synthetic construct
  misc_feature 1..50: note = Forward primer sequence for SARS-CoV-2 virus Spike gene
  source 1..50: mol_type = other DNA
tctaatacctttgctcattgactttttttcttgttttattgccactagtc

SEQ ID NO: 2 - DNA, 58 bp, synthetic construct
  misc_feature 1..58: note = Reverse primer sequence for SARS-COV-2 virus Spike gene
  source 1..58: mol_type = other DNA
tacaattaatacgactcactatagggcatatttttgtcagggtaataaacaccacgtg

SEQ ID NO: 3 - DNA, 50 bp, synthetic construct
  misc_feature 1..50: note = Forward primer sequence for SARS-COV-2 virus Spike gene
  source 1..50: mol_type = other DNA
tctaatacctttgctcattgacacctttcttttccaatgttacttggttc

SEQ ID NO: 4 - DNA, 58 bp, synthetic construct
  misc_feature 1..58: note = Reverse primer sequence for SARS-COV-2 virus Spike gene
  source 1..58: mol_type = other DNA
tacaattaatacgactcactatagggcatattttatgttagacttctcagtggaagca

SEQ ID NO: 5 - DNA, 50 bp, synthetic construct
  misc_feature 1..50: note = Forward primer sequence for SARS-COV-2 virus Spike gene
  source 1..50: mol_type = other DNA
tctaatacctttgctcattgactttcttattgttaataacgctactaatg

SEQ ID NO: 6 - DNA, 58 bp, synthetic construct
  misc_feature 1..58: note = Reverse primer sequence for SARS-COV-2 virus Spike gene
  source 1..58: mol_type = other DNA
tacaattaatacgactcactatagggcatatttcattcgcactagaataaactctgaa

SEQ ID NO: 7 - DNA, 50 bp, synthetic construct
  misc_feature 1..50: note = Forward primer sequence for SARS-COV-2 virus Spike gene
  source 1..50: mol_type = other DNA
tctaatacctttgctcattgacttttaagcacacgcctattaatttagtg

SEQ ID NO: 8 - DNA, 58 bp, synthetic construct
  misc_feature 1..58: note = Reverse primer sequence for SARS-COV-2 virus Spike gene
  source 1..58: mol_type = other DNA
tacaattaatacgactcactatagggcatatttccacataataagctgcagcaccagc

SEQ ID NO: 9 - DNA, 47 bp, synthetic construct
  misc_feature 1..47: note = Forward primer sequence for SARS-COV-2 virus Spike gene
  source 1..47: mol_type = other DNA
tctaatacctttgctcattgactgtaattagaggtgatgaagtcaga

SEQ ID NO: 10 - DNA, 58 bp, synthetic construct
  misc_feature 1..58: note = Reverse primer sequence for SARS-COV-2 virus Spike gene
  source 1..58: mol_type = other DNA
tacaattaatacgactcactatagggcatatttaaaggtttgagattagacttcctaa

SEQ ID NO: 11 - DNA, 50 bp, synthetic construct
  misc_feature 1..50: note = Forward primer sequence for SARS-COV-2 virus Spike gene
  source 1..50: mol_type = other DNA
tctaatacctttgctcattgacttttatttcaactgaaatytatcaggcc

SEQ ID NO: 12 - DNA, 58 bp, synthetic construct
  misc_feature 1..58: note = Reverse primer sequence for SARS-COV-2 virus Spike gene
  source 1..58: mol_type = other DNA
tacaattaatacgactcactatagggcatatttaaagtactactactctgtatggttg

SEQ ID NO: 13 - DNA, 50 bp, synthetic construct
  misc_feature 1..50: note = Forward primer sequence for SARS-COV-2 virus Spike gene
  source 1..50: mol_type = other DNA
tctaatacctttgctcattgactttagtgttataacaccaggaacaaata

SEQ ID NO: 14 - DNA, 58 bp, synthetic construct
  misc_feature 1..58: note = Reverse primer sequence for SARS-COV-2 virus Spike gene
  source 1..58: mol_type = other DNA
tacaattaatacgactcactatagggcatattttgcatgaatagcaacagggacttct

SEQ ID NO: 15 - DNA, 50 bp, synthetic construct
  misc_feature 1..50: note = Forward primer sequence for SARS-COV-2 virus Spike gene
  source 1..50: mol_type = other DNA
tctaatacctttgctcattgacttttgcaggtatatgcgctagttatcag

SEQ ID NO: 16 - DNA, 58 bp, synthetic construct
  misc_feature 1..58: note = Reverse primer sequence for SARS-COV-2 virus Spike gene
  source 1..58: mol_type = other DNA
tacaattaatacgactcactatagggcatattttggtatggcaatagagttattagag

SEQ ID NO: 17 - DNA, 30 bp, synthetic construct
  misc_feature 1..30: note = Hybridization probe sequence for SARS-COV-2 virus detection
  source 1..30: mol_type = other DNA
tttttctagtctctaktcagtgtgtttttt

SEQ ID NO: 18 - DNA, 30 bp, synthetic construct
  misc_feature 1..30: note = Hybridization probe sequence for SARS-COV-2 virus detection
  source 1..30: mol_type = other DNA
tttttttgtctctagtcagtgttttttttt

SEQ ID NO: 19 - DNA, 30 bp, synthetic construct
  misc_feature 1..30: note = Hybridization probe sequence for SARS-COV-2 virus detection
  source 1..30: mol_type = other DNA
tttttttagtctctattcagtgtttttttt

SEQ ID NO: 20 - DNA, 30 bp, synthetic construct
  misc_feature 1..30: note = Hybridization probe sequence for SARS-COV-2 virus detection
  source 1..30: mol_type = other DNA
ttttttaatyttacaamcagaactcttttt

SEQ ID NO: 21 - DNA, 30 bp, synthetic construct
  misc_feature 1..30: note = Hybridization probe sequence for SARS-COV-2 virus detection
  source 1..30: mol_type = other DNA
tttttttatcttacaaccagaacctttttt

SEQ ID NO: 22 - DNA, 30 bp, synthetic construct
  misc_feature 1..30: note = Hybridization probe sequence for SARS-COV-2 virus detection
  source 1..30: mol_type = other DNA
tttttttatcttacaaccagaacttttttt

SEQ ID NO: 23 - DNA, 32 bp, synthetic construct
  misc_feature 1..32: note = Hybridization probe sequence for SARS-COV-2 virus detection
  source 1..32: mol_type = other DNA
tttttcccatgctatacatgtctctgtttttt

SEQ ID NO: 24 - DNA, 32 bp, synthetic construct
  misc_feature 1..32: note = Hybridization probe sequence for SARS-COV-2 virus detection
  source 1..32: mol_type = other DNA
tttttttttccatgctatctctgggatttttt

SEQ ID NO: 25 - DNA, 32 bp, synthetic construct
  misc_feature 1..32: note = Hybridization probe sequence for SARS-COV-2 virus detection
  source 1..32: mol_type = other DNA
tttttcagaggtttgmtaaccctgtctttttt

SEQ ID NO: 26 - DNA, 30 bp, synthetic construct
  misc_feature 1..30: note = Hybridization probe sequence for SARS-COV-2 virus detection
  source 1..30: mol_type = other DNA
tttttttggtttgataaccctgcttttttt

SEQ ID NO: 27 - DNA, 30 bp, synthetic construct
  misc_feature 1..30: note = Hybridization probe sequence for SARS-COV-2 virus detection
  source 1..30: mol_type = other DNA
tttttttggtttgctaaccctgcttttttt

SEQ ID NO: 28 - DNA, 30 bp, synthetic construct
  misc_feature 1..30: note = Hybridization probe sequence for SARS-COV-2 virus detection
  source 1..30: mol_type = other DNA
ttttattttgtaatkatccatttttgtttt

SEQ ID NO: 29 - DNA, 30 bp, synthetic construct
  misc_feature 1..30: note = Hybridization probe sequence for SARS-COV-2 virus detection
  source 1..30: mol_type = other DNA
tttttctttgtaatgatccattttcttttt

SEQ ID NO: 30 - DNA, 30 bp, synthetic construct
  misc_feature 1..30: note = Hybridization probe sequence for SARS-COV-2 virus detection
  source 1..30: mol_type = other DNA
tttttttttgtaattatccattttcttttt

SEQ ID NO: 31 - DNA, 30 bp, synthetic construct
  misc_feature 1..30: note = Hybridization probe sequence for SARS-COV-2 virus detection
  source 1..30: mol_type = other DNA
tttttagttgkatggaaagtgagttctttt

SEQ ID NO: 32 - DNA, 30 bp, synthetic construct
  misc_feature 1..30: note = Hybridization probe sequence for SARS-COV-2 virus detection
  source 1..30: mol_type = other DNA
tttctctaaaagttggatggaaactcttct

SEQ ID NO: 33 - DNA, 30 bp, synthetic construct
  misc_feature 1..30: note = Hybridization probe sequence for SARS-COV-2 virus detection
  source 1..30: mol_type = other DNA
tttcttcaaagttgtatggaaagccttctt

SEQ ID NO: 34 - DNA, 32 bp, synthetic construct
  misc_feature 1..32: note = Hybridization probe sequence for SARS-COV-2 virus detection
  source 1..32: mol_type = other DNA
ttttttttcaaactttacttgctttactcttt

SEQ ID NO: 35 - DNA, 32 bp, synthetic construct
  misc_feature 1..32: note = Hybridization probe sequence for SARS-COV-2 virus detection
  source 1..32: mol_type = other DNA
ttttttttcaaactttacatagaagccttttt

SEQ ID NO: 36 - DNA, 32 bp, synthetic construct
  misc_feature 1..32: note = Hybridization probe sequence for SARS-COV-2 virus detection
  source 1..32: mol_type = other DNA
ttttctacatagaagttatttgactccctttt

SEQ ID NO: 37 - DNA, 32 bp, synthetic construct; identical to SEQ ID NO: 36
  misc_feature 1..32: note = Hybridization probe sequence for SARS-COV-2 virus detection
  source 1..32: mol_type = other DNA

SEQ ID NO: 38 - DNA, 30 bp, synthetic construct
  misc_feature 1..30: note = Hybridization probe sequence for SARS-COV-2 virus detection
  source 1..30: mol_type = other DNA
tttctactcctggtgrttcttcttcatttt

SEQ ID NO: 39 - DNA, 30 bp, synthetic construct
  misc_feature 1..30: note = Hybridization probe sequence for SARS-COV-2 virus detection
  source 1..30: mol_type = other DNA
ttttttccctggtgattcttctttcttttt

SEQ ID NO: 40 - DNA, 30 bp, synthetic construct
  misc_feature 1..30: note = Hybridization probe sequence for SARS-COV-2 virus detection
  source 1..30: mol_type = other DNA
ttttttccctggtggttcttcttttttttt

SEQ ID NO: 41 - DNA, 32 bp, synthetic construct
  misc_feature 1..32: note = Hybridization probe sequence for SARS-COV-2 virus detection
  source 1..32: mol_type = other DNA
tttttaattctaamaakcttgattctaatttt

SEQ ID NO: 42 - DNA, 30 bp, synthetic construct
  misc_feature 1..30: note = Hybridization probe sequence for SARS-COV-2 virus detection
  source 1..30: mol_type = other DNA
tttttaattctaacaatcttgatttctttt

SEQ ID NO: 43 - DNA, 30 bp, synthetic construct
  misc_feature 1..30: note = Hybridization probe sequence for SARS-COV-2 virus detection
  source 1..30: mol_type = other DNA
ttttttattctaacaagcttgatttttttt

SEQ ID NO: 44 - DNA, 30 bp, synthetic construct
  misc_feature 1..30: note = Hybridization probe sequence for SARS-COV-2 virus detection
  source 1..30: mol_type = other DNA
ttttctattctaaaaatcttgatttctttt

SEQ ID NO: 45 - DNA, 30 bp, synthetic construct
  misc_feature 1..30: note = Hybridization probe sequence for SARS-COV-2 virus detection
  source 1..30: mol_type = other DNA
tttctataattacctgtatagattgtcttt

SEQ ID NO: 46 - DNA, 30 bp, synthetic construct
  misc_feature 1..30: note = Hybridization probe sequence for SARS-COV-2 virus detection
  source 1..30: mol_type = other DNA
tttttttaattacctgtatagatttctttt

SEQ ID NO: 47 - DNA, 30 bp, synthetic construct
  misc_feature 1..30: note = Hybridization probe sequence for SARS-COV-2 virus detection
  source 1..30: mol_type = other DNA
tttttcataattactggtatagatcttttt

SEQ ID NO: 48 - DNA, 30 bp, synthetic construct
  misc_feature 1..30: note = Probe sequence for SARS-COV-2 virus detection
  source 1..30: mol_type = other DNA
ttttttcgccggtagcacacctcttttttt

SEQ ID NO: 49 - DNA, 30 bp, synthetic construct
  misc_feature 1..30: note = Hybridization probe sequence for SARS-COV-2 virus detection
  source 1..30: mol_type = other DNA
ttttcttccggtaacacacctttttttttt

SEQ ID NO: 50 - DNA, 32 bp, synthetic construct
  misc_feature 1..32: note = Hybridization probe sequence for SARS-COV-2 virus detection
  source 1..32: mol_type = other DNA
ttttttaatggtgttraaggttttaatttttt

SEQ ID NO: 51 - DNA, 30 bp, synthetic construct
  misc_feature 1..30: note = Hybridization probe sequence for SARS-COV-2 virus detection
  source 1..30: mol_type = other DNA
ttttttctggtgttgaaggttttacttttt

SEQ ID NO: 52 - DNA, 30 bp, synthetic construct
  misc_feature 1..30: note = Hybridization probe sequence for SARS-COV-2 virus detection
  source 1..30: mol_type = other DNA
tttttttatggtgttaaaggttttcttttt

SEQ ID NO: 53 - DNA, 30 bp, synthetic construct
  misc_feature 1..30: note = Hybridization probe sequence for SARS-COV-2 virus detection
  source 1..30: mol_type = other DNA
tttttttatggtgctgaaggttcttttttt

SEQ ID NO: 54 - DNA, 33 bp, synthetic construct
  misc_feature 1..33: note = Hybridization probe sequence for SARS-COV-2 virus detection
  source 1..33: mol_type = other DNA
ttttttttccaacccactwatggtgtttttttt

SEQ ID NO: 55 - DNA, 30 bp, synthetic construct
  misc_feature 1..30: note = Hybridization probe sequence for SARS-COV-2 virus detection
  source 1..30: mol_type = other DNA
ttttttttacccactaatggtgtctttttt

SEQ ID NO: 56 - DNA, 30 bp, synthetic construct
  misc_feature 1..30: note = Hybridization probe sequence for SARS-COV-2 virus detection
  source 1..30: mol_type = other DNA
ttttttttacccacttatggtgtctttttt

SEQ ID NO: 57 - DNA, 32 bp, synthetic construct
  misc_feature 1..32: note = Hybridization probe sequence for SARS-COV-2 virus detection
  source 1..32: mol_type = other DNA
ttttttatcagactcmgactaattctcttttt

SEQ ID NO: 58 - DNA, 30 bp, synthetic construct
  misc_feature 1..30: note = Hybridization probe sequence for SARS-COV-2 virus detection
  source 1..30: mol_type = other DNA
ttttttccagactcagactaatttcttttt

SEQ ID NO: 59 - DNA, 30 bp, synthetic construct
  misc_feature 1..30: note = Hybridization probe sequence for SARS-COV-2 virus detection
  source 1..30: mol_type = other DNA
tttttcttcagactccgactaatctttttt

SEQ ID NO: 60 - DNA, 30 bp, synthetic construct
  misc_feature 1..30: note = Hybridization probe sequence for SARS-COV-2 virus detection
  source 1..30: mol_type = other DNA
ttttttccagactcatactaatttcttttt

SEQ ID NO: 61 - DNA, 30 bp, synthetic construct
  misc_feature 1..30: note = Hybridization probe sequence for SARS-COV-2 virus detection
  source 1..30: mol_type = other DNA
ttttttccagactcacactaatttcttttt

SEQ ID NO: 62 - DNA, 30 bp, synthetic construct
  misc_feature 1..30: note = Hybridization probe sequence for SARS-COV-2 virus detection
  source 1..30: mol_type = other DNA
ttttttcagactaattctcmtcggcttttt

SEQ ID NO: 63 - DNA, 30 bp, synthetic construct
  misc_feature 1..30: note = Hybridization probe sequence for SARS-COV-2 virus detection
  source 1..30: mol_type = other DNA
tttttttctaattctcctcggcgttttttt

SEQ ID NO: 64 - DNA, 30 bp, synthetic construct
  misc_feature 1..30: note = Hybridization probe sequence for SARS-COV-2 virus detection
  source 1..30: mol_type = other DNA
tttttttttaattctcatcggcgttttttt

SEQ ID NO: 65 - DNA, 30 bp, synthetic construct
  misc_feature 1..30: note = Hybridization probe sequence for SARS-COV-2 virus detection
  source 1..30: mol_type = other DNA
ttttcacttggtgyagaaaattcagttttt

SEQ ID NO: 66 - DNA, 30 bp, synthetic construct
  misc_feature 1..30: note = Hybridization probe sequence for SARS-COV-2 virus detection
  source 1..30: mol_type = other DNA
tcttcttcttggtgcagaaaattattcttt

SEQ ID NO: 67 - DNA, 30 bp, synthetic construct
  misc_feature 1..30: note = Hybridization probe sequence for SARS-COV-2 virus detection
  source 1..30: mol_type = other DNA
tcttcttcttggtgtagaaaattattcttt

SEQ ID NO: 68 - DNA, 28 bp, synthetic construct
  misc_feature 1..28: note = T7 promoter sequence on each forward primer
  source 1..28: mol_type = other DNA
ttttctaatacctttgctcattgacttt

SEQ ID NO: 69 - DNA, 19 bp, synthetic construct
  misc_feature 1..19: note = Detector probe binding nucleotide sequence complementary to to fluorescent labeled detect probe sequence on each reverse primer
  source 1..19: mol_type = other DNA
taatacgactcactatagg

SEQ ID NO: 70-71 - skipped (2 entries)

SEQ ID NO: 72 - DNA, 50 bp, synthetic construct
  misc_feature 1..50: note = Forward primer sequence for SARS-COV-2 virus
  source 1..50: mol_type = other DNA
ttttaatcggtgctcttgaccaaattgcaatcttgattctaaggttggtg

SEQ ID NO: 73 - DNA, 55 bp, synthetic construct
  misc_feature 1..55: note = Reverse primer sequence for SARS-COV-2 virus
  source 1..55: mol_type = other DNA
gaatttaatacgactcactatagggataataaagtactactactctgtatggttg

SEQ ID NO: 74 - DNA, 29 bp, synthetic construct
  misc_feature 1..29: note = Hybridization probe sequence for SARS-COV-2 virus detection
  source 1..29: mol_type = other DNA
tttctataattaccdgtatagattgcttt

SEQ ID NO: 75 - DNA, 30 bp, synthetic construct; identical to SEQ ID NO: 46
  misc_feature 1..30: note = Hybridization probe sequence for SARS-COV-2 virus detection
  source 1..30: mol_type = other DNA

SEQ ID NO: 76 - DNA, 30 bp, synthetic construct
  misc_feature 1..30: note = Hybridization probe sequence for SARS-COV-2 virus detection
  source 1..30: mol_type = other DNA
tttttcataattaccggtatagatcttttt

SEQ ID NO: 77 - DNA, 30 bp, synthetic construct
  misc_feature 1..30: note = Hybridization probe sequence for SARS-COV-2 virus detection
  source 1..30: mol_type = other DNA
ttttttttaattaccagtatagactttttt

SEQ ID NO: 78 - DNA, 30 bp, synthetic construct
  misc_feature 1..30: note = Hybridization probe sequence for SARS-COV-2 virus detection
  source 1..30: mol_type = other DNA
tttttcgccggtarcamaccttgtattttt

SEQ ID NO: 79 - DNA, 30 bp, synthetic construct
  misc_feature 1..30: note = Hybridization probe sequence for SARS-COV-2 virus detection
  source 1..30: mol_type = other DNA
tttttcggtagcamaccttgtaatgttttt

SEQ ID NO: 80 - DNA, 30 bp, synthetic construct
  misc_feature 1..30: note = Hybridization probe sequence for SARS-COV-2 virus detection
  source 1..30: mol_type = other DNA
tttttttggtagcacaccttgttttttttt

SEQ ID NO: 81 - DNA, 30 bp, synthetic construct
  misc_feature 1..30: note = Hybridization probe sequence for SARS-COV-2 virus detection
  source 1..30: mol_type = other DNA
tttttttgtagcaaaccttgtatttttttt

SEQ ID NO: 82 - DNA, 27 bp, synthetic construct
  misc_feature 1..27: note = Hybridization probe sequence for SARS-COV-2 virus detection
  source 1..27: mol_type = other DNA
taatggtgytrmaggttttaatttttt

SEQ ID NO: 83 - DNA, 30 bp, synthetic construct; identical to SEQ ID NO: 51
  misc_feature 1..30: note = Hybridization probe sequence for SARS-COV-2 virus detection
  source 1..30: mol_type = other DNA

SEQ ID NO: 84 - DNA, 30 bp, synthetic construct; identical to SEQ ID NO: 52
  misc_feature 1..30: note = Hybridization probe sequence for SARS-COV-2 virus detection
  source 1..30: mol_type = other DNA

SEQ ID NO: 85 - DNA, 30 bp, synthetic construct; identical to SEQ ID NO: 53
  misc_feature 1..30: note = Hybridization probe sequence for SARS-COV-2 virus detection
  source 1..30: mol_type = other DNA

SEQ ID NO: 86 - DNA, 30 bp, synthetic construct
  misc_feature 1..30: note = Hybridization probe sequence for SARS-COV-2 virus detection
  source 1..30: mol_type = other DNA
ttttttcggtgttgcaggttttatcttttt

SEQ ID NO: 87 - DNA, 32 bp, synthetic construct
  misc_feature 1..32: note = Hybridization probe sequence for SARS-COV-2 virus detection
  source 1..32: mol_type = other DNA
ttttctaattgttactytcctttacaattttt

SEQ ID NO: 88 - DNA, 30 bp, synthetic construct
  misc_feature 1..30: note = Hybridization probe sequence for SARS-COV-2 virus detection
  source 1..30: mol_type = other DNA
tttttttttgttactttcctttactttttt

SEQ ID NO: 89 - DNA, 30 bp, synthetic construct
  misc_feature 1..30: note = Hybridization probe sequence for SARS-COV-2 virus detection
  source 1..30: mol_type = other DNA
tttttttttgttactctcctttactttttt

SEQ ID NO: 90 - DNA, 26 bp, synthetic construct
  misc_feature 1..26: note = Hybridization probe sequence for SARS-COV-2 virus detection
  source 1..26: mol_type = other DNA
tcctttacaaycatatggtttttttt

SEQ ID NO: 91 - DNA, 24 bp, synthetic construct
  misc_feature 1..24: note = Hybridization probe sequence for SARS-COV-2 virus detection
  source 1..24: mol_type = other DNA
ttacgatcatatagtttccttttt

SEQ ID NO: 92 - DNA, 23 bp, synthetic construct
  misc_feature 1..23: note = Hybridization probe sequence for SARS-COV-2 virus detection
  source 1..23: mol_type = other DNA
ctttacaatcatatggtcttttt

SEQ ID NO: 93 - DNA, 30 bp, synthetic construct
  misc_feature 1..30: note = Hybridization probe sequence for SARS-COV-2 virus detection
  source 1..30: mol_type = other DNA
tttttctctttacaaccatatggtcttttt

SEQ ID NO: 94 - DNA, 30 bp, synthetic construct
  misc_feature 1..30: note = Hybridization probe sequence for SARS-COV-2 virus detection
  source 1..30: mol_type = other DNA
tttttcttacgatcatatagtttctttttt

SEQ ID NO: 95 - DNA, 33 bp, synthetic construct; identical to SEQ ID NO: 54
  misc_feature 1..33: note = Hybridization probe sequence for SARS-COV-2 virus detection
  source 1..33: mol_type = other DNA

SEQ ID NO: 96 - DNA, 30 bp, synthetic construct; identical to SEQ ID NO: 55
  misc_feature 1..30: note = Hybridization probe sequence for SARS-COV-2 virus detection
  source 1..30: mol_type = other DNA

SEQ ID NO: 97 - DNA, 30 bp, synthetic construct; identical to SEQ ID NO: 56
  misc_feature 1..30: note = Hybridization probe sequence for SARS-COV-2 virus detection
  source 1..30: mol_type = other DNA

SEQ ID NO: 98 - DNA, 27 bp, synthetic construct
  misc_feature 1..27: note = Detector probe sequence for SARS-COV-2 virus detection
  modified_base 1: mod_base = OTHER; note = Thymidine at position 1 is biotinylated
  source 1..27: mol_type = other DNA
ttttaatcggtgctcttgaccaaattg

SEQ ID NO: 99 - DNA, 29 bp, synthetic construct
  misc_feature 1..29: note = Detector probe sequence for SARS-COV-2 virus detection
  modified_base 1: mod_base = OTHER; note = Thymidine at position 1 is biotinylated
  source 1..29: mol_type = other DNA
ttttttgagagagatatttcaactgattt

SEQ ID NO: 100 - DNA, 21 bp, synthetic construct
  misc_feature 1..21: note = Detector probe sequence for SARS-COV-2 virus detection
  modified_base 1: mod_base = OTHER; note = Thymidine at position 1 is modified with a fluorescent label
  source 1..21: mol_type = other DNA
tttctaccgtactctagcttt

SEQ ID NO: 101 - DNA, 21 bp, synthetic construct
  misc_feature 1..21: note = Detector probe sequence for SARS-COV-2 virus detection
  modified_base 1: mod_base = OTHER; note = Thymidine at position 1 is modified with a fluorescent label
  source 1..21: mol_type = other DNA
tttctgttgagttatcccttt

SEQ ID NO: 102 - DNA, 43 bp, synthetic construct
  misc_feature 1..43: note = Forward primer sequence for Influenza A virus
  source 1..43: mol_type = other DNA
tttctaccgtactctagctacttctaaccgaggtcgaaacgta

SEQ ID NO: 103 - DNA, 60 bp, synthetic construct
  misc_feature 1..60: note = Reverse primer sequence for Influenza A virus
  source 1..60: mol_type = other DNA
gaatttaatacgactcactatagggataactcaacaggcattytggacaaakcgtctacg

SEQ ID NO: 104 - DNA, 34 bp, synthetic construct
  misc_feature 1..34: note = Hybridization probe sequence for Influenza A virus detection
  source 1..34: mol_type = other DNA
tttttcgtgcccagtgagcgaggactgcattttt

SEQ ID NO: 105 - DNA, 42 bp, synthetic construct
  misc_feature 1..42: note = Forward primer sequence for Influenza B virus
  source 1..42: mol_type = other DNA
tttctaccgtactctagctatcctcaactcactcttcgagcg

SEQ ID NO: 106 - DNA, 60 bp, synthetic construct
  misc_feature 1..60: note = Reverse primer sequence for Influenza B virus
  source 1..60: mol_type = other DNA
gaatttaatacgactcactatagggataactcaacagatcggtgctcttgaccaaattgg

SEQ ID NO: 107 - DNA, 36 bp, synthetic construct
  misc_feature 1..36: note = Hybridization probe sequence for Influenza B virus detection
  source 1..36: mol_type = other DNA
ttttccaattcgagcagctgaaactgcggtgttttt

SEQ ID NO: 108 - DNA, 21 bp, synthetic construct
  misc_feature 1..21: note = Detector probe sequence for Influenza A and Influenza B virus and human Beta-2-microglobuline gene detection
  modified_base 21: mod_base = OTHER; note = Thymidine at position 21 is biotinylated
  source 1..21: mol_type = other DNA
tttctaccgtactctagcttt

SEQ ID NO: 109 - DNA, 21 bp, synthetic construct
  misc_feature 1..21: note = Detector probe sequence for Influenza A and Influenza B virus and human Beta-2-microglobuline gene detection
  modified_base 21: mod_base = OTHER; note = Thymidine at position 21 is biotinylated
  source 1..21: mol_type = other DNA
tttctgttgagttatcccttt

SEQ ID NO: 110 - DNA, 41 bp, synthetic construct
  misc_feature 1..41: note = Forward primer for human Beta-2-microglobulin gene
  source 1..41: mol_type = other DNA
tttctaccgtactctagctgcctgccgtgtgaaccatgtga

SEQ ID NO: 111 - DNA, 60 bp, synthetic construct
  misc_feature 1..60: note = Reverse primer for human Beta-2-microglobulin gene
  source 1..60: mol_type = other DNA
aatttaatacgactcactatagggataactcaacagtggaattcatccaatccaaatgcg

SEQ ID NO: 112 - DNA, 30 bp, synthetic construct
  misc_feature 1..30: note = Hybridization probe sequence for human Beta-2-microglobulin RNA detection
  source 1..30: mol_type = other DNA
tttttagcatcatggaggtttgaagttttt

SEQ ID NO: 113 - DNA, 20 bp, synthetic construct
  misc_feature 1..20: note = Primer sequence for Listeria species ribosomal RNA analysis
  source 1..20: mol_type = other DNA
cacactggractgagacacg

SEQ ID NO: 114 - DNA, 21 bp, synthetic construct
  misc_feature 1..21: note = Primer sequence for Listeria species ribosomal RNA analysis
  source 1..21: mol_type = other DNA
gtattaccgcggctgctggca

SEQ ID NO: 115 - DNA, 40 bp, synthetic construct
  misc_feature 1..40: note = Primer sequence for Listeria species ribosomal RNA analysis
  source 1..40: mol_type = other DNA
tttctaccgtactctagctacacactggractgagacacg

SEQ ID NO: 116 - DNA, 58 bp, synthetic construct
  misc_feature 1..58: note = Primer sequence for Listeria species ribosomal RNA analysis
  source 1..58: mol_type = other DNA
gaatttaatacgactcactatagggataactcaacaggtattaccgcggctgctggca

SEQ ID NO: 117 - DNA, 40 bp, synthetic construct
  misc_feature 1..40: note = Forward primer sequence for a hypervariable 3 region of 16S rDNA
  source 1..40: mol_type = other DNA
tttctaccgtactctagctcacattggractgagacacgg

SEQ ID NO: 118 - DNA, 57 bp, synthetic construct
  misc_feature 1..57: note = Reverse primer sequence for a hypervariable 3 region of 16S rDNA
  source 1..57: mol_type = other DNA
aatttaatacgactcactatagggataactcaacaggtattaccgcggctgctggca

SEQ ID NO: 119 - DNA, 40 bp, synthetic construct
  misc_feature 1..40: note = Forward primer sequence for a hypervariable 6 region of 16S rDNA
  source 1..40: mol_type = other DNA
tttctaccgtactctagctcgcacaagcggtggagcatgt

SEQ ID NO: 120 - DNA, 58 bp, synthetic construct
  misc_feature 1..58: note = Reverse primer sequence for a hypervariable 6 region of 16S rDNA
  source 1..58: mol_type = other DNA
aatttaatacgactcactatagggataactcaacagacgagctgacgacagccatgca

SEQ ID NO: 121 - DNA, 40 bp, synthetic construct
  misc_feature 1..40: note = Forward primer sequence for imp-1 RNA
  source 1..40: mol_type = other DNA
tttctaccgtactctagctgaatagrrtggcttaaytctc

SEQ ID NO: 122 - DNA, 54 bp, synthetic construct
  misc_feature 1..54: note = Reverse primer sequence for imp-1 RNA
  source 1..54: mol_type = other DNA
aatttaatacgactcactatagggataactcaacagccaaacyactasgttatc

SEQ ID NO: 123 - DNA, 40 bp, synthetic construct
  misc_feature 1..40: note = Forward primer sequence for kpc RNA
  source 1..40: mol_type = other DNA
tttctaccgtactctagctcggccgccgtgcaatacagtg

SEQ ID NO: 124 - DNA, 57 bp, synthetic construct
  misc_feature 1..57: note = Reverse primer sequence for kpc RNA
  source 1..57: mol_type = other DNA
aatttaatacgactcactatagggataactcaacaggccgcccaactccttcagcaa

SEQ ID NO: 125 - DNA, 39 bp, synthetic construct
  misc_feature 1..39: note = Forward primer sequence for oxa-48 RNA
  source 1..39: mol_type = other DNA
tttctaccgtactctagctcttaaacgggcgaaccaagc

SEQ ID NO: 126 - DNA, 56 bp, synthetic construct
  misc_feature 1..56: note = Reverse primer sequence for oxa-48 RNA
  source 1..56: mol_type = other DNA
aatttaatacgactcactatagggataactcaacaggttcatccttaaccacgccc

SEQ ID NO: 127 - DNA, 41 bp, synthetic construct
  misc_feature 1..41: note = Forward primer sequence for ndm-1 RNA
  source 1..41: mol_type = other DNA
tttctaccgtactctagcttgaccagaccgcccagatcctc

SEQ ID NO: 128 - DNA, 57 bp, synthetic construct
  misc_feature 1..57: note = Reverse primer sequence for ndm-1 RNA
  source 1..57: mol_type = other DNA
aatttaatacgactcactatagggataactcaacagccgccagcgcgaccggcaggt

SEQ ID NO: 129 - DNA, 41 bp, synthetic construct
  misc_feature 1..41: note = Forward primer sequence for vim RNA
  source 1..41: mol_type = other DNA
tttctaccgtactctagctaatggtctcattgtccgtgatg

SEQ ID NO: 130 - DNA, 53 bp, synthetic construct
  misc_feature 1..53: note = Reverse primer sequence for vim RNA
  source 1..53: mol_type = other DNA
aatttaatacgactcactatagggataactcaacagtcgcaccccacgctgta

SEQ ID NO: 131 - DNA, 41 bp, synthetic construct
  misc_feature 1..41: note = Forward primer sequence for mcr-1 RNA
  source 1..41: mol_type = other DNA
aatttaccgtactctagcttagctgaacatacacggcacag

SEQ ID NO: 132 - DNA, 58 bp, synthetic construct
  misc_feature 1..58: note = Reverse primer sequence for mcr-1 RNA
  source 1..58: mol_type = other DNA
aatttaatacgactcactatagggataactcaacagccaaagacgcggtacaagcaac

SEQ ID NO: 133 - DNA, 39 bp, synthetic construct
  misc_feature 1..39: note = Forward primer sequence for ctx-m RNA
  source 1..39: mol_type = other DNA
tttctaccgtactctagctaccaacgatatcgcggtgat

SEQ ID NO: 134 - DNA, 54 bp, synthetic construct
  misc_feature 1..54: note = Reverse primer sequence for ctx-m RNA
  source 1..54: mol_type = other DNA
aatttaatacgactcactatagggataactcaacagacatcgcgacggctttct

SEQ ID NO: 135 - DNA, 41 bp, synthetic construct
  misc_feature 1..41: note = Forward primer sequence for mecA RNA
  source 1..41: mol_type = other DNA
tttctaccgtactctagcttaaaccacccaatttgtctgcc

SEQ ID NO: 136 - DNA, 58 bp, synthetic construct
  misc_feature 1..58: note = Reverse primer sequence for mecA RNA
  source 1..58: mol_type = other DNA
aatttaatacgactcactatagggataactcaacagatggtatgcaacaagtcgtaaa

SEQ ID NO: 137 - DNA, 42 bp, synthetic construct
  misc_feature 1..42: note = Forward primer sequence for mecC RNA
  source 1..42: mol_type = other DNA
tttctaccgtactctagctgaaaaaaaggcttagaacgcctc

SEQ ID NO: 138 - DNA, 58 bp, synthetic construct
  misc_feature 1..58: note = Reverse primer sequence for mecC RNA
  source 1..58: mol_type = other DNA
aatttaatacgactcactatagggataactcaacaggaagatcttttccgttttcagc

SEQ ID NO: 139 - DNA, 39 bp, synthetic construct
  misc_feature 1..39: note = Forward primer sequence for vanA RNA
  source 1..39: mol_type = other DNA
tttctaccgtactctagctagccggaaaaaggctctgaa

SEQ ID NO: 140 - DNA, 56 bp, synthetic construct
  misc_feature 1..56: note = Reverse primer sequence for vanA RNA
  source 1..56: mol_type = other DNA
aatttaatacgactcactatagggataactcaacagttttgccgtttcctgtatcc

SEQ ID NO: 141 - DNA, 40 bp, synthetic construct
  misc_feature 1..40: note = Forward primer sequence for for vanB RNA
  source 1..40: mol_type = other DNA
tttctaccgtactctagctgatttgattgtcggcgaagtg

SEQ ID NO: 142 - DNA, 55 bp, synthetic construct
  misc_feature 1..55: note = Reverse primer sequence for vanB RNA
  source 1..55: mol_type = other DNA
aatttaatacgactcactatagggataactcaacagtcctgatggatgcggaaga

SEQ ID NO: 143 - DNA, 41 bp, synthetic construct; identical to SEQ ID NO: 110
  misc_feature 1..41: note = Forward primer sequence for human beta-2 microglobulin mRNA
  source 1..41: mol_type = other DNA

SEQ ID NO: 144 - DNA, 60 bp, synthetic construct; identical to SEQ ID NO: 111
  misc_feature 1..60: note = Reverse primer sequence for human beta-2 microglobulin mRNA
  source 1..60: mol_type = other DNA

SEQ ID NO: 145 - DNA, 16 bp, synthetic construct
  misc_feature 1..16: note = Probe sequence for a hypervariable 3a region in Enterococcus faecium
  source 1..16: mol_type = other DNA
caaggatgagagtaac

SEQ ID NO: 146 - DNA, 18 bp, synthetic construct
  misc_feature 1..18: note = Probe sequence for a hypervariable 3a region in Staphylococcus aureus
  source 1..18: mol_type = other DNA
gaagaacatatgtgtaag

SEQ ID NO: 147 - DNA, 16 bp, synthetic construct
  misc_feature 1..16: note = Probe sequence for a hypervariable 3a region in Klebsiella pneumoniae1
  source 1..16: mol_type = other DNA
aggcgatgaggttaat

SEQ ID NO: 148 - DNA, 16 bp, synthetic construct
  misc_feature 1..16: note = Probe sequence for a hypervariable 3a region in Klebsiella pneumoniae2
  source 1..16: mol_type = other DNA
aggcgataaggttaat

SEQ ID NO: 149 - DNA, 16 bp, synthetic construct
  misc_feature 1..16: note = Probe sequence for a hypervariable 3a region in Klebsiella pneumoniae3, Klebsiella pneumoniae5, Klebsiella pneumoniae7, E. (K.) Aerogenes and for a hypervariable 3c region in Klebsiella pneumoniae5
  source 1..16: mol_type = other DNA
gaaggcgttaaggtta

SEQ ID NO: 150 - DNA, 17 bp, synthetic construct
  misc_feature 1..17: note = Probe sequence for a hypervariable 3a region in Klebsiella pneumoniae4
  source 1..17: mol_type = other DNA
aggcgataaggttaatn

SEQ ID NO: 151 - DNA, 14 bp, synthetic construct
  misc_feature 1..14: note = Probe sequence for a hypervariable 3a region in Klebsiella pneumoniae6
  source 1..14: mol_type = other DNA
aaggcggtgaggtt

SEQ ID NO: 152 - DNA, 17 bp, synthetic construct
  misc_feature 1..17: note = Probe sequence for a hypervariable 3a region in Acinetobacter baumannii
  source 1..17: mol_type = other DNA
gctactttagttaatac

SEQ ID NO: 153 - DNA, 16 bp, synthetic construct
  misc_feature 1..16: note = Probe sequence for a hypervariable 3a region in Pseudomonas aeruginosa
  source 1..16: mol_type = other DNA
aagggcagtaagttaa

SEQ ID NO: 154 - DNA, 15 bp, synthetic construct
  misc_feature 1..15: note = Probe sequence for a hypervariable 3a region in Enterobacter spp cloacae
  source 1..15: mol_type = other DNA
aaggtgttgtggtta

SEQ ID NO: 155 - DNA, 16 bp, synthetic construct
  misc_feature 1..16: note = Probe sequence for a hypervariable 3a region in Enterobacter spp asburiae and spp hormaechei
  source 1..16: mol_type = other DNA
aaggcgataaggttaa

SEQ ID NO: 156 - DNA, 17 bp, synthetic construct
  misc_feature 1..17: note = Probe sequence for a hypervariable 3a region in Escherichia coli
  source 1..17: mol_type = other DNA
aagggagtaaagttaat

SEQ ID NO: 157 - DNA, 17 bp, synthetic construct
  misc_feature 1..17: note = Probe sequence for a hypervariable 3c region in Enterococcus faecium
  source 1..17: mol_type = other DNA
taactgttcatcccttg

SEQ ID NO: 158 - DNA, 15 bp, synthetic construct
  misc_feature 1..15: note = Probe sequence for a hypervariable 3c region in Staphylococcus aureus
  source 1..15: mol_type = other DNA
tgtgcacatcttgac

SEQ ID NO: 159 - DNA, 18 bp, synthetic construct
  misc_feature 1..18: note = Probe sequence for a hypervariable 3c region in Klebsiella pneumoniae1, Klebsiella pneumoniae6 and Klebsiella pneumoniae7
  source 1..18: mol_type = other DNA
ataacctcatcgattgac

SEQ ID NO: 160 - DNA, 16 bp, synthetic construct
  misc_feature 1..16: note = Probe sequence for a hypervariable 3c region in Klebsiella pneumoniae2, Klebsiella pneumoniae4, Enterobacter spp asburiae and spp hormaechei
  source 1..16: mol_type = other DNA
taaccttgtcgattga

SEQ ID NO: 161 - DNA, 16 bp, synthetic construct
  misc_feature 1..16: note = Probe sequence for a hypervariable 3c region in Klebsiella pneumoniae3 and E.(K.) aerogenes
  source 1..16: mol_type = other DNA
taaccttggcgattga

SEQ ID NO: 162 - DNA, 16 bp, synthetic construct
  misc_feature 1..16: note = Probe sequence for a hypervariable 3c region in Acinetobacter baumannii
  source 1..16: mol_type = other DNA
atacctagagatagtg

SEQ ID NO: 163 - DNA, 16 bp, synthetic construct
  misc_feature 1..16: note = Probe sequence for a hypervariable 3c region in Pseudomonas aeruginosa
  source 1..16: mol_type = other DNA
ataccttgctgtttga

SEQ ID NO: 164 - DNA, 16 bp, synthetic construct
  misc_feature 1..16: note = Probe sequence for a hypervariable 3c region in Enterobacter spp cloacae
  source 1..16: mol_type = other DNA
cgcagcaattgacgtt

SEQ ID NO: 165 - DNA, 16 bp, synthetic construct
  misc_feature 1..16: note = Probe sequence for a hypervariable 3c region in Escherichia coli
  source 1..16: mol_type = other DNA
tacctttgctcattga

SEQ ID NO: 166 - DNA, 15 bp, synthetic construct
  misc_feature 1..15: note = Probe sequence for a hypervariable 3e region in Enterococcus faecium
  source 1..15: mol_type = other DNA
cggtatctaaccaga

SEQ ID NO: 167 - DNA, 15 bp, synthetic construct
  misc_feature 1..15: note = Probe sequence for a hypervariable 3e region in Staphylococcus aureus
  source 1..15: mol_type = other DNA
acggtacctaatcag

SEQ ID NO: 168 - DNA, 14 bp, synthetic construct
  misc_feature 1..14: note = Probe sequence for a hypervariable 3e region in Klebsiella pneumoniae1, 2, 3, 4, 5, 6, and 7 and Enterobacter spp cloacae, asburiae and hormaechei
  source 1..14: mol_type = other DNA
acgttacccgcaga

SEQ ID NO: 169 - DNA, 14 bp, synthetic construct
  misc_feature 1..14: note = Probe sequence for a hypervariable 3e region in E.(K.) aerogenes
  source 1..14: mol_type = other DNA
acgttactcgcaga

SEQ ID NO: 170 - DNA, 16 bp, synthetic construct
  misc_feature 1..16: note = Probe sequence for a hypervariable 3e region in Pseudomonas aeruginosa
  source 1..16: mol_type = other DNA
cgttaccaacagaata

SEQ ID NO: 171 - DNA, 16 bp, synthetic construct
  misc_feature 1..16: note = Probe sequence for a hypervariable 6a region of Enterococcus faecium
  source 1..16: mol_type = other DNA
ccttaccaggtcttga

SEQ ID NO: 172 - DNA, 16 bp, synthetic construct
  misc_feature 1..16: note = Probe sequence for a hypervariable 6a region of Staphylococcus aureus
  source 1..16: mol_type = other DNA
cttaccaaatcttgac

SEQ ID NO: 173 - DNA, 15 bp, synthetic construct
  misc_feature 1..15: note = Probe sequence for a hypervariable 6a region in Klebsiella pneumoniae1, 2, 3, 4, 5, 6, and 7 and Escherichia coli
  source 1..15: mol_type = other DNA
tacctggtcttgaca

SEQ ID NO: 174 - DNA, 14 bp, synthetic construct
  misc_feature 1..14: note = Probe sequence for a hypervariable 6a region of Acinetobacter baumannii
  source 1..14: mol_type = other DNA
acctggccttgaca

SEQ ID NO: 175 - DNA, 14 bp, synthetic construct
  misc_feature 1..14: note = Probe sequence for a hypervariable 6a region of Enterobacter spp cloacae
  source 1..14: mol_type = other DNA
tacctggtcttgac

SEQ ID NO: 176 - DNA, 15 bp, synthetic construct
  misc_feature 1..15: note = Probe sequence for a hypervariable 6a region of Enterobacter spp asburiae and spp hormaechei and E.(K.) aerogenes
  source 1..15: mol_type = other DNA
ccttacctactcttg

SEQ ID NO: 177 - DNA, 15 bp, synthetic construct
  misc_feature 1..15: note = Probe sequence for a hypervariable 6c region of Enterococcus faecium
  source 1..15: mol_type = other DNA
agagcttccccttcg

SEQ ID NO: 178 - DNA, 17 bp, synthetic construct
  misc_feature 1..17: note = Probe sequence for a hypervariable 6c region of Staphyloccus aureus
  source 1..17: mol_type = other DNA
atcctttgacaactcta

SEQ ID NO: 179 - DNA, 14 bp, synthetic construct
  misc_feature 1..14: note = Probe sequence for a hypervariable 6c region of Klebsiella pneumoniae1, 3, 4, 6, 7 and Enterobacter spp cloacae
  source 1..14: mol_type = other DNA
ccacagaactttcc

SEQ ID NO: 180 - DNA, 15 bp, synthetic construct
  misc_feature 1..15: note = Probe sequence for a hypervariable 6c region of Klebsiella pneumoniae2 and Klebsiella pneumoniae5
  source 1..15: mol_type = other DNA
tccacagaacttagc

SEQ ID NO: 181 - DNA, 17 bp, synthetic construct
  misc_feature 1..17: note = Probe sequence for a hypervariable 6c region of Acinetobacter baumannii
  source 1..17: mol_type = other DNA
atactagaaactttcca

SEQ ID NO: 182 - DNA, 15 bp, synthetic construct
  misc_feature 1..15: note = Probe sequence for a hypervariable 6c region of Pseudomonas aeruginosa
  source 1..15: mol_type = other DNA
tgctgagaactttcc

SEQ ID NO: 183 - DNA, 15 bp, synthetic construct
  misc_feature 1..15: note = Probe sequence for a hypervariable 6c region of Enterobacter spp asburiae
  source 1..15: mol_type = other DNA
tccagagaactttcc

SEQ ID NO: 184 - DNA, 14 bp, synthetic construct
  misc_feature 1..14: note = Probe sequence for a hypervariable 6c region of Enterobacter spp Hormaechei and E.(K.) aerogenes
  source 1..14: mol_type = other DNA
ccagagaacttagc

SEQ ID NO: 185 - DNA, 15 bp, synthetic construct
  misc_feature 1..15: note = Probe sequence for a hypervariable 6c region of Escherichia coli
  source 1..15: mol_type = other DNA
ccacggaagttttca

SEQ ID NO: 186 - DNA, 14 bp, synthetic construct
  misc_feature 1..14: note = Probe sequence for a hypervariable 6g region of Enterococcus faecium
  source 1..14: mol_type = other DNA
gggcaaagtgacag

SEQ ID NO: 187 - DNA, 14 bp, synthetic construct
  misc_feature 1..14: note = Probe sequence for a hypervariable 6g region of Staphylococcus aureus
  source 1..14: mol_type = other DNA
ggacaaagtgacag

SEQ ID NO: 188 - DNA, 14 bp, synthetic construct
  misc_feature 1..14: note = Probe sequence for a hypervariable 6g region of Klebsiella pneumoniae1, 2, 3, 4, 5, 6, and 7 and Enterobacter spp cloacae
  source 1..14: mol_type = other DNA
ggaactgtgagaca

SEQ ID NO: 189 - DNA, 16 bp, synthetic construct
  misc_feature 1..16: note = Probe sequence for a hypervariable 6g region of Acinetobacter baumannii
  source 1..16: mol_type = other DNA
gaatctagatacaggt

SEQ ID NO: 190 - DNA, 14 bp, synthetic construct
  misc_feature 1..14: note = Probe sequence for a hypervariable 6g region of Pseudomonas aeruginosa
  source 1..14: mol_type = other DNA
gaacagagacacag

SEQ ID NO: 191 - DNA, 14 bp, synthetic construct
  misc_feature 1..14: note = Probe sequence for a hypervariable 6g region of Enterobacter spp asburiae, Enterobacter spp hormaechei and E.(K.) aerogenes
  source 1..14: mol_type = other DNA
ggaactctgagaca

SEQ ID NO: 192 - DNA, 13 bp, synthetic construct
  misc_feature 1..13: note = Probe sequence for a hypervariable 6g region of Escherichia coli
  source 1..13: mol_type = other DNA
gggaaccgtgaga

SEQ ID NO: 193 - DNA, 16 bp, synthetic construct
  misc_feature 1..16: note = Probe sequence for negative control
  source 1..16: mol_type = other DNA
tacctatgctgattca

SEQ ID NO: 194 - DNA, 15 bp, synthetic construct
  misc_feature 1..15: note = Probe sequence for Pan-hypervariable 3 control
  source 1..15: mol_type = other DNA
cctacgggaggcagc

SEQ ID NO: 195 - DNA, 22 bp, synthetic construct
  misc_feature 1..22: note = Probe sequence for imp-1 antibiotic resistance target
  source 1..22: mol_type = other DNA
ctaccctggcccaggtcacact

SEQ ID NO: 196 - DNA, 20 bp, synthetic construct
  misc_feature 1..20: note = Probe sequence for kpc antibiotic resistance target
  source 1..20: mol_type = other DNA
taacgccgccgccaatttgt

SEQ ID NO: 197 - DNA, 22 bp, synthetic construct
  misc_feature 1..22: note = Probe sequence for oxa-48 antibiotic resistance target
  source 1..22: mol_type = other DNA
ccaatagcttgatcgccctcga

SEQ ID NO: 198 - DNA, 20 bp, synthetic construct
  misc_feature 1..20: note = Probe sequence for ndm-1 antibiotic resistance target
  source 1..20: mol_type = other DNA
actggatcaagcaggagatc

SEQ ID NO: 199 - DNA, 20 bp, synthetic construct
  misc_feature 1..20: note = Probe sequence for vim antibiotic resistance target
  source 1..20: mol_type = other DNA
tgatgagttgcttttgattg

SEQ ID NO: 200 - DNA, 22 bp, synthetic construct
  misc_feature 1..22: note = Probe sequence for mcr-1 antibiotic resistance target
  source 1..22: mol_type = other DNA
aggcgtggcttacgcatatcag

SEQ ID NO: 201 - DNA, 20 bp, synthetic construct
  misc_feature 1..20: note = Probe sequence for ctx-m antibiotic resistance target
  source 1..20: mol_type = other DNA
agatcgtgcgccgctgattc

SEQ ID NO: 202 - DNA, 22 bp, synthetic construct
  misc_feature 1..22: note = Probe sequence for mecA antibiotic resistance target
  source 1..22: mol_type = other DNA
cattttgagttctgcagtaccg

SEQ ID NO: 203 - DNA, 22 bp, synthetic construct
  misc_feature 1..22: note = Probe sequence for mecC antibiotic resistance target
  source 1..22: mol_type = other DNA
aaacctttagacacattattgg

SEQ ID NO: 204 - DNA, 22 bp, synthetic construct
  misc_feature 1..22: note = Probe sequence for vanA antibiotic resistance target
  source 1..22: mol_type = other DNA
cagttataaccgttcccgcaga

SEQ ID NO: 205 - DNA, 22 bp, synthetic construct
  misc_feature 1..22: note = Probe sequence for vanB antibiotic resistance target
  source 1..22: mol_type = other DNA
tcaaatccggctgagccacggt

SEQ ID NO: 206 - DNA, 20 bp, synthetic construct
  misc_feature 1..20: note = Probe sequence for human B2M antibiotic resistance target
  source 1..20: mol_type = other DNA
agcatcatggaggtttgaag

(19) United States
(12) Patent Application Publication
(10) Pub. No.: US 2024/0384359 A1
(43) Pub. Date: Nov. 21, 2024

(54) Transcription Mediated Amplification Methods for RNA Detection

(71) Applicant: PathogenDx, Inc., Scottsdale, AZ (US)

(72) Inventors: Michael E. Hogan, Stony Brook, NY (US); Fushi Wen, Tucson, AZ (US); Frederick H. Eggers, Sahuarita, AZ (US)

(73) Assignee: PathogenDx, Inc., Scottsdale, AZ (US)

(21) Appl. No.: 18/794,012

(22) Filed: Aug. 5, 2024

Related U.S. Application Data
(63) Continuation-in-part of application No. 17/840,835, filed on Jun. 15, 2022, now Pat. No. 12,054,794.
(60) Provisional application No. 63/210,934, filed on Jun. 15, 2021.

(51) Int. Cl. C12Q 1/689; C12Q 1/6865

(52) U.S. Cl. CPC C12Q 1/689; C12Q 1/6865; C12Q 2600/106; C12Q 2600/156

(57) ABSTRACT

Provided herein are methods for identifying antibiotic resistance in a subject in need thereof and for detecting drug resistance in a blood-borne pathogenic bacteria. The methods amplify RNA isolated from whole blood in a multiplexed, isothermal nucleic acid sequence-based amplification (NASBA) reaction using at least one isothermal primer pair with nucleotide sequences specific to a drug resistance marker and with a labeled detector probe binding site and a T7 promoter sequence. Amplicons are hybridized to drug resistance marker probes and to a labeled universal detector probe. A signal indicates drug resistance in the blood-borne pathogenic bacteria.

### CROSS-REFERENCE TO RELATED APPLICATIONS

This continuation-in-part application claims benefit of priority under 35 U.S.C § 120 of pending non-provisional application U.S. Ser. No. 17/840,835, filed Jun. 15, 2022,which claims benefit of priority under 35 U.S.C. § 119 (e) of provisional application U.S. Ser. No. 63/210,934, filed Jun. 15, 2021, the entireties of which are hereby incorporated by reference.

### SEQUENCE LISTING

A sequence listing is electronically submitted in XML format in compliance with 37 C.F.R. § 1.831 (a) and is incorporated by reference herein. The XML file is named D7775CIPSEQ, was created on Aug. 4, 2024 and is 267 KB in size.

### BACKGROUND OF THE INVENTION

### Field of the Invention

The present invention relates to the field of multiplex-based pathogen detection and analysis. More particularly, the present invention relates to methods combining multiplexed, isothermal nucleic acid sequence-based amplification (NASBA) reactions with microarray analysis for detecting the presence of an RNA associated with antibiotic resistance in pathogenic bacteria.

### Description of the Related Art

In 2019, the U.S. Centers for Disease Control and Prevention (CDC) declared that “Antibiotic resistance is one of the greatest global public health challenges of our time” (1). The most urgent and serious threats are associated with nosocomial infections1, and what are commonly known as the ESKAPE+ pathogens (2). The widespread and/or indiscriminate use of broad-spectrum antibiotics contributes to antibiotic resistance, a problem that is exacerbated by continued reliance on culture-based diagnostics, and/or cumbersome molecular diagnostics. The challenge for molecular diagnostics stems from the fact that direct-from-sample antimicrobial drug susceptibility testing (DST) is inherently a trace detection problem that requires large starting sample volumes, a seamless interface between amplification and detection functions, and multiplexing capabilities to simultaneously identify pathogens to the species level and detect their drug resistance determinants.

PCR technology has dominated diagnostics and public health screening during pandemics. Independent of the test developer, PCR has been shown to have an unusually high false negative rate (15% up to 30%). Meta-analysis has shown that the false negative rate for PCR is high when pathogen load is low. This renders traditional PCR ineffective as a tool for early detection of weak symptomatic carriers while also lessening its value in epidemiology. Moreover, traditional PCR is unable to meet the increasing demand for rapid point-of-care and field testing, which are crucial for management of diseases during pandemics.

It is well known in the art that DNA and RNA analytes may be enzymatically amplified prior to analysis by hybridization to cognate nucleic acid probes. Such amplification methods include thermal cycling (PCR) and a number of reactions which are not based on thermal cycling and thus as a class are referred to as “Isothermal”. These include loop-assisted isothermal amplification (LAMP) and Recombinase Polymerase Amplification (RPA), which generate amplified DNA fragments, and isothermal amplification methods including Transcription Mediated Amplification (TMA) especially the NASBA variant of TMA, “TMA (NASBA)” which generate amplified RNA fragments. A known limitation of these isothermal methods is the difficulty to introduce dyes or other markers and tags into the amplified nucleic acid fragments. This is particularly difficult with conventional TMA methods, where the amplification reaction produces single stranded RNA amplicons.

Thus, conventional isothermal amplification and detection methods are unsuitable for rapid point-of-care drug susceptibility testing. Particularly, the art is deficient in sample-to-answer multiplex isothermal NASBA amplification and microarray analysis method to detect antibiotic resistance in pathogenic bacteria. The present invention fulfills this long-standing need and desire in the art.

### SUMMARY OF THE INVENTION

The present invention is directed to a method for identifying antibiotic resistance in a subject in need thereof. In the method a whole blood sample is obtained from the subject and RNA is extracted from the whole blood sample. A multiplexed, isothermal nucleic acid sequence-based amplification (NASBA) reaction is performed on the RNA to generate a plurality of RNA amplicons using at least one forward isothermal primer and at least one reverse isothermal primer where each comprises nucleotide sequences specific to an antibiotic resistance determinant in a blood-borne pathogenic bacteria and specific to a detector probe binding site and where each reverse isothermal primer comprises a T7 promoter sequence. The plurality of RNA amplicons is hybridized at room temperature to a plurality of labeled universal detector probes and to a plurality of hybridization probes each comprising a nucleotide sequence complementary to the nucleotide sequence specific to the antibiotic resistance determinant in the blood-borne pathogenic bacteria; where the hybridization probes are attached to a solid substrate on a microarray via an oligonucleotide linker to form a three dimensional structure thereon. The microarray is washed at room temperature at least once and the microarray is imaged to detect at least one signal from at least one of the plurality of labeled universal detector probes, thereby identifying antibiotic resistance in the subject.

The present invention also is directed to a method for detecting drug resistance in a blood-borne pathogenic bacteria. In the method a whole blood sample is obtained from a subject with an infection caused by the blood-borne pathogenic bacteria and RNA is extracted from the whole blood sample. A multiplexed, isothermal nucleic acid sequence-based amplification (NASBA) reaction is performed on the RNA to generate a plurality of RNA amplicons using at least one isothermal primer pair, where the isothermal primer pairs comprise a forward isothermal primer and a reverse isothermal primer each of which comprising nucleotide sequences specific to a drug resistance marker in the blood borne pathogenic bacteria and to a detector probe binding site, where the reverse isothermal primer further comprises a T7 promoter sequence. The plurality of RNA amplicons are hybridized at room temperature to a plurality of fluorescent labeled universal detector probes and to a plurality of hybridization probes each comprising a nucleotide sequence complementary to the nucleotide sequence specific to the antibiotic resistance genetic marker; where the hybridization probes are attached to a solid substrate on a microarray via an oligonucleotide linker to form a three dimensional structure thereon. The microarray is washed at room temperature at least once and the microarray is imaged to detect at least one signal from at least one of the plurality of labeled universal detector probes, thereby detecting drug resistance in the blood borne pathogenic bacteria.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

These and other features, aspects, and advantages of the embodiments of the present disclosure will become better understood when the following detailed description is read with reference to the accompanying drawings in which like characters represent like parts throughout the drawing, wherein:

FIG. 1 illustrates the steps in nucleic acid sequence-based Transcription Mediated Amplification (TMA) of target RNA.

FIG. 2 illustrates hybridization of the amplified RNA target to hybridization probes on the microarray substrate.

FIGS. 3A-3D show the results in identifying variants in clinical samples.

FIG. 4 is a comparison of VOC/I with GIV WT.

FIG. 5 is a comparison of VOC/I Cv Ct for correct variant identification vs Cv Ct to detect Cov-2 spike.

FIG. 6 is a comparison of VOC/I Rv array Ct vs the Cv Ct array ability to detect CoV-2 spike.

FIG. 7 is a comparison of VOC/I Rv array Ct vs the Cv array ability to correctly identify a variant.

FIG. 8 shows the relative fluorescent unit (RFU) of hybridization signals derived via the method provided herein of SARS-Cov-2 (with multiple probes), influenza A, influenza B, and B2M. Templates are genomic RNA of each virus and Human Reference RNA for B2M. 10,000 genome copies/reaction of templates are used. SARS-Cov-2 genomic RNA is from wild type (Wuhan strain) of CoV-2, influenza A gRNA is from H1N1 influenza A subtype, and influenza B gRNA is from Yamagata lineage of influenza B.

FIGS. 9A-9E show the relative fluorescent unit (RFU) of hybridization signals derived via the method provided herein from SARS-Cov-2 clinical positive and negative samples, including 2 Omicron BA1 (BA1-1, BA1-2), 4 Omicron BA2 (BA2-1, BA2-2, BA2-3, BA2-4), 2 Delta (Delta-1, Delta-2), 2 clinical negative (Clinical Neg-1, Clinical Neg-2), 1 Human-reference-RNA (hmRfRNA), and a No-Template-Control (NTC).

FIGS. 10A-10C show the relative fluorescent unit (RFU) of hybridization signals derived via the method provided herein from SARS-Cov-2 genomic RNA at concentration of 10,000, 1000, and 100 genome copies/reaction.

FIG. 11 shows the relative fluorescent unit (RFU) of hybridization signals derived via the method provided herein from SARS-Cov-2 genomic RNA at concentration of 10,000, 1000, and 100 genome copies/reaction.

FIGS. 12A-12B show the relative fluorescent unit (RFU) of hybridization signals derived via the method provided herein from 2 of SARS-Cov-2 Omicron BA-1 and 2 BA-2 clinical samples detected by PDx-NASBA and PDx-Detect-CV+ assays.

FIGS. 13A-13B show the relative fluorescent unit (RFU) of hybridization signals derived via the method provided herein from detection of SARS-Cov-2 wild type (Wuhan) genomic RNA at 40,000 genome copies/reaction with single plex primer set. In the SAPE detection, biotinylated detector probe binds with SAPE (Streptavidin, R-Phycoerythrin conjugate) (FIG. 13A) to show fluorescent signals, while in the Cy3 detection (FIG. 13B), Cy3 labeled detector probe shows fluorescent signals.

FIG. 14A-14C illustrates the isothermal, multiplexed amplification, and microarray detection scheme. In FIG. 14A each gene-specific primer (P1 or P2) contains a universal detection probe sequence (red bar). Antisense RNA from the linear phase feeds into the cyclic phase (FIG. 14B). Antisense RNA transcripts from the cyclic phase contain a T7 promoter sequence (orange bar), thus accelerating the reaction. Each dsDNA product in either the linear or cyclic phases of the reaction can produce ˜100 RNA(−) transcripts. In FIG. 14C amplified RNA transcripts (RNA(−)) are detected by room temperature hybridization in an all-in-one sandwich assay that contains labeled, universal detector probes. Green=RNA; blue=gene-specific sequences on P1 or P2; grey=newly synthesized DNA; purple=universal detector probe. All microarray probes are designed to hybridize with RNA(−) transcripts.

FIGS. 15A-15B demonstrate multiplexed (n=4) isothermal NASBA amplification and microarray detection of microbial rRNA and mRNA in <4 hrs. The test targets 16S and 23S rRNA in S. enterica (FIG. 15A), and the Listeria monocytogenes specific gene hly (FIG. 15B). RFU=relative fluorescent units; NTC=no template control.

FIGS. 16A-16H show RNA recovery and multiplexed NASBA/microarray detection of ESKAPE+ pathogens from whole blood. Signals are the average from 3 replicate spots per probe. Blue bars=PBS samples; orange bars=spiked whole blood.

FIG. 17 shows that doubling the detector probe concentration at least doubles signal intensity. The experiment from FIGS. 16A-16H was repeated, except that twice the concentration of detector probe was used. RFU=relative fluorescence units and values are the sum RFU of all species-specific probes (at least 6 unique probes per species). Blue bars=detector probe concentration from FIGS. 16A-16H and orange bars are 2× the detector probe concentration.

FIG. 18 illustrates a plate layout for the multiplex isothermal NASBA amplification and hybridization assay performed on whole blood. Grey=TruTip sample preparation steps; orange=isothermal amplification steps; yellow=microarray steps. Eight parallel microarrays are printed on a single 1×3″ substrate and surrounded by a black frame seal.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method described herein can be implemented with respect to any other method described herein.

As used herein, the term “or” in the claims is used to mean “and/or” unless explicitly indicated to refer to alternatives only or the alternatives are mutually exclusive, although the disclosure supports a definition that refers to only alternatives and “and/or.”

As used herein, “comprise” and its variations, such as “comprises” and “comprising,” will be understood to imply the inclusion of a stated item, element or step or group of items, elements, or steps but not the exclusion of any other item, element or step or group of items, elements, or steps unless the context requires otherwise. Similarly, “another” or “other” may mean at least a second or more of the same or different claim element or components thereof.

As used herein, “consist of” and its variations, such as “consists of” and “consisting of,” will be understood to imply the inclusion of a stated item, element or step or group of items, elements or steps but not the inclusion of any other item, element or step or group of items, elements or steps.

As used herein, the term “about” refers to a numeric value, including, for example, whole numbers, fractions, and percentages, whether or not explicitly indicated. The term “about” generally refers to a range of numerical values (e.g., ±5-10% of the recited value) that one of ordinary skill in the art would consider equivalent to the recited value (e.g., having the same function or result). In some instances, the term “about” may include numerical values that are rounded to the nearest significant figure.

As used herein, the term “detector probe binding sequence” is a nucleotide sequence located at the 5′ ends or the 3′ ends of the RNA amplicons generated. The detector probe binding sequence comprises a nucleotide sequence that is complementary to a nucleotide sequence in the fluorescent labeled detector probe, which is employed to detect RNA amplicon(s) binding to the hybridization probe(s). As used herein, the term “detector probe binding complementary nucleotide sequence” refers to a sequence in the forward and or reverse primer used in amplification to generate the RNA amplicons comprising at their 3′ ends, the detector probe binding sequence. Thus, the detector probe binding complementary nucleotide sequence is substantially similar to the nucleotide sequence in the fluorescent labeled detector probe.

As used herein, the term “ESKAPE+pathogens” refers to Enterococcus faecium Staphylococcus aureus, Klebsiella pneumoniae, Acinetobacter baumannii, Pseudomonas aeruginosa, Enterobacter species, and Escherichia coli.

As used herein, the terms “antibiotic resistance determinant” and “drug resistance markers” are interchangeable.

In one embodiment of the present invention, there is provided a method for detecting a COVID-19 virus in a sample comprising obtaining the sample; isolating crude nucleic acids therefrom; performing, on the crude nucleic acids, a combined isothermal reverse transcription, RNAse H and isothermal RNA amplification reaction using a plurality of forward primers each comprising at its 5′ end an RNA polymerase promoter sequence or the RNA polymerase promoter sequence and a detector probe nucleotide sequence and a plurality of reverse primers each comprising at its 5′ end a fluorescent labeled detector probe nucleotide sequence to generate a plurality of single stranded RNA amplicons each comprising a sequence complementary to the fluorescent labeled detector probe nucleotide sequence at the 5′ end, the 3′ end or a combination thereof of each of the plurality of RNA amplicons; hybridizing, at an ambient temperature, the plurality of single-stranded RNA amplicons to a plurality of the fluorescent labeled detector probes and a plurality of hybridization probes each comprising a nucleotide sequence complementary to a sequence determinant in the COVID-19 virus, said hybridization probes attached to a solid microarray support; washing the microarray support at least once; and imaging the microarray support to detect at least one fluorescent signal from at least one of the plurality of fluorescent labeled detector probes, thereby detecting the COVID-19 virus in the sample.

Further to this embodiment, the method comprises isolating total RNA or mRNA after the lysing stem where the performing step comprises performing the combined isothermal reverse transcription, RNAse H and isothermal RNA amplification reaction on the total RNA. In another further embodiment the method comprises calculating an intensity of the fluorescent signal to correlate with a copy number of the COVID-19 virus in the sample.

In all embodiments, the COVID-19 virus in the sample is a wild type COVID-19 virus or a clade variant thereof. In a non-limiting example, the clade variant is, but not limited to, a B.1.2, B.1.1.7, B.1.351, B.1.375, B.1.427, B.1.429, B.1.525, B.1.526, P1, P2 and Wuhan. The sample may also comprise a combination of wild type COVID-19 and its clade variants in various proportions.

In all embodiments a combined isothermal reverse transcription and isothermal amplification reaction is performed on the crude nucleic acids to generate a plurality of single stranded RNA amplicons. The combined reaction is performed as a single homogenous reaction in a solution comprising a plurality of forward primers each comprising an RNA polymerase promoter sequence at its 5′ ends and, optionally, a detector probe nucleotide sequence at its 5′ end, a plurality of reverse primers each comprising at its 5′ end, a fluorescent labeled detector probe nucleotide sequence, a reverse transcriptase, an RNase H activity, and an RNA polymerase. In this embodiment, the reverse transcriptase uses the plurality of RNA as template to generate an RNA: cDNA duplex. The RNA in the RNA: cDNA duplex is digested by the intrinsic RNase H activity of the reverse transcriptase (TMA) or an added extrinsic RNAse H activity, TMA (NASBA), to obtain a single stranded cDNA. The cDNA thus obtained is amplified (during isothermal amplification) by the intrinsic DNA polymerase activity or the reverse transcriptase to obtained cDNA duplexes, which are then transcribed by the RNA polymerase to generate a plurality of RNA amplicons corresponding to the plurality of RNA in the sample (hence, Transcription Mediated Amplification). Moreover, each of the plurality of RNA amplicons comprises a sequence complementary to the fluorescent labeled detector probe sequence.

In these embodiments, the binding of the primers suitable to drive reverse transcription is performed at a temperature of about 65° C. or greater, while the reverse transcription, RNase activity and isothermal amplification is performed at a temperature of about 40° C. Typically, the ambient temperature is below a temperature for isothermal reverse transcription and a temperature for isothermal amplification. Particularly, the hybridization step is performed at an ambient temperature between 15° C.-30° C.

In this embodiment, any RNA polymerase promoter sequence is employed. For example, in one aspect, the RNA polymerase promoter sequence is a T7 promoter sequence that would enable generation of single stranded RNA amplicons using a T7RNA polymerase. Exemplary COVID-19 virus specific primers comprising a T7 promoter sequence are shown in Tables 2 and 5 (even SEQ ID NOS.). Exemplary COVID-19 virus specific reverse primers comprising a nucleotide sequence of the fluorescent labeled detector probe are shown in Tables 2 and 5 (odd SEQ ID NOS.). Particularly, the plurality of forward primers comprises the nucleotide sequences of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, or SEQ ID NO: 72 and the plurality of reverse primers comprises the nucleotide sequences of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16 or SEQ ID NO: 73.

The plurality of RNA amplicons generated are hybridized on a microarray support containing a solution comprising a plurality of the fluorescent labeled detector probes and, a plurality of hybridization probes immobilized on the microarray surface. In this embodiment, each of the hybridization probes are attached at specific positions on the microarray support and have a nucleotide sequence complementary to a sequence determinant in the COVID-19 virus. Exemplary COVID-19 virus specific hybridization probes are shown in Table 3 and Table 5. Particularly, the plurality of hybridization probes comprises the nucleotide sequences of SEQ ID NOS: 17-67 or SEQ ID NOS: 74-97.

In these embodiments, the solution phase fluorescent labeled detector probes have a nucleotide sequence that is complementary to a detector probe binding sequence at the 5′ ends and/or the 3′ ends of each of the plurality of RNA amplicons. This enables binding of the fluorescent labeled detector probe to RNA amplicons that are hybridized to the hybridization probe, which are immobilized on the microarray support.

Thus, the fluorescent labeled detector probe enables detection of hybridized RNA amplicon(s) using a fluorescent imager. The fluorescent labeled detector probe has a base length of about 15 nucleotides to about 30 nucleotides and/or a nucleotide sequence with a GC content of about 40% to about 60%. Also in these embodiments the plurality of fluorescent labeled detector probes is in a molar ratio of about 0.1 to about 5 with the plurality of single-stranded RNA amplicons. Particularly, the fluorescent labeled detector probe nucleotide sequence comprises SEQ ID NO: 68, SEQ ID NO: 98, SEQ ID NO: 99, SEQ ID NO: 100, or SEQ ID NO: 101. Also in this embodiment, any fluorescent label may be used in the detector probe including, but not limited to, a CY3, a CY5, SYBR Green, a DYLIGHT™ DY647, an ALEXA FLUOR 647, a DYLIGHT™ DY547, an ALEXA FLUOR 550 phycoerythrin.

After hybridization, the microarray is washed at least once to remove unhybridized amplicons and unbound detector probes. Washed microarrays are imaged to detect at least one from at least one of the plurality of fluorescent labeled detector probed bound to the RNA amplicon(s), thereby detecting presence of the COVID-19 virus in the sample.

In all embodiments and aspects thereof, the sample may comprise at least one of a nasopharyngeal swab, a nasal swab, a mouth swab, a mouthwash, a biopsy sample, a blood sample, an aerosol, or a swab from a hard surface. In one aspect the sample may be any sample obtained from a subject including, but not limited to, a nasopharyngeal swab, a nasal swab, a mouth swab, and a mouthwash (sample obtained by rinsing the subject's buccal cavity). A pooled sample obtained by combining two or more of these samples or by combining samples from multiple subjects also may be used. In another aspect, the sample is an environmental sample obtain from inanimate sources including but is not limited to an aerosol and a hard surface. The aerosol samples may be obtained using commercial air samplers such as for example a Coriolis Micro Air Sampler. A sample from a hard surface may be obtained using a swab. In both aspects, the viruses from samples obtained on swabs are dispersed in a liquid such as phosphate buffered saline. Aerosol samples are transferred into a volume of a liquid such as phosphate buffered saline.

In all of the embodiments and aspects thereof, the reverse primer sequence does not comprise the promoter sequence. Also, in all of the embodiments, the isothermal reaction does not employ any nucleotide bases other than A, G, C, T and U. Further, in all embodiments, the nucleotide sequence of the detector probe has a base length from about 15 nucleotides to about 30 nucleotides. Further still in this embodiment, the nucleotide sequence in the detector probe has a GC content from about 40% to about 60%. In these embodiments the reverse transcription, isothermal amplification, hybridization and imaging steps are performed on separate workstations. Alternatively, the reverse transcription, isothermal amplification, hybridization and imaging steps are performed as sub-assemblies in an integrated device, which is advantageous since it enables full automation with minimal human intervention.

In another embodiment of the present invention, there is provided a method for detecting an RNA of interest in a sample comprising obtaining the sample; isolating nucleic acids from the sample; performing on the nucleic acids, a combined isothermal reverse transcription and isothermal RNA amplification reaction using at least one forward primer comprising at its 5′ end an RNA polymerase promoter sequence or the RNA polymerase promoter sequence and a detector probe nucleotide sequence and at least one reverse primer comprising at its 5′ end a fluorescent labeled detector probe to generate single stranded RNA amplicons each comprising a sequence complementary to the fluorescent labeled detector probe nucleotide sequence; hybridizing the single-stranded RNA amplicons to at least one of the fluorescent labeled detector probes and at least one hybridization probe comprising a nucleotide sequence complementary to a sequence determinant in the RNA of interest, said at least one hybridization probe attached to a solid microarray; washing the microarray at least once; and imaging the microarray to detect at least one fluorescent signal from at least one of the plurality of fluorescent labeled detector probes, thereby detecting the RNA of interest in the sample. Further to this embodiment the method comprises calculating an intensity of the fluorescent signal to correlate with a copy number of the RNA of interest in the sample.

Generally, the overall composition of the primer and probe sequences, the sequence determinants and complementary sequences and fluorescent label on the detector probe are as described supra. In both embodiments, the fluorescent labeled detector probe is in a molar ratio of about 0.1 to about 5 with the single-stranded RNA amplicons. Also in both embodiments the nucleic acids are crude nucleic acids, total RNA, mRNA, or ribosomal RNA. In addition in both embodiments the RNA of interest is a viral RNA, a bacterial RNA, or a pathogenic viral RNA, a fungal RNA or a combination thereof, or a plant RNA, an animal RNA, or a human RNA. For example, the pathogenic viral RNA is isolated from Severe Acute Respiratory Syndrome Coronavirus 2 (COVID-19 virus), a Respiratory Syncytial Virus, a Middle East Respiratory Syndrome coronavirus (MERS-COV), a Severe Acute Respiratory Syndrome Coronavirus (SARS-COV), a 229E Coronavirus, a OC43 Coronavirus, a NL63 Coronavirus, a HKU1 Coronavirus, an Influenza A virus, or an Influenza B virus or a combination thereof. The sample may also comprise a combination of respiratory viruses. The RNA of interest may be from a healthy tissue, or a tissue suspected of being cancerous.

In one aspect of both embodiments, the pathogenic viral RNA is isolated from a wild type COVID-19 virus or a clade variant thereof. In a non-limiting example, the clade variant is, but not limited to B.1.2, B.1.1.7, B.1.351, B.1.375, B.1.427, B.1.429, B.1.525, B.1.526, P1, P2 and Wuhan. The sample also may comprise a combination of wild type COVID-19 and its clade variants in various proportions. In this aspect the nucleotide sequences for the forward primers, the reverse primers, the fluorescent labeled detector probes, and the hybridization probes are as described supra.

In another aspect of both embodiments, the pathogenic viral RNA is isolated from an Influenza A virus. In this aspect the nucleotide sequences for the reverse primers, the fluorescent labeled detector probes, and the hybridization probes are shown in Table 5. Particularly, the forward primer comprises the nucleotide sequence of SEQ ID NO: 102 and the reverse primer comprises the nucleotide sequence of SEQ ID NO: 103. Also, the fluorescent labeled detector probe comprises the nucleotide sequence of SEQ ID NO: 108 or SEQ ID NO: 109 or a combination thereof. In addition the at least one hybridization probe comprises the nucleotide sequences of SEQ ID NO: 104.

In another aspect of both embodiments, the pathogenic viral RNA is isolated from an Influenza B virus. In this aspect the nucleotide sequences for the reverse primers, the fluorescent labeled detector probes, and the hybridization probes are shown in Table 5. Particularly, the forward primer comprises the nucleotide sequence of SEQ ID NO: 105 and the reverse primer comprises the nucleotide sequence of SEQ ID NO: 106. Also, the fluorescent labeled detector probe comprises the nucleotide sequence of SEQ ID NO: 108 or SEQ ID NO: 109 or a combination thereof. In addition the at least one hybridization probe comprises the nucleotide sequences of SEQ ID NO: 107.

In both embodiments and aspects thereof the combined isothermal reverse transcription, RNAse H and isothermal amplification reaction is performed on the nucleic acids as described supra to generate the plurality of single stranded RNA amplicons. Particularly, the temperature at which the isothermal reverse transcription and the isothermal amplification are performed, the promoter sequence and the RNA polymerase are as described supra. Also in both embodiments and aspects thereof the RNA amplicons hybridizing step and ambient temperature at which it is performed, the microarray and the attachment position of the hybridization probes thereon are as described supra. In addition the sample may comprise at least one of a nasopharyngeal swab, a nasal swab, a mouth swab, a skin swab and vaginal swab, a mouth wash, a skin wash, a plant wash, a homogenized food sample, a blood sample, a biopsy sample, an aerosol, or a hard surface swab.

In yet another embodiment of the present invention there is provided a method for identifying antibiotic resistance in a subject in need thereof, comprising obtaining a whole blood sample from the subject; extracting RNA from the whole blood sample; performing, on the RNA, a multiplexed, isothermal nucleic acid sequence-based amplification (NASBA) reaction to generate a plurality of RNA amplicons using at least one forward isothermal primer and at least one reverse isothermal primer each comprising nucleotide sequences specific to an antibiotic resistance determinant in a blood-borne pathogenic bacteria and specific to a detector probe binding site and on each reverse isothermal primer a T7 promoter sequence; hybridizing, at room temperature, the plurality of RNA amplicons to a plurality of labeled universal detector probes and to a plurality of hybridization probes each comprising a nucleotide sequence complementary to the nucleotide sequence specific to the antibiotic resistance determinant in the blood-borne pathogenic bacteria; said hybridization probes attached to a solid substrate on a microarray via an oligonucleotide linker to form a three dimensional structure thereon; washing the microarray at least once; and imaging the microarray to detect at least one signal from at least one of the plurality of labeled universal detector probes, thereby identifying antibiotic resistance in the subject.

In this embodiment the labeled universal detector probes may comprise a fluorescent label. Also in this embodiment the method may be performed with a clinical level of detection of 1 CFU/ml to about 10 CFU/ml. In addition, the blood-borne pathogenic bacteria may be Enterococcus faecium, Staphylococcus aureus, Klebsiella pneumoniae, Acinetobacter baumannii, Pseudomonas aeruginosa, Enterobacter cloacae, Enterobacter asburiae, Enterobacter hormaechei, Escherichia coli, or Klebsiella aerogenes. Further in this embodiment the isothermal primer pairs may target a 16S rRNA hypervariable 3 region or a 16S rRNA hypervariable 6 region or a combination thereof. In an aspect thereof the forward isothermal primer and the reverse isothermal primer targeting the 16S rRNA hypervariable 3 (HV3) region may comprise nucleotide sequences of SEQ ID NO: 117-118 or the forward isothermal primer and the reverse isothermal primer targeting the 16S rRNA hypervariable 6 (HV6) region may comprise nucleotide sequences of SEQ ID NO: 119-120. In addition the antibiotic resistance determinant in the blood-borne pathogenic bacteria may be an imp-1 gene, a kpc gene, an oxa-48 gene, a ndm-1 gene, a vim gene, a mcr-1 gene, a ctx-m gene, a mecA gene, a mecC gene, a vanA gene, or a vanB gene.

In this embodiment and aspect thereof the forward isothermal primer and the reverse isothermal primer amplifying the specific antibiotic resistance determinant sequence may comprise at least one of the group consisting of SEQ ID NOS: 121-122, SEQ ID NOS: 123-124, SEQ ID NOS: 125-126, SEQ ID NOS: 127-128, SEQ ID NOS: 129-130, SEQ ID NOS: 131-132, SEQ ID NOS: 133-134, SEQ ID NOS: 135-136, SEQ ID NOS: 137-138, SEQ ID NOS: 139-140, and SEQ ID NOS: 141-142. Also the plurality of hybridization probes may comprise at least one nucleotide sequence from each of an HV3a region, an HV3c region, an HV3e region, an HV6a region, an HV6c region and an HV6g region and at least one nucleotide sequence from the antibiotic resistance determinants. Particularly the at least one nucleotide sequence from the HV3a region may be selected from the group consisting of SEQ ID NOS: 145 to 156; wherein the at least one nucleotide sequence from the HV3c region may be selected from the group consisting of SEQ ID NOS: 157 to 165; wherein the at least one nucleotide sequence from the HV3e region may be selected from the group consisting of SEQ ID NOS: 166 to 170; wherein the at least one nucleotide sequence from the HV6a region may be selected from the group consisting of SEQ ID NOS: 171 to 176; wherein the at least one nucleotide sequence from the HV6c region may be selected from the group consisting of SEQ ID NOS: 177 to 185; wherein the at least one nucleotide sequence from the HV6g region may be selected from the group consisting of SEQ ID NOS: 186 to 192; and wherein the at least one nucleotide sequence from the antibiotic resistance determinants may be selected from the group consisting of SEQ ID NOS: 195 to 206.

In yet another embodiment of the present invention there is provided a method for detecting drug resistance in a blood-borne pathogenic bacteria, comprising obtaining a whole blood sample from a subject with an infection caused by the blood-borne pathogenic bacteria; extracting RNA from the whole blood sample; performing, on the RNA, a multiplexed, isothermal nucleic acid sequence-based amplification (NASBA) reaction to generate a plurality of RNA amplicons using at least one isothermal primer pair, said isothermal primer pairs comprising a forward isothermal primer and a reverse isothermal primer each of which comprise nucleotide sequences specific to a drug resistance marker in the blood borne pathogenic bacteria and to a detector probe binding site, each of said reverse isothermal primer further comprising a T7 promoter sequence; hybridizing, at room temperature, the plurality of RNA amplicons to a plurality of fluorescent labeled universal detector probes and to a plurality of hybridization probes each comprising a nucleotide sequence complementary to the nucleotide sequence specific to the antibiotic resistance genetic marker; said hybridization probes attached to a solid substrate on a microarray via an oligonucleotide linker to form a three dimensional structure thereon; washing the microarray at room temperature at least once; and imaging the microarray to detect at least one signal from at least one of the plurality of labeled universal detector probes, thereby detecting drug resistance in the blood borne pathogenic bacteria.

In this embodiment the method may be performed with a clinical level of detection as described supra. Also in this embodiment the blood-borne pathogenic bacteria may be as described supra. In addition the imp-1 gene, the ndm-1 gene, the kpc gene, and the ctx-m gene and the kpc gene, the oxa-48 gene, the vim gene, the mcr-1 gene, the ctx-m gene may be markers for resistance to carbapenem in Acinetobacter and in Enterobacteriaceae, respectively; wherein the vanA gene and the vanB gene may be markers for resistance to vancomycin in Enterococci; wherein the mecA gene and the mecC gene may be markers for resistance to methicillin in Staphylococcus aureus; wherein the kpc gene, the vim gene and the ctx-m gene are markers for multi-drug resistance in Pseudomonas aeruginosa; and wherein the ctx-m gene and the mcr-1 gene are markers for drug resistance in extended spectrum beta-lactamase (ESBL) producing Enterobacteriaceae.

In this embodiment the isothermal primer pairs may target a 16S rRNA hypervariable 3 region or a 16S rRNA hypervariable 6 region or a combination thereof. Particularly, the isothermal primer pair targeting the 16S rRNA hypervariable 3 (HV3) region may comprise nucleotide sequences of SEQ ID NO: 117-118 or the isothermal primer pair targeting the 16S rRNA hypervariable 6 (HV6) region may comprise nucleotide sequences of SEQ ID NO: 119-120. Also the isothermal primer pairs amplifying the specific drug resistance marker sequence may comprise at least one of the group consisting of SEQ ID NOS: 121-122, SEQ ID NOS: 123-124, SEQ ID NOS: 125-126, SEQ ID NOS: 127-128, SEQ ID NOS: 129-130, SEQ ID NOS: 131-132, SEQ ID NOS: 133-134, SEQ ID NOS: 135-136, SEQ ID NOS: 137-138, SEQ ID NOS: 139-140, and SEQ ID NOS: 141-142. In addition the plurality of hybridization probes may comprise at least one nucleotide sequence from each of an HV3a region, an HV3c region, an HV3e region, an HV6a region, an HV6c region and an HV6g region and at least one nucleotide sequence from the drug resistance markers. Particularly, the at least one nucleotide sequence from the HV3a region, from the HV3c region, from the HV3e region, from the HV6a region, from the HV6c region, and from the HV6g region are as described supra.

Provided herein is a multiplex isothermal NASBA amplification method for testing for the presence of antibiotic resistance determinants in pathogenic bacterial targets, such as, but not limited to, ESKAPE+ pathogens. In a non-limiting example, the method identifies antibiotic-resistance determinants that confer resistance to carbapenamases (imp-1, kpc, oxa-48, ndm-1, and vim), colistin (mcr-1), extended spectrum beta lactamases (ctx-m), methicillin (mecA, mecC), and vancomycin (vanA, vanB) that pose an urgent or serious threat as per the CDC (Table 11). Identification of the ESKAPE+ pathogens utilizes amplification of 16S rRNA, such as from the hypervariable 3 and/or 6 regions (HV3, HV6) and coincident hybridization to multiple 16S rRNA targets/probes. Primers and probes are shown in Tables 12 and 13 (Example 5).

TABLE 11
Priority blood-borne pathogens and
antibiotic-resistance determinants

| Microorganism | Threat | Genetic Marker(s) |
|---|---|---|
| Carbapenem-resistant | Urgent | imp-1, ndm-1, |
| Acinetobacter (including | | kpc, ctx-M |
| species-level identification | | |
| of A. baumannii) | | |
| Carbapenem-resistant | Urgent | kpc, vim, ctx-M, |
| Enterobacteriaceae | | oxa-48, mcr-1 |
| Vancomycin-resistant | Serious | vanA, vanB |
| Enterococci (including | | |
| species-level | | |
| identification of E. faecium) | | |
| Methicillin-resistant | Serious | mecA, mecC |
| Staphylococcus aureus | | |
| Multi-drug resistant | Serious | kpc, vim, ctx-M |
| Pseudomonas aeruginosa | | |
| ESBL-producing | Serious | ctx-M, mcr-1 |
| Enterobacteriaceae (including | | |
| species-level identification | | |
| of E. coli) | | |

Generally, the methods provided herein utilize multiplexed NASBA chemistry in an isothermal 13-gene ESKAPE+ drug susceptibility test for whole blood. Gene-specific primers to initiate a reverse transcriptase reaction from bacterial RNA, while amplification occurs under the direction of a T7 RNA polymerase promoter. Amplified RNA transcripts are then detected on a PDx microarray. This novel approach overcomes primer artifacts encountered with conventional multiplexed amplification techniques, and improves upon current analytical and clinical sensitivity because multi-copy RNAs instead of single-copy DNAs are amplified. Detecting RNA instead of DNA also provides increased clinical confidence that a positive test is indicative of an active infection.

The multiplex isothermal NASBA amplification and microarray identification assay may be performed on an automated sample-to-answer, integrated microarray system which addresses the deficiencies in standard platforms. Sample-to-answer detection is performed with a clinical level of detection (LoD) of about 1-10 CFU/mL, greater than 95% clinical sensitivity and specificity for microbial identification and resistance gene detection and a sample-to-answer turnaround time of less than 3 hours. It is contemplated that automating this isothermal, multiplexed amplification chemistry may relieve a universal bottleneck in infectious disease diagnostics and molecular drug susceptibility testing and extend to new resistance signatures as pathogens adapt to new environments and drugs.

Also provided is a multiplex isothermal TMA or NASBA amplification method of several viral RNA targets and a control human RNA target concurrently followed by hybridization of those ribonucleic amplicons to surface bound nucleic acid probes comprising a microarray. Moreover, subsequent to such multiplex isothermal RNA amplification, the resulting microarray hybridization may be used to identify the presence of each individual viral RNA target in the original sample, as distinct from the human RNA control, and, where appropriate, to obtain sequence information at discrete locations within one or more of those RNA targets. The resulting local sequence information may be used to detect local genetic variation within a RNA sequence, which, in the case of viral RNA sequences, may be used to detect the presence of specific viral sequence variation which can be used to identify specific viral sub-types (i.e. variants). Particularly, a mixture of the RNA viruses SARS-COV-2, Influenza A and Influenza B and also a human RNA amplification control (B2M) was detected where additional sequencing by hybridization was performed on a specific region of the SARS-COV-2 genome, which in turn is used to establish which SARS-COV-2 Variant is present in the viral mixture.

During the multiplex TMA or NASBA reaction, primer mediated isothermal amplification produces RNA amplicons which acquire Detector Probe Binding sequences which are suitable to bind fluorescently labeled Detector Probes that are introduced in the hybridization or wash buffers, thereby forming a [Hybridization Probe-RNA amplicon-Detector Probe] “Sandwich”. In the present Example, the Detector Probes are DNA oligonucleotides synthesized with a biotin group at their 5′ termini. When mixed with Streptavidin modified Phycoerythrin (SAPE) a SAPE-Detector Probe complex is formed, thereby labeling the RNA amplicon as bound to its cognate hybridization probe.

The following examples are given for the purpose of illustrating various embodiments of the invention and are not meant to limit the present invention in any fashion.

### EXAMPLE 1

### Method

### Transcription Mediated Amplification

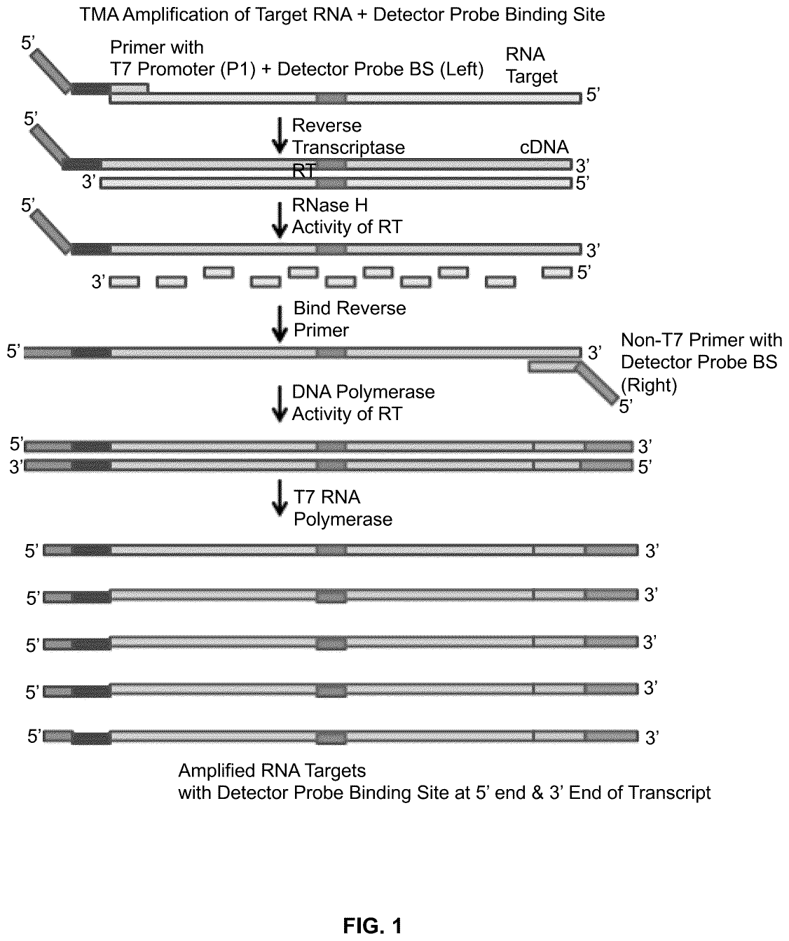

FIG. 1 illustrates the general approach to amplifying a target RNA using Transcription Mediated Amplification (TMA), or alternatively the corresponding NASBA variant of TMA that is in every way identical, except that an exogenous RNAseH enzyme is added to the enzymatic reaction rather than using the endogenous RNAseH activity of Reverse Transcriptase. The TMA (NASBA) method produces an amplified RNA that is modified during the course of the combined “One Pot” enzyme reactions to include a new terminal RNA sequence (“Primer with T7 promoter”, P1) or in some cases a new T7 promoter adjacent to a detector probe nucleotide sequence, either introduced as a DNA oligonucleotide primer during reverse transcription. The cDNA generated after reverse transcription is amplified using a non-T7 primer with a detector probe nucleotide sequence (15-30 bases) attached at its 5′ end (FIG. 1). After completion of the combined reverse transcription/amplification reaction, the resulting double stranded cDNA is incubated with T7 RNA polymerase to generate multiple copies of single stranded RNA amplicons (FIG. 1).

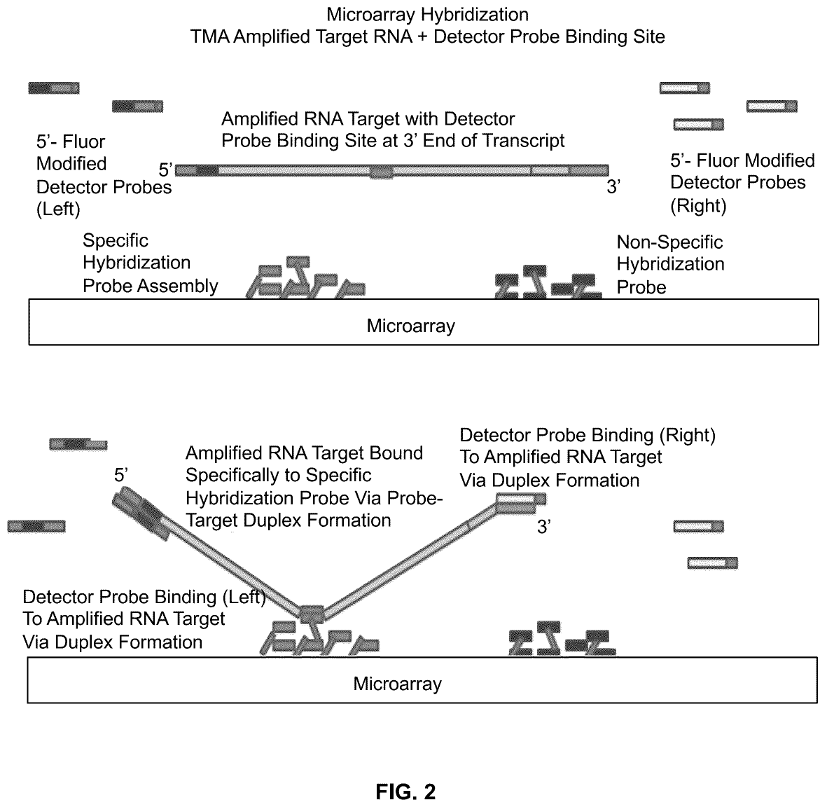

In the present invention, surface bound oligonucleotide hybridization probes are designed with a sequence that binds to amplified RNA (FIG. 2) at ambient temperature (15° C.-30° C.). The probes are fabricated on the microarray surface such that they are positioned away from direct contact with the underlying microarray substrate, preferably in a 3-dimensional assembly formed as shown in FIG. 2. Thus, when TMA (NASBA) amplified RNA binds to its cognate hybridization probe at ambient temperature, the bound complex is also positioned away from the microarray substrate. Upon binding of the detector probes which had been introduced as part of the hybridization or washing reactions, the 3-fold complex (hybridization probe+amplified RNA target+detector probe) comprises a “Sandwich Assay” analogous to immunoassays. The dye or chemical label introduced onto the detector probe can be for example, a fluorescent tag chemically linked to the 5′ end of the detector probe or a conjugate such as phycoerthyrin-streptaviden bound non-covalently to a biotin moiety covalently linked to the 5′ end of the detector probe. Either labeling approach (covalent or non-covalent) thus enabling detection of amplified RNA binding to hybridization probes at the surface.

### Multiplex Analysis of Viral Contamination

A sample containing one or more viruses is collected from a human or animal subject from the airway, via a Nasopharyngeal (NP) or Anterior Nasal (AN) swab, or from saliva. The sample is dispersed in a saline solution and lysed by heating. RNA is extracted by magnetic beads or similar methods. Alternatively, the sample is analyzed directly without RNA extraction.

Concentrated TMA enzyme solution is added to the extracted RNA or lysed solution and amplified as a two-step isothermal reaction, once at 65° C. for about 5 min and then once at 41° C. for about 10 min to about 60 min to produce “n” different amplified RNAs, to be analyzed, as one set on a DNA microarray hybridization device. The amplified product is used as-is by adding a binding buffer containing a single detector probe, at roughly an equimolar concentration relative to that of the RNA amplicon product and suitable to bind to all (n) amplified viral RNAs concurrently. In a preferred implementation, the resulting solution is then applied directly to a single microarray, to allow hybridization at or near room temperature (15° C.-30° C.). The microarray presents at least (n) hybridization probes specific for one or more sequences within each of the (n) TMA-amplified viral RNAs. Analysis of the resulting microarray hybridization pattern is used to detect which among a panel of several viral candidates is present in the sample. Alternatively, within a single virus, probes are chosen which can distinguish wild type sequences from mutant sequences in the virus among at least (n) such sites of the RNA genome. When performed in this manner, the distribution among viruses in a test panel, or the identification of mutations within a virus can be assayed at high throughput in a regional laboratory or at point of collection as in a doctor's office, clinic, or related simple public health venue.

### Multiplex Analysis of Bacterial Contamination

A sample containing one or more bacteria is collected from a human or animal subject or from a plant or from a surface, via a swab or from a liquid rinse of the human or animal or surface material under study. The swab or liquid rinse is then dispersed in a saline solution and lysed by heating. RNA is then be extracted by magnetic beads or similar methods. Alternatively, the sample is analyzed directly without RNA extraction.

Concentrated TMA (NASBA) enzyme solution is added to the extracted RNA or lysed solution and then amplified in a two-step isothermal reaction at 65° C. then 41° C. as described above to produce (n) different amplified bacterial RNAs. The product is then used as-is by adding a binding buffer and a plurality of a single or a pair of detector probe nucleotide sequence suitable to bind to all (n) amplified bacterial RNAs, concurrently. In a preferred implementation, the resulting solution is then applied directly to a single microarray, to allow hybridization at or near room temperature (15° C.-30° C). The microarray presents at least (n) hybridization probes specific for one or more sequences within in each of the (n) TMA (NASBA) amplified bacterial RNAs. Analysis of the resulting microarray hybridization pattern is used to detect which, among a panel of several bacterial candidates, is present in the sample. Alternatively, within a single bacterium, probes are chosen which can distinguish wild type from mutant sequences in the bacterium among at least (n) such sites or to identify markers in a bacterium to identify drug resistance. When performed in this manner, the distribution among bacteria in a test panel, or the identification of mutations within a bacterium or drug resistance markers present in the bacterium can be assayed at high throughput in a regional laboratory or at point of collection as in a doctor's office, clinic, or related simple public health venue.

### Multiplex Analysis of Cancer Biomarkers, Mutation, Deletion, Spice Variation on Single Cells

A sample containing one or more tumor cells is collected from a human or animal subject, via tissue dissection then dispersion of the tissue, or from a fluid phase tumor cell suspension such as a lymphoid tumor or blood borne metastatic tumor cell. In some instances, the dispersed tumor cells are individually isolated to be used for “Single Cell Analysis”. The resulting dispersed tumor cells are then lysed by heating. The RNA from those lysed cells may then be extracted by magnetic beads or similar methods. Alternatively, the sample is analyzed directly without RNA extraction, processing as a very small volume sample (1 μL-10 μL).

Concentrated TMA (NASBA) enzyme solution is then added to the extracted RNA or lysed solution and then amplified as a two-step isothermal reaction once at 65° C. then once at 41° C. to produce (n) different amplified human RNAs bearing meaningful tumor related markers. Such markers may include a panel of local mutational changes to be used for risk prediction or diagnosis or response to treatment. The panel may also include mRNA splice variants useful for tumor analysis or may include RNA transcripts which are greatly over-expressed in the tumor or a tumor related cell type. Subsequent to TMA (NASBA), the product is then used as-is by adding a binding buffer containing a plurality of a single detector probe nucleotide sequence suitable to bind to all (n) amplified tumor cell RNAs, concurrently. The resulting solution is then applied directly to a single Microarray which bears at least (n) hybridization probes specific for one or more sequences in each of the (n) amplified tumor cell RNAs, especially those suitable to detect mutation or deletion or spice variation or the presence of one or more over-expressed mRNA transcripts. Analysis of the resulting Microarray Hybridization pattern is used to detect, among a panel of (n) tumor related genetic changes or splice variations or mRNA species over-expressed in a cell, which of those changes is present in the tumor cell sample under study. When performed in that way, the distribution of mutations or spice variation or greatly altered mRNA transcription can be assayed at high throughput in a regional laboratory or at point of collection as in a doctor's office, clinic, or related simple public health venue.

### DetectX-PoCv assay

The DetectX-PoCv assay is a Point of Care multiplex assay that can test for all variants of Concern and Variants of Interest of a virus in a single reaction at single nucleotide resolution in five (5) simple and easy steps from sample to answer with results in less than 1 hour. A DetectX-PoCv kit, a mini-incubator, and portable handheld sized imager is all that is required to conduct the test. No additional consumables are required.
1. A sample is collected from a dry swab and is fed into an Isothermal reaction tube with an eye dropper. The tube is allowed to incubate for 15 minutes.
2. One drop (1) of Hybridization buffer is then added into the tube, and then closed on the eye-dropper end. The tube is then mixed by swirling.
3. From the eye-dropper end of the tube, four (4) drops of reaction solution are then squeezed into the entry port of a single-glass slide chiplet-microarray. The reaction solution is allowed to hybridize the probes in the chiplet-microarray for 15 minutes.
4. Then four (4) drops of wash solution are then added from the tube, by squeezing the tube into the entry port of the single-glass slide chiplet-microarray. The wash solution is then allowed to sit for 10 minutes, and then drains into the assay fluids collection chamber.
5. The glass slide is inserted into a portable, hand-held imager using the alignment tabs, and Augury software is processed for imaging, results and analysis.

Particularly, DetectX-PoCv assay is a rapid molecular in vitro diagnostic test utilizing an multiplex isothermal nucleic acid amplification technology intended for the qualitative detection of nucleic acid from the SARS-COV-2 viral RNA and Variants of Concern (VoC)/Variants of Interest (Vol) mutation detection in direct nasal, nasopharyngeal or throat swabs from individuals who are suspected of COVID-19 by their healthcare provider within the first seven days of the onset of symptoms. Results for the identification of SARS-COV-2 RNA and VoC/Vol mutations are:
1. Covid-19: Measured via Mutation independent Universal Probes
2. Covid-19 Variants of Concern: Alpha-B.1.1.7-U.K, Beta B.1.351-S. Africa, Epsilon-B.1.427-US-California, Epsilon-B.1.429-US-California, Gamma-P.1 Japan/Brazil
3. Covid-19 Variants of Interest: Eta-B.1.525-U.K/Nigeria, lota-B.1.526-US-New York, B.1.526.1-US-New York, Zeta-P.2-Brazil, B.1.2 SARS-COV-2

The SARS-COV-2 RNA and VoC/Vol mutations are generally detectable in respiratory samples during the acute phase of infection. Positive results are indicative of the presence of SARS-COV-2 RNA and Variants; clinical correlation with patient history and other diagnostic information is necessary to determine patient infection status. Positive results do not rule out bacterial infection or co-infection with other viruses. Testing facilities within the United States and its territories are required to report all results to the appropriate public health authorities. Negative results should be treated as presumptive and, if inconsistent with clinical signs and symptoms or necessary for patient management, should be tested with different authorized or cleared molecular tests. Negative results do not preclude SARS-COV-2 infection and should not be used as the sole basis for patient management decisions. Negative results should be considered in the context of a patient's recent exposures, history and the presence of clinical signs and symptoms consistent with COVID-19.

### EXAMPLE 2

### Multiplex Analysis of Viral Contamination

From December 2019 to May 2021, as the COVID-19 virus spread among multiple nations, it developed multiple stable mutations, especially in its Spike gene, which conferred a selective advantage, enhancing infectivity, thereby enhancing the rate of spread of these variants. In the subsequent 18 months, multiple stable Spike mutations of the virus became evident. It is well known in the art that DNA microarray technology can be used to detect mutations, especially deletions and single nucleotide polymorphism (SNP) of the type seen in the COVID-19 Spike gene. The location of (n) such Spike mutations in the COVID-19 RNA genome is displayed in Table 1. Each of the (n) columns in Table 1 correspond to the location of a specific Spike mutation. To the left of the Table is a list of COVID-19 variants. Inspection of each row of identifies the specific pattern of mutation among the potential set of (n) mutations which can identify each COVID-19 variant. At the bottom of Table 1 is a series of (8) horizontal boxes that identify the position of domains within the Spike gene that can be amplified by RT-PCR. Upon amplification of one or more of those regions to generate a DNA amplimer fragment, the resulting DNA amplimer possesses a DNA sequence capable of binding to a hybridization probe corresponding to each site of potential Spike gene mutation.

TABLE 1
Layout of Hybridization Probes and Primers for Multiplex Analysis of COVID-19 Variants

| Spike Gene Target Region (Codon) Amino | | | | | | | | | | | | | | | | | |
|---|---|---|---|---|---|---|---|---|---|---|---|---|---|---|---|---|---|
| Acid Change | | | | | | | | | | | | | | | | | |
| | | | CDC % | | | | | | | | | | | | | | |
| | | | (Mar. | Incidence % | | | | | | | | | | | | | |
| | | | 14-27 | (Gisaid | | | | L18F | | T20N | P26S | Q52R | A67V | Δ69-70 | | D80A/G | |
| | Street | Pango | 2021, | March | L5F | | S13I | S1 subunit (14-685) | | | | | | | | | |
| | name | lineage | US) | 2021) | Signal (1-13) | | | N-terminal domain (14-305) | | | | | | | | | |
| VOC | UK | B.1.1.7 | 44.10% | 49.81% | L³ | | S² | L³ | | T² | P³ | Q³ | A³ | Δ¹ | | D² | |
| VOC | Cal | B.1.429 | 6.90% | 2.08% | L³ | | I¹ | L³ | | T² | P³ | Q³ | A³ | HV² | | D² | |
| | L452R | | | | | | | | | | | | | | | | |
| VOC | | B.1.427 | 2.90% | 0.90% | L³ | | S/I¹ | L³ | | T² | P³ | Q³ | A³ | HV² | | D² | |
| VOC | Brazil | P.1 | 1.40% | 0.39% | L³ | | S² | F | | N¹ | S | Q³ | A³ | HV | | D² | |
| VOC | SA | B.1.351 | 0.70% | 1.13% | L³ | | S² | L³ | | T² | P³ | Q³ | A³ | HV | | A¹ | |
| VOI | NYC | B.1.526 | 9.20% | 0.82% | L/F | | S² | L³ | | T² | P³ | Q³ | A³ | HV² | | D² | |
| | (Ho | | | | | | | | | | | | | | | | |
| | et al.) | | | | | | | | | | | | | | | | |
| VOI | NYC | B.1.525 | 0.50% | 0.10% | L³ | | S² | L³ | | T² | P³ | R | V | Δ¹ | | D² | |
| VOI | Rio | P.2 | 0.30% | 0.36% | L³ | | S² | L³ | | T² | P³ | Q³ | A³ | HV² | | D² | |
| | de Jan. | | | | | | | | | | | | | | | | |
| | | B.1.2 | 10% | 7.83% | L³ | | S² | L³ | | T² | P³ | Q³ | A³ | HV² | | D² | |
| | | B.1, B.1.1, | 2.4%/ | 2.6%/ | L³ | | S² | L³ | | T² | P³ | Q³ | A³ | HV² | | D² | |
| | | B.1.234 | 0.9%/ | 1.5%/ | | | | | | | | | | | | | |
| | | | 0.5% | 0.5% | | | | | | | | | | | | | |
| | | B.1.1.519 | 4.10% | 1.50% | L³ | | S² | L³ | | T² | P³ | Q³ | A³ | HV² | | D² | |
| | | B.1.526.1 | 3.90% | 0.35% | F | | S² | L³ | | T² | P³ | Q³ | A³ | HV² | | G | |
| | | B.1.526.2 | 2.90% | 0.18% | F | | S² | L³ | | T² | P³ | Q³ | A³ | HV² | | D² | |
| | | B.1.596 | 1.70% | 1.04% | L³ | | S² | L³ | | T² | P³ | Q³ | A³ | HV² | | D² | |
| | | R.1 | 1.20% | 0.20% | L³ | | S² | L³ | | T² | P³ | Q³ | A³ | HV² | | D² | |
| | | B.1.575 | 1.10% | 0.19% | L³ | | S² | L³ | | T² | P³ | Q³ | A³ | HV² | | D² | |
| | | B.1.243, | 0.60% | 0.84% | L³ | | S² | L³ | | T² | P³ | Q³ | A³ | HV² | | D² | |
| | | B.1.1.207 | | | | | | | | | | | | | | | |
| | US | B.1.375 | <1% | 0.03% | L³ | | S² | L³ | | T² | P³ | Q³ | A³ | Δ¹ | | D² | |
| | B.1.1.1, B.1.416, | | <1% | 0.50% | L³ | | S² | L³ | | T² | P³ | Q³ | A³ | HV² | | D | |
| | B.1.1.33, B.1.311, | | | | | | | | | | | | | | | | |
| | B.1.1.122 | | | | | | | | | | | | | | | | |
| | Brazil | B.1.1.28 | <1% | 0.10% | L³ | | S² | L³ | | T² | P³ | Q³ | A³ | HV² | | D | |
| | (ORIG) | | | | | | | | | | | | | | | | |
| | Andrah | B.1.1.420 | <1% | 0.08% | L³ | | S² | F | | T² | P³ | Q³ | A³ | HV² | | D | |
| | Pradesh | | | | | | | | | | | | | | | | |
| | | A.23.1 | <1% | 0.05% | L³ | | S² | L³ | | T² | P³ | Q³ | A³ | HV² | | D | |
| | | A.27 | <1% | 0.05% | L³ | | S² | F | | T² | P³ | Q³ | A³ | HV² | | D | |
| | | A.28 | <1% | 0.02% | L³ | | S² | L³ | | T² | P³ | Q³ | A³ | Δ¹ | | D | |
| | Mink/ | B.1.1.298 | <1% | 0.00% | L³ | | S² | L³ | | T² | P³ | Q³ | A³ | Δ¹ | | D | |
| | Cluster V | | | | | | | | | | | | | | | | |
| | | B.1.1.318 | <1% | 0.01% | L³ | | S² | L³ | | T² | P³ | Q³ | A³ | HV² | | D | |
| | | B.1.160 | <1% | 1.76% | L³ | | S² | L³ | | T² | P³ | Q³ | A³ | HV² | | D | |
| | | B.1.177 | <1% | 3.19% | L³ | | S² | F | | T² | P³ | Q³ | A³ | HV² | | D | |
| | | B.1.177.80 | <1% | 0.04% | L³ | | S² | F | | T² | P³ | Q³ | A³ | HV² | | D | |
| | | B.1.258 | <1% | 1.15% | L³ | | S² | L³ | | T² | P³ | Q³ | A³ | HV/Δ¹ | | D | |
| | | B.1.258.14 | <1% | 0.06% | L³ | | S² | L³ | | T² | P³ | Q³ | A³ | HV² | | D | |
| | | B.1.258.17 | <1% | 1.02% | L³ | | S² | L³ | | T² | P³ | Q³ | A³ | Δ¹ | | D | |
| | | B.1.517 | <1% | 0.25% | L³ | | S² | L³ | | T² | P³ | Q³ | A³ | HV² | | D | |
| | WUHAN | WUHAN | — | — | L³ | | S² | L³ | | T² | P³ | Q³ | A³ | HV² | | D | |
| PCR Amplimer length (bases) | | | | | (1) 101 | | | | | | | | (2B) 150 | | | | |
| | | | T95I | D138Y | Y144DEL | | W152C | F157L/S | | L189F | R190S | D215G | A222V | A243del | G252V | | D253G |
| | Street | Pango | S1 subunit (14-685) | | | | | | | | | | | | | | |
| | name | lineage | N-terminal domain (14-305) | | | | | | | | | | | | | | |
| VOC | UK | B.1.1.7 | T³ | D² | Δ¹ | | W² | F³ | | L³ | R³ | D³ | A³ | A² | G³ | | D² |
| VOC | Cal | B.1.429 | T³ | D² | Y² | | C¹ | F³ | | L³ | R³ | D³ | A³ | A² | G³ | | D² |
| | L452R | | | | | | | | | | | | | | | | |
| VOC | | B.1.427 | T³ | D² | Y² | | W/C¹ | F³ | | L³ | R³ | D³ | A³ | A² | G³ | | D² |
| VOC | Brazil | P.1 | T³ | Y¹ | Y² | | W² | F³ | | L³ | S | D³ | A³ | A² | G³ | | D² |
| VOC | SA | B.1.351 | T³ | D² | Y² | | W² | F³ | | L³ | R³ | G | A³ | Δ¹ | G³ | | D² |
| VOI | NYC | B.1.526 | I | D² | Y² | | W² | F³ | | L³ | R³ | D³ | A³ | A² | G³ | | G¹ |
| | (Ho | | | | | | | | | | | | | | | | |
| | et al.) | | | | | | | | | | | | | | | | |
| VOI | NYC | B.1.525 | T³ | D² | Δ¹ | | W² | F³ | | L³ | R³ | D³ | A³ | A² | G³ | | D² |
| VOI | Rio | P.2 | T³ | D² | Y² | | W² | F³ | | L³ | R³ | D³ | A³ | A² | G³ | | D² |
| | de Jan. | | | | | | | | | | | | | | | | |
| | | B.1.2 | T³ | D² | Y² | | W² | F³ | | L³ | R³ | D³ | A³ | A² | G³ | | D² |
| | | B.1, B.1.1, | T³ | D² | Y² | | W² | F³ | | L³ | R³ | D³ | A³ | A² | G³ | | D² |
| | | B.1.234 | | | | | | | | | | | | | | | |
| | | B.1.1.519 | T³ | D² | Y² | | W² | F³ | | L³ | R³ | D³ | A³ | A² | G³ | | D² |
| | | B.1.526.1 | I | D² | Δ¹ | | W² | S | | L³ | R³ | D³ | A³ | A² | G³ | | D/G¹ |
| | | B.1.526.2 | T³ | D² | Y² | | W² | F³ | | L³ | R³ | D³ | A³ | A² | G³ | | G¹ |
| | | B.1.596 | T³ | D² | Y² | | W² | F³ | | L³ | R³ | D³ | A³ | A² | G³ | | D² |
| | | R.1 | T³ | D² | Y² | | L | F³ | | L³ | R³ | D³ | A³ | A² | G³ | | D² |
| | | B.1.575 | T³ | D² | Y² | | W² | F³ | | L³ | R³ | D³ | A³ | A² | G³ | | D² |
| | | B.1.243, | T³ | D² | Y² | | W² | F³ | | L³ | R³ | D³ | A³ | A² | G³ | | D² |
| | | B.1.1.207 | | | | | | | | | | | | | | | |
| | US | B.1.375 | T³ | D² | Y² | | W² | F³ | | L³ | R³ | D³ | A³ | A² | G³ | | D² |
| | B.1.1.1, B.1.416, | | T³ | D² | Y² | | W² | F³ | | L³ | R³ | D³ | A³ | A² | G³ | | D² |
| | B.1.1.33, B.1.311, | | | | | | | | | | | | | | | | |
| | B.1.1.122 | | T³ | D² | Y² | | W² | F³ | | L³ | R³ | D³ | A³ | A² | G³ | | D² |
| | Brazil | B.1.1.28 | T³ | D² | Y² | | W² | F³ | | L³ | R³ | D³ | A³ | A² | G³ | | D² |
| | (ORIG) | | | | | | | | | | | | | | | | |
| | Andrah | B.1.1.420 | T³ | D² | Y² | | W² | F³ | | L³ | R³ | D³ | A³ | A² | G³ | | D² |
| | Pradesh | | | | | | | | | | | | | | | | |
| | | A.23.1 | T³ | D² | Y² | | W² | L | | L³ | R³ | D³ | A³ | A² | G³ | | D² |
| | | A.27 | T³ | D² | Y² | | W² | F³ | | L³ | R³ | D³ | A³ | A² | G³ | | D² |
| | | A.28 | T³ | D² | Y² | | W² | F³ | | L³ | R³ | D³ | A³ | A² | G³ | | D² |
| | Mink/ | B.1.1.298 | T³ | D² | Y² | | W² | F³ | | L³ | R³ | D³ | A³ | A² | G³ | | D² |
| | Cluster V | | | | | | | | | | | | | | | | |
| | | B.1.1.318 | T³ | D² | Δ¹ | | W² | F³ | | L³ | R³ | D³ | A³ | A² | G³ | | D² |
| | | B.1.160 | T³ | D² | Y² | | W² | F³ | | L³ | R³ | D³ | A³ | A² | G³ | | D² |
| | | B.1.177 | T³ | D² | Y² | | W² | F³ | | L³ | R³ | D³ | V | A² | G³ | | D² |
| | | B.1.177.80 | T³ | D² | Δ/Y¹ | | W² | F³ | | L³ | R³ | D³ | V | A² | G³ | | D² |
| | | B.1.258 | T³ | D² | Y² | | W² | F³ | | L³ | R³ | D³ | A³ | A² | G³ | | D² |
| | | B.1.258.14 | T³ | D² | Y² | | W² | F³ | | L³ | R³ | D³ | A³ | A² | G³ | | D² |
| | | B.1.258.17 | T³ | D² | Y² | | W² | F³ | | F | R³ | D³ | A³ | A² | G³ | | D² |
| | | B.1.517 | T³ | D² | Y² | | W² | F³ | | L³ | R³ | D³ | A³ | A² | G/V | | D² |
| | WUHAN | WUHAN | T³ | D² | Y² | | W² | F³ | | L³ | R³ | D³ | A³ | A² | G³ | | D² |
| PCR Amplimer length (bases) | | | | (3) 129 | | | | | | | | | | (4_{B}) 160 | | | |
| Spike Gene Target Region (Codon) Amino | | | | | | | | | | | | | | | | | |
| Acid Change | | | | | | | | | | | | | | | | | |
| | | | CDC % | | | | | | | | | | | | | | |
| | | | Mar. | Incidence % | | | | | | | | | | | | | |
| | | | 14-27 | Gisaid | | | | | | | | | | | | | |
| | Street | Pango | 2021 | March | V367F | K417N/T | N439K | N440K | L452R | Y453F | S477N | V483A | E484K | S494P | NS01Y/T | | A570D |
| | name | lineage | (US) | 2021 | RBD (319-541) | | | | | | | | | | | | |
| VOC | UK | B.1.1.7 | 44.10% | 49.81% | V³ | K² | N² | N² | L² | Y² | S² | V | E/K¹ | S/P | Y¹ | | D |
| VOC | Cal | B.1.429 | 6.90% | 2.08% | V³ | K² | N² | N² | R¹ | Y² | S² | V | E | S³ | N² | | A³ |
| | L452R | | | | | | | | | | | | | | | | |
| VOC | | B.1.427 | 2.90% | 0.90% | V³ | K² | N² | N² | R¹ | Y² | S² | V | E | S³ | N² | | A³ |
| VOC | Brazil | P.1 | 1.40% | 0.39% | V³ | N/T¹ | N² | N² | L² | Y² | S² | V | K¹ | S³ | Y¹ | | A³ |
| VOC | SA | B.1.351 | 0.70% | 1.13% | V³ | N¹ | N² | N² | L² | Y² | S² | V | K¹ | S³ | Y¹ | | A³ |
| VOI | NYC | B.1.526 | 9.20% | 0.82% | V³ | K² | N² | N² | L² | Y² | S/N¹ | V | E/K¹ | S³ | N² | | A³ |
| | (Ho | | | | | | | | | | | | | | | | |
| | etal) | | | | | | | | | | | | | | | | |
| VOI | NYC | B.1.525 | 0.50% | 0.10% | V³ | K² | N² | N² | L² | Y² | S² | V | K¹ | S³ | N² | | A³ |
| VOI | Rio | P.2 | 0.30% | 0.36% | V³ | K² | N² | N² | L² | Y² | S² | V | K¹ | S³ | N² | | A³ |
| | de Jan. | | | | | | | | | | | | | | | | |
| | | B.1.2 | 10% | 7.83% | V³ | K² | N² | N² | L² | Y² | S² | V | E | S³ | Y¹ | | A³ |
| | | B.1, B.1.1, | 2.4%/ | 2.6%/ | V³ | K² | N² | N² | L² | Y² | S² | V | E | S³ | N² | | A³ |
| | | B.1.234 | 0.9%/ | 1.5%/ | | | | | | | | | | | | | |
| | | | 0.5% | 0.5% | | | | | | | | | | | | | |
| | | B.1.1.519 | 4.10% | 1.50% | V³ | K² | N² | N² | L² | Y² | S² | V | E | S³ | N² | | A³ |
| | | B.1.526.1 | 3.90% | 0.35% | V³ | K² | N² | N² | R¹ | Y² | S² | V | K¹ | S³ | N² | | A³ |
| | | B.1.526.2 | 2.90% | 0.18% | V³ | K² | N² | N² | L² | Y² | N¹ | V | E | S³ | N² | | A³ |
| | | B.1.596 | 1.70% | 1.04% | V³ | K² | N² | N² | L² | Y² | S² | V | E | S³ | N² | | A³ |
| | | R.1 | 1.20% | 0.20% | V³ | K² | N² | N² | L² | Y² | S² | V | K¹ | S³ | N² | | A³ |
| | | B.1.575 | 1.10% | 0.19% | V³ | K² | N² | N² | L² | Y² | S² | V | E | S³ | N² | | A³ |
| | | B.1.243, | 0.60% | 0.84% | V³ | K² | N² | N² | L² | Y² | S² | V² | E² | S³ | N² | | A³ |
| | | B.1.1.207 | | | | | | | | | | | | | | | |
| | US | B.1.375 | <1% | 0.03% | V³ | K² | N² | N² | L² | Y² | S² | V² | E² | S³ | N² | | A³ |
| | B.1.1.1, B.1.416, | | <1% | 0.50% | V³ | K² | N² | N² | L² | Y² | S² | V² | E² | S³ | N² | | A³ |
| | B.1.1.33, B.1.311, | | | | | | | | | | | | | | | | |
| | B.1.1.122 | | | | | | | | | | | | | | | | |
| | Brazil | B.1.1.28 | <1% | 0.10% | V³ | K² | N² | N² | L² | Y² | S² | V² | E² | S³ | N² | | A³ |
| | (ORIG) | | | | | | | | | | | | | | | | |
| | Andrah | B.1.1.420 | <1% | 0.08% | V³ | K² | N² | K¹ | L² | Y² | S² | V² | E² | S³ | N² | | A³ |
| | Pradesh | | | | | | | | | | | | | | | | |
| | | A.23.1 | <1% | 0.05% | F | K² | N² | N² | L² | Y² | S² | V² | E/K¹ | S³ | N² | | A³ |
| | | A.27 | <1% | 0.05% | V³ | K² | N² | N² | R¹ | Y² | S² | V² | E² | S³ | Y¹ | | A³ |
| | | A.28 | <1% | 0.02% | V³ | K² | N² | N² | L² | Y² | S² | V² | E² | S³ | T | | A³ |
| | Mink/ | B.1.1.298 | <1% | 0.00% | V³ | K² | N² | N² | L² | F¹ | S² | V² | E² | S³ | N² | | A³ |
| | Cluster V | | | | | | | | | | | | | | | | |
| | | B.1.1.318 | <1% | 0.01% | V³ | K² | N² | N² | L² | Y² | S² | V² | K¹ | S³ | N² | | A³ |
| | | B.1.160 | <1% | 1.76% | V³ | K² | N² | N² | L² | Y² | N¹ | V² | E² | S³ | N² | | A³ |
| | | B.1.177 | <1% | 3.19% | V³ | K² | N² | N² | L² | Y² | S² | V² | E² | S³ | N² | | A³ |
| | | B.1.177.80 | <1% | 0.04% | V³ | K² | N² | N² | L² | Y² | S² | V² | E² | S³ | N² | | A³ |
| | | B.1.258 | <1% | 1.15% | V³ | K² | K¹ | N² | L² | Y² | S² | V² | E² | S³ | N² | | A³ |
| | | B.1.258.14 | <1% | 0.06% | V³ | K² | K¹ | N² | L² | Y² | S² | V² | E² | S³ | N² | | A³ |
| | | B.1.258.17 | <1% | 1.02% | V³ | K² | K¹ | N² | L² | Y² | S² | V² | E² | S³ | N² | | A³ |
| | | B.1.517 | <1% | 0.25% | V³ | K² | N² | N² | L² | Y² | S² | V² | E² | S³ | T | | A³ |
| | WUHAN | WUHAN | — | — | V³ | K² | N² | N² | L² | Y² | S² | V² | E² | S³ | N² | | A³ |
| PCR Amplimer length (bases) | | | | | | (5) 199 | | | | | (6) 151 | | | | | | |
| | | | | | | | | I692V | A701V | T716I | G769V | D796 Y | F888L | S982A | T1027I | D1118H | V1176F |
| | Street | Pango | | | | | | S2 subunit ( AA 686-1273 | | | | | | | | | |
| | name | lineage | Q613H | D614G | H655Y | Q677P/H | P681H | Fusion Peptide (788-806) | | | | | | | | | |
| VOC | UK | B.1.1.7 | Q² | G¹ | H³ | Q² | H¹ | I² | A² | I | G³ | D³ | F³ | A | T³ | H | V³ |
| VOC | Cal | B.1.429 | Q² | G¹ | H³ | Q² | P² | I² | A² | T³ | G³ | D³ | F³ | S³ | T³ | D³ | V³ |
| | L452R | | | | | | | | | | | | | | | | |
| VOC | | B.1.427 | Q² | G¹ | H³ | Q² | P² | I² | A | T³ | G³ | D³ | F³ | S³ | T³ | D³ | V³ |
| VOC | Brazil | P.1 | Q² | G¹ | Y | Q² | P² | I² | A² | T³ | G³ | D³ | F³ | S³ | I | D³ | F |
| VOC | SA | B.1.351 | Q² | G¹ | H³ | Q² | P² | I² | V¹ | T³ | G³ | D³ | F³ | S³ | T³ | D³ | V³ |
| VOI | NYC | B.1.526 | Q² | G¹ | H³ | Q² | P² | I² | A/V¹ | T³ | G³ | D³ | F³ | S³ | T³ | D³ | V³ |
| | (Ho | | | | | | | | | | | | | | | | |
| | et al.) | | | | | | | | | | | | | | | | |
| VOI | NYC | B.1.525 | Q² | G¹ | H³ | H | P² | I² | A² | T³ | G³ | D³ | L | S³ | T³ | D³ | V³ |
| VOI | Rio | P.2 | Q² | G¹ | H³ | Q² | P² | I² | A² | T³ | G³ | D³ | F³ | S³ | T³ | D³ | F |
| | de Jan. | | | | | | | | | | | | | | | | |
| | | B.1.2 | Q² | G¹ | H³ | Q² | P² | I² | A² | T³ | G³ | D³ | F³ | S³ | T³ | D³ | V³ |
| | | B.1, B.1.1, | Q² | G¹ | H³ | Q² | P² | I² | A² | T³ | G³ | D³ | F³ | S³ | T³ | D³ | V³ |
| | | B.1.234 | | | | | | | | | | | | | | | |
| | | B.1.1.519 | Q² | G¹ | H³ | Q² | H¹ | I² | A² | T³ | G³ | D³ | F³ | S³ | T³ | D³ | V³ |
| | | B.1.526.1 | Q² | G¹ | H³ | Q² | P² | I² | A/V¹ | T³ | G³ | D³ | F³ | S³ | T³ | D³ | V³ |
| | | B.1.526.2 | Q² | G¹ | H³ | Q² | P² | I² | A² | T³ | G³ | D³ | F³ | S³ | T³ | D³ | V³ |
| | | B.1.596 | Q² | G¹ | H³ | Q/P¹ | P² | I² | A² | T³ | G³ | D³ | F³ | S³ | T³ | D³ | V³ |
| | | R.1 | Q² | G¹ | H³ | Q² | P² | I² | A² | T³ | V | D³ | F³ | S³ | T³ | D³ | V³ |
| | | B.1.575 | Q² | G¹ | H³ | Q² | H¹ | I² | A² | I | G³ | D³ | F³ | S³ | T³ | D³ | V³ |
| | | B.1.243, | Q² | G¹ | H³ | Q² | H¹ | I² | A² | T³ | G³ | D³ | F³ | S³ | T³ | D³ | V³ |
| | | B.1.1.207 | | | | | | | | | | | | | | | |
| | US | B.1.375 | Q² | G¹ | H³ | Q² | P² | I² | A² | T³ | G³ | D³ | F³ | S³ | T³ | D³ | V³ |
| | B.1.1.1, B.1.416, | | Q² | G¹ | H³ | Q² | P² | I² | A² | T³ | G³ | D³ | F³ | S³ | T³ | D³ | V³ |
| | B.1.1.33, B.1.311, | | | | | | | | | | | | | | | | |
| | B.1.1.122 | | | | | | | | | | | | | | | | |
| | Brazil | B.1.1.28 | Q² | G¹ | H³ | Q² | P² | I² | A² | T³ | G³ | D³ | F³ | S³ | T³ | D³ | F |
| | (ORIG) | | | | | | | | | | | | | | | | |
| | Andrah | B.1.1.420 | Q² | G¹ | H³ | Q² | P² | I² | A² | T³ | G³ | D³ | F³ | S³ | T³ | D³ | V³ |
| | Pradesh | | | | | | | | | | | | | | | | |
| | | A.23.1 | H1 | D2 | H³ | Q² | R¹ | I² | A² | T³ | G³ | D³ | F³ | S³ | T³ | D³ | V³ |
| | | A.27 | H¹ | D² | H³ | Q² | R¹ | I² | A² | T³ | G³ | D³ | F³ | S³ | T³ | D³ | V³ |
| | | A.28 | Q² | D² | Y | Q² | P² | I² | A² | T³ | G³ | Y | F³ | S³ | T³ | D³ | V³ |
| | Mink/ | B.1.1.298 | Q² | D² | Y | Q² | P² | I² | A² | T³ | G³ | D³ | F³ | S³ | T³ | D³ | V³ |
| | Cluster V | | | | | | | | | | | | | | | | |
| | | B.1.1.318 | Q² | G¹ | H³ | Q² | P² | I² | A² | T³ | G³ | D³ | F³ | S³ | T³ | D³ | V³ |
| | | B.1.160 | Q² | G¹ | H³ | Q² | H¹ | I² | A² | T³ | G³ | D³ | F³ | S³ | T³ | D³ | V³ |
| | | B.1.177 | Q² | G¹ | H³ | Q² | P² | I² | A² | T³ | G³ | D³ | F³ | S³ | T³ | D³ | V³ |
| | | B.1.177.80 | Q² | G¹ | H³ | Q² | P² | I² | A² | T³ | G³ | D³ | F³ | S³ | T³ | D³ | V³ |
| | | B.1.258 | Q² | G¹ | H³ | Q² | P² | I² | A² | T³ | G³ | D³ | F³ | S³ | T³ | D³ | V³ |
| | | B.1.258.14 | Q² | G¹ | H³ | Q² | P² | I² | A² | T³ | G³ | D³ | F³ | S³ | T³ | D³ | V³ |
| | | B.1.258.17 | Q² | G¹ | H³ | Q² | P² | I² | A² | T³ | G³ | D³ | F³ | S³ | T³ | D³ | V³ |
| | | B.1.517 | Q/H | G¹ | H³ | Q² | P² | I² | A² | T³ | G³ | D³ | F³ | S³ | T³ | D³ | V³ |
| | WUHAN | WUHAN | Q² | D² | H³ | Q² | P² | I² | A² | T³ | G³ | D³ | F³ | S³ | T³ | D³ | V³ |

| | | |
|---|---|---|
| PCR Amplimer length (bases) | (7) 88 | (8) 135 |

¹AA mutation-hybridizes to mutation specific probe
²AA identical to hCoV-19/Wuhan/WIV04/2019 (WIV04)-official reference sequence employed by GISAID (EPI_ISL_402124) Hybridizes to reference specific probe
³Potential probe target

The detailed design of the (8) TMA (NASBA) primer pairs is displayed in Table 2. Special emphasis is directed to the addition of a T7 promoter sequence (TAATACGACTCACTATAGG) to each forward primer and a detector probe binding nucleotide sequence (SEQ ID NO: 69, GTCAATGAGCAAAGGTATTAGA) that is complementary to the fluorescent detector probe nucleotide sequence (SEQ ID NO: 68, CY3-TTTTCTAATACCTTTGCTCATTGACTTT) to each reverse primer. Core forward and reverse primer sequences common to both RT-PCR and TMA are shown within parenthesis. Spacer sequences TACAAT (SEQ ID NO: 70) and GCATA (SEQ ID NO: 71) flank the T7 promoter sequence in the forward primer.

The detector probe and correspondingly, the detector probe binding nucleotide sequence, may be altered to include a large number of alternative sequences, thus enabling the binding of a variety of detector probes, keeping in mind a Detector Probe length in the range of 15 to 30 bases with a GC content from about 40% to about 60%.

TABLE 2
Primer Sequences used for TMA Reactions

| SEQ ID | Amplimer | | | |
|---|---|---|---|---|
| NO | # | Target | Gene | Primer Sequence (5’ to 3’) |
| 1 | 1 | AA11-33 | Spike | TCTAATACCTTTGCTCATTGAC-(TTTTTT |
| | | | | TCTTGTTTTATTGCCACTAGTC) |
| 2 | | AA11-33 | Spike | TACAAT-TAATACGACTCACTATAGG-GCA |
| | | | | TA-(TTTTTGTCAGGGTAATAAACA |
| | | | | CCACGTG) |
| 3 | 2 | AA64-80 | Spike | TCTAATACCTTTGCTCATTGAC-(ACCTT |
| | | | | TCTTTTCCAATGTTACTTGGTTC) |
| 4 | | AA64-80 | Spike | TACAAT-TAATACGACTCACTATAGG-GCA |
| | | | | TA-(TTTTATGTTAGACTTCTCAGTGGAAG |
| | | | | CA) |
| 5 | 3 | AA126-157 | Spike | TCTAATACCTTTGCTCATTGAC-(TTTCTT |
| | | | | ATTGTTAATAACGCTACTAATG) |
| 6 | | AA126-157 | Spike | TACAAT-TAATACGACTCACTATAGG-GCA |
| | | | | TA-(TTTCATTCGCACTAGAATAAACTCT |
| | | | | GAA) |
| 7 | 4 | AA213-260 | Spike | TCTAATACCTTTGCTCATTGAC-(TTTTA |
| | | | | AGCACACGCCTATTAATTT AGTG) |
| 8 | | AA213-260 | Spike | TACAAT-TAATACGACTCACTATAGG-GCA |
| | | | | TA-(TTTCCACATAATAAGCTGCAGCACCA |
| | | | | GC) |
| 9 | 5 | AA408-456 | Spike | TCTAATACCTTTGCTCATTGAC-(TGTAA |
| | | | | TTAGAGGTGATGAAGTCAGA) |
| 10 | | AA408-456 | Spike | TACAAT-TAATACGACTCACTATAGG-GCA |
| | | | | TA-(TTTAAAGGTTTGAGATTAGACTTC |
| | | | | CTAA) |
| 11 | 6 | AA475-505 | Spike | TCTAATACCTTTGCTCATTGAC-(TTTT |
| | | | | ATTTCAACTGAAATYTATCAGGCC) |
| 12 | | AA475-505 | Spike | TACAAT-TAATACGACTCACTATAGG-GCA |
| | | | | TA-(TTTAAAGTACTACTACTCTGTATG |
| | | | | GTTG) |
| 13 | 7 | AA603-618 | Spike | TCTAATACCTTTGCTCATTGAC-(TTTA |
| | | | | GTGTTATAACACCAGGAACAAATA) |
| 14 | | AA603-618 | Spike | TACAAT-TAATACGACTCACTATAGG- |
| | | | | GCATA-TTTTGCATGAATAGCAACA |
| | | | | (GGGACTTCT) |
| 15 | 8 | AA677-707 | Spike | TCTAATACCTTTGCTCATTGAC-(TTTT |
| | | | | GCAGGTATATGCGCTAGTTATCAG) |
| 16 | | AA677-707 | Spike | TACAAT-TAATACGACTCACTATAGG-GCA |
| | | | | TA-(TTTTGGTATGGCAATAGAGTTATTA |
| | | | | GAG) |

### Microarray Hybridization Probe Sequences

Hybridization probe sequences designed for hybridization to (n) multiplex RT-PCR amplimers, remain suitable without modification for hybridization to the corresponding set of (8) multiplex TMA (NASBA) (RT-T7) amplimers. Table 3 describes wild-type, mutant and universal probes (U) suitable for detecting both wild type and mutant sequences (“U” in Target identifier column, Table 3).

TABLE 3
Hybridization Probe Sequences used for Microarray Analysis

| SEQ ID | Amplimer | | |
|---|---|---|---|
| NO | # | Target | Probe Sequence (5’ to 3’) |
| 17 | 1 | 13U-SE-RE1.2 | TTTTTCTAGTCTCTAKTCAGTGTGTTTTTT |
| 18 | 1 | 13S-SE-RE1.2 | TTTTTTTGTCTCTAGTCAGTGTTTTTTTTT |
| 19 | 1 | 13I-SE-RE1.1 | TTTTTTTAGTCTCTATTCAGTGTTTTTTTT |
| 20 | 1 | 2OU-SE-RE1.1 | TTTTTTAATYTTACAAMCAGAACTCTTTTT |
| 21 | 1 | 20T-SE-RE1.1 | TTTTTTTATCTTACAACCAGAACCTTTTTT |
| 22 | 1 | 20T-SE-RE1.2 | TTTTTTTATCTTACAACCAGAACTTTTTTT |
| 23 | 2 | AA69-70 HV | TTTTTCCCATGCTATACATGTCTCTGTTTT |
| | | | TT |
| 24 | 2 | AA69-70 DEL | TTTTTTTTTCCATGCTATCTCTGGGATTTT |
| | | | TT |
| 25 | 2 | AA_D80A | TTTTTCAGAGGTTTGMTAACCCTGTCTTT |
| | | | TTT |
| 26 | 2 | AA_D80_ | TTTTTTTGGTTTGATAACCCTGCTTTTTTT |
| 27 | 2 | AA_80A | TTTTTTTGGTTTGCTAACCCTGCTTTTTTT |
| 28 | 3 | AA D138Y | TTTTATTTTGTAATKATCCATTTTTGTTTT |
| 29 | 3 | AA D138_ | TTTTTCTTTGTAATGATCCATTTTCTTTTT |
| 30 | 3 | AA_138Y | TTTTTTTTTGTAATTATCCATTTTCTTTTT |
| 31 | 3 | AA W152C | TTTTTAGTTGKATGGAAAGTGAGTTCTTTT |
| 32 | 3 | AA W152_ | TTTCTCTAAAAGTTGGATGGAAACTCTTC |
| | | | T |
| 33 | 3 | AA_152C | TTTCTTCAAAGTTGTATGGAAAGCCTTCT |
| | | | T |
| 34 | 4 | AA A243_ | TTTTTTTTCAAACTTTACTTGCTTTACTCT |
| | | | TT |
| 35 | 4 | AA_243DEL | TTTTTTTTCAAACTTTACATAGAAGCCTTT |
| | | | TT |
| 36 | 4 | AA_R246_ | TTTTCTACATAGAAGTTATTTGACTCCCTT |
| | | | TT |
| 37 | 4 | AA_2461 | TTTTCTGCTTTACATATGACTCCTGGTTTT |
| | | | TT |
| 38 | 4 | AA D253G | TTTCTACTCCTGGTGRTTCTTCTTCATTTT |
| 39 | 4 | AA D253_ | TTTTTTCCCTGGTGATTCTTCTTTCTTTTT |
| 40 | 4 | AA_253G | TTTTTTCCCTGGTGGTTCTTCTTTTTTTTT |
| 41 | 5 | AA 439K+N440K | TTTTTAATTCTAAMAAKCTTGATTCTAATT |
| | | | TT |
| 42 | 5 | AA N439_+N440_ | TTTTTAATTCTAACAATCTTGATTTCTTTT |
| 43 | 5 | AA_N439_+_440K | TTTTTTATTCTAACAAGCTTGATTTTTTTT |
| 44 | 5 | AA_439K+N440_ | TTTTcTATTCTAAAAATCTTGATTTCTTTT |
| 45 | 5 | AA L452R | TTTCTATAATTACCTGTATAGATTGTCTTT |
| 46 | 5 | AA L452_ | TTTTTTTAATTACCTGTATAGATTTCTTTT |
| 47 | 5 | AA_452R | TTTTTCATAATTACTGGTATAGATCTTTTT |
| 48 | 6 | AA S477_ | TTTTTTCGCCGGTAGCACACCTCTTTTTT |
| | | | T |
| 49 | 6 | AA_477N | TTTTCTTCCGGTAACACACCTTTTTTTTTT |
| 50 | 6 | AA V483A+E484K | TTTTTTAATGGTGTTRAAGGTTTTAATTTT |
| | | | TT |
| 51 | 6 | AA V483_+E484_ | TTTTTTCTGGTGTTGAAGGTTTTACTTTTT |
| 52 | 6 | AA V483_+_484K | TTTTTTTATGGTGTTAAAGGTTTTCTTTTT |
| 53 | 6 | AA_483A+E484_ | TTTTTTTATGGTGCTGAAGGTTCTTTTTTT |
| 54 | 6 | AA N501Y | TTTTTTTTCCAACCCACTWATGGTGTTTT |
| | | | TTTT |
| 55 | 6 | AA N501_ | TTTTTTTTACCCACTAATGGTGTCTTTTTT |
| 56 | 6 | AA N_501Y | TTTTTTTTACCCACTTATGGTGTCTTTTTT |
| 57 | 8 | AA Q677P/H | TTTTTTATCAGACTCMGACTAATTCTCTTT |
| | | | TT |
| 58 | 8 | AA Q677_ | TTTTTTCCAGACTCAGACTAATTTCTTTTT |
| 59 | 8 | AA_677P | TTTTTCTTCAGACTCCGACTAATCTTTTTT |
| 60 | 8 | AA_677H1 | TTTTTTCCAGACTCATACTAATTTCTTTTT |
| 61 | 8 | AA_677H2 | TTTTTTCCAGACTCAGACTAATTTCTTTTT |
| 62 | 8 | AA P681H | TTTTTTCAGACTAATTCTCMTCGGCTTTTT |
| 63 | 8 | AA P681_ | TTTTTTTCTAATTCTCCTCGGCGTTTTTTT |
| 64 | 8 | AA_681H | TTTTTTTTTAATTCTCATCGGCGTTTTTTT |
| 65 | 8 | AA A701V | TTTTCACTTGGTGYAGAAAATTCAGTTTTT |
| 66 | 8 | AA A701_ | TCTTCTTCTTGGTGCAGAAAATTATTCTTT |
| 67 | 8 | AA_701V | TCTTCTTCTTGGTGTAGAAAATTATTCTTT |

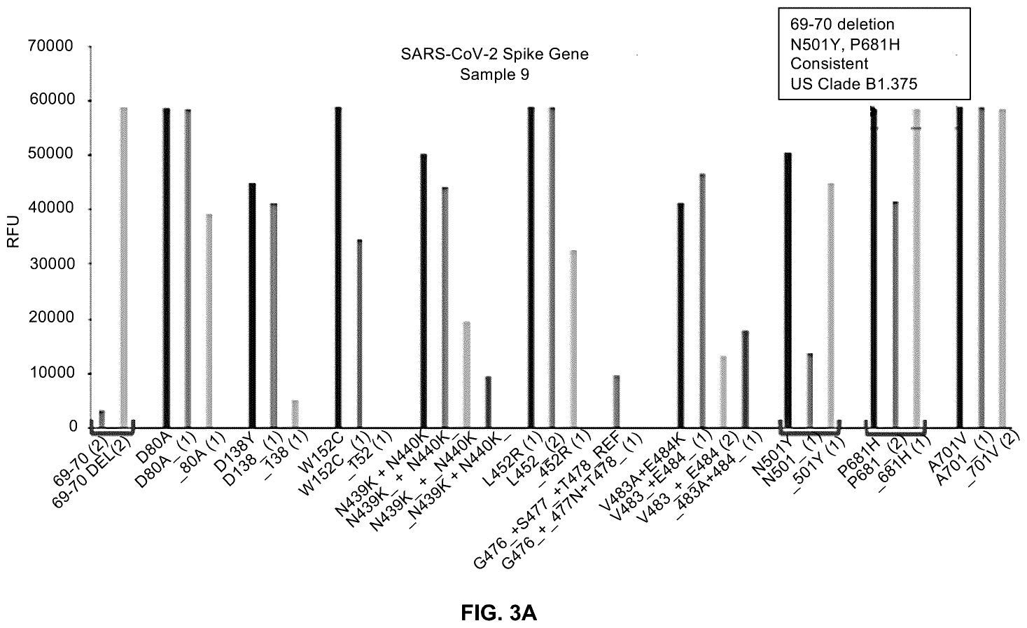

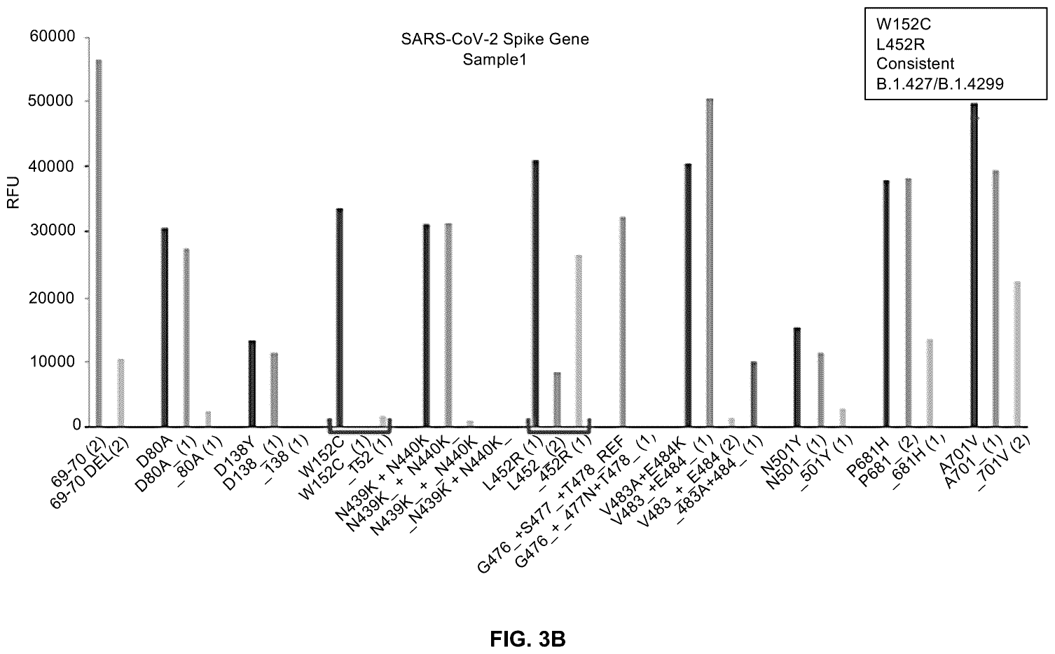

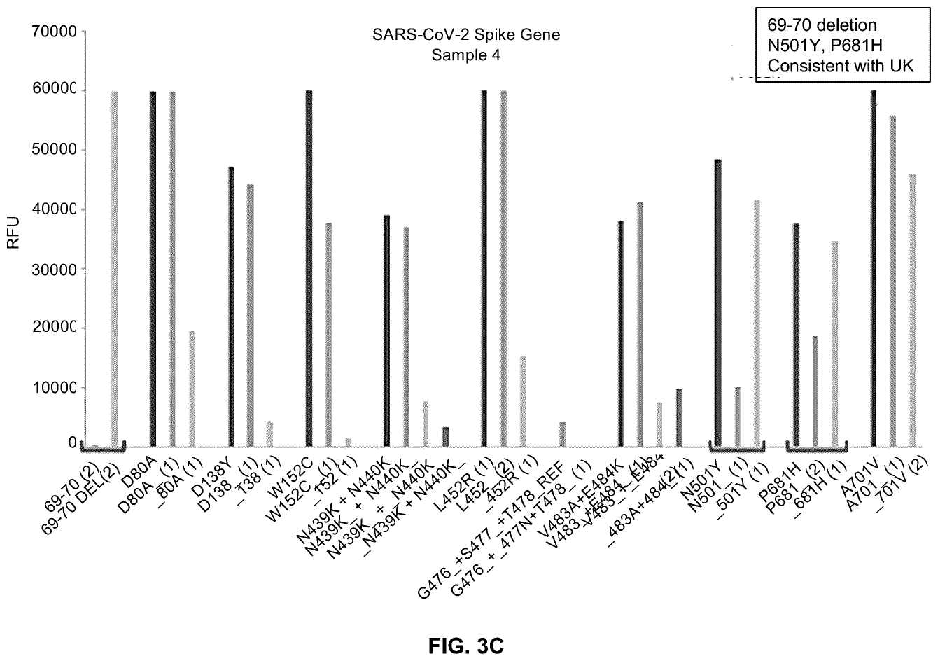

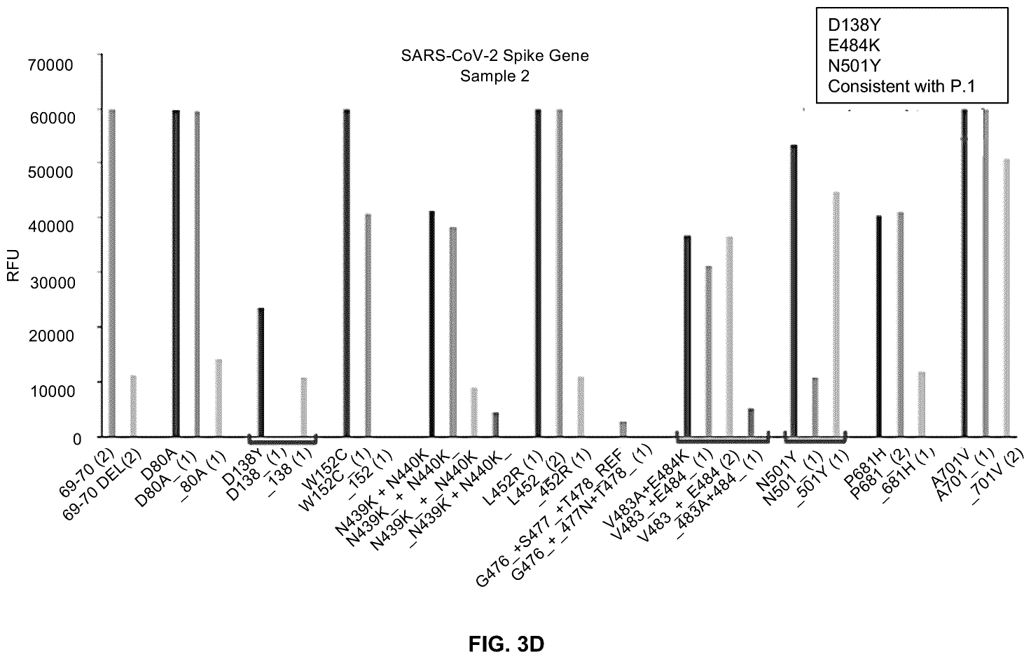

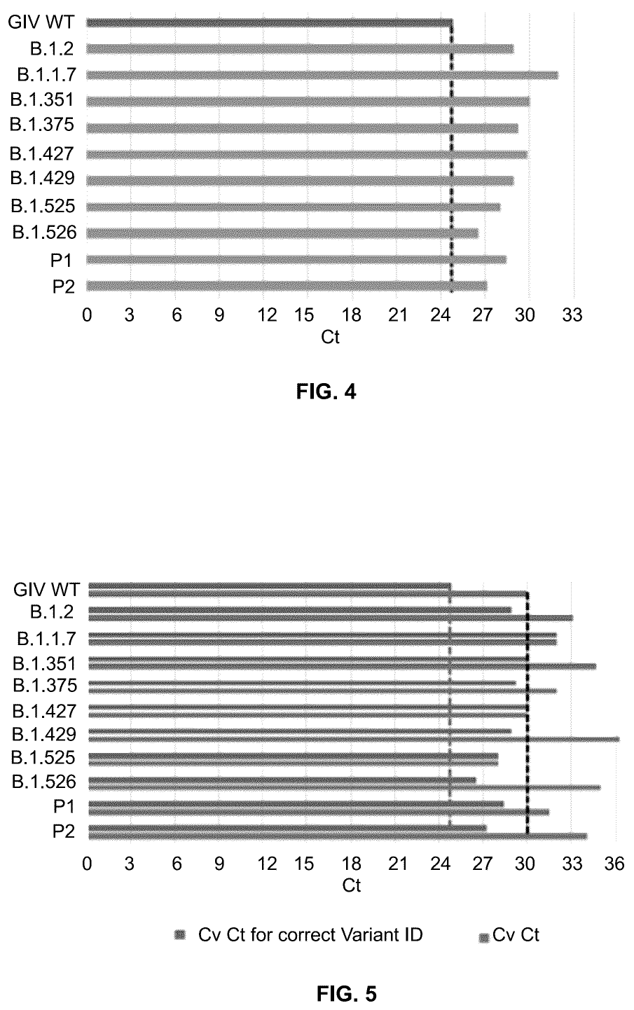

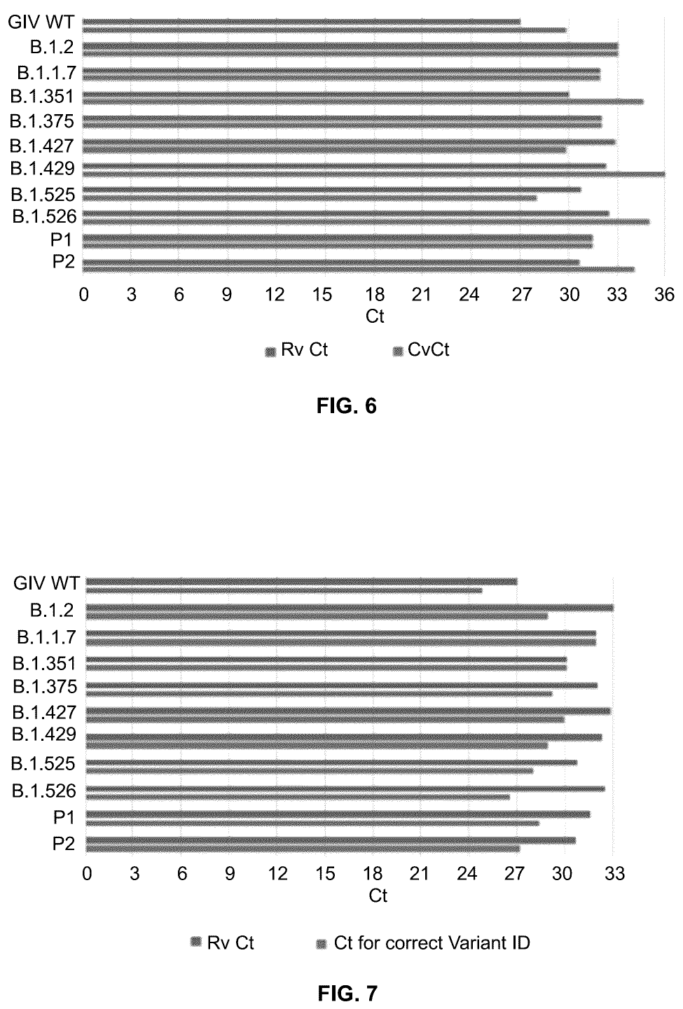

The assay was verified via NIH-RADx Blinded Variant panel (comprising the clinical samples) from Emory RADx Clinical Core site. These clinical samples were confirmed as bonafide variants via Next Generation Sequencing. The blinded panel comprised 10 different variants, 52 samples in serial dilutions resulting in 100% detection of all Variants on the DetectX-Cv assay and signed off by the RADx Variant task Force. The assay performance showed a similar “Limit of Analysis” (LOA) for all variants tested, which in terms of the qPCR analysis performed by RADx on each sample, comprised a LoA of about 28-30, to be compared to a Limit of Detection (LOD) for the same test of 32-35. The Residual unprocessed samples from the NIH-RADx Blinded Variant panel were sent to TriCore Reference Labs, New Mexico. TriCore analysis had also identified 100% of the Variants. The Limit of Analysis obtained in these TriCore data was identical or, in two instances one dilution lower (higher Ct) than that obtained previously. TriCore Reference Labs also processed 28 clinical samples. FIGS. 3A-3D show a subset of the results. Individual plots are available upon request for the full complement of clinical samples. FIGS. 4-7 illustrate the abilities of the arrays to correctly identify variants.

### EXAMPLE 3

### Multiplex NASBA Microarray Detection of a Mixture of SARS-COV-2, Influenza A and Influenza B

### Multiplex NASBA Microarray Assay

Genomic RNA reference reagents and oligonucleotides (NASBA primers, Detector Probes and Hybridization Probes) that are used are listed in Table 4 and Table 5.

TABLE 4
Genomic RNA Reference Reagents

| Reagent | Source | Identifier |
|---|---|---|
| SARS-CoV-2 virus | ATCC | NR-52507 |
| Genomic RNA | | |
| Influenza A virus | ATCC | VR-95DQ |
| (H1N1) Genomic | | |
| RNA | | |
| Influenza B virus | ATCC | VR-1804DQ |
| (Yamagata) | | |
| Genomic RNA | | |
| Universal Human | ThermoFisher | QS0639 |
| Reference RNA | Scientific | |
| NASBA liquid kit | Life Sciences | SKU: NWK-1 |
| | Advanced | |
| | Technologies Inc. | |
| Nuclease-Free | ThermoFisher | AM9938 |
| Water | Scientific | |
| Streptavidin, R- | ThermoFisher | S866 |
| phycoerythrin | Scientific | |
| conjugate (SAPE) | | |

TABLE 5
Sequence of NASBA primers, Detector Probes and Hybridization Probes

| SEQ ID | | |
|---|---|---|
| NO | Primers and Probes | Nucleotide Sequences |
| | SARS-CoV-2 | |
| | NASBA Primers | |
| 72 | 447-FP-99296-Flag1-23 | TTTTAATCGGTGCTCTTGACCAAATTGC |
| | | AATCTTGATTCTAAGGTTGGTG |
| 73 | 501.RP-99243-T7-2 | GAATTTAATACGACTCACTATAGGGATA |
| | | ATAAAGTACTACTACTCTGTATGGTTG |
| | SARS-CoV-2 | |
| | Hybridization Probes | |
| 74 | 452U-SE-RE1.1 | TTTCTATAATTACCDGTATAGATTGCTTT |
| 75 | 452L-SE-RE1.2 | TTTTTTTAATTACCTGTATAGATTTCTTTT |
| 76 | 452R-SE-RE1.5 | TTTTTCATAATTACCGGTATAGATCTTTTT |
| 77 | 452Q-SE-RE1.2 | TTTTTTTTAATTACCAGTATAGACTTTTTT |
| 78 | S477N-SE-RE1.2 | TTTTTCGCCGGTARCAMACCTTGTATTTTT |
| 79 | T478K-SE-RE1.1 | TTTTTCGGTAGCAMACCTTGTAATGTTTTT |
| 80 | T478-SE-RE1.2 | TTTTTTTGGTAGCACACCTTGTTTTTTTTT |
| 81 | 478K-SE-RE1.5 | TTTTTTTGTAGCAAACCTTGTATTTTTTTT |
| 82 | 484U-SE-RE1.1 | TAATGGTGYTRMAGGTTTTAATTTTTT |
| 83 | 483V484E-SE-RE1.7 | TTTTTTCTGGTGTTGAAGGTTTTACTTTTT |
| 84 | 484K-SE-RE1.5 | TTTTTTTATGGTGTTAAAGGTTTTCTTTTT |
| 85 | 483A-SE-RE1.3 | TTTTTTTATGGTGCTGAAGGTTCTTTTTTT |
| 86 | V483_._484A-RE1.2 | TTTTTTCGGTGTTGCAGGTTTTATCTTTTT |
| 87 | F490S-SE-RE1.1 | TTTTCTAATTGTTACTYTCCTTTACAATTTTT |
| 88 | F490-SE-RE1.1 | TTTTTTTTTGTTACTTTCCTTTACTTTTTT |
| 89 | 490S-SE-RE1.1 | TTTTTTTTTGTTACTCTCCTTTACTTTTTT |
| 90 | 493.494-SE-RE1.3 | TCCTTTACAAYCATATGGTTTTTTTT |
| 91 | | TTACGATCATATAGTTTCCTTTTT |
| 92 | S494-SE-RE1.1 | CTTTACAATCATATGGTCTTTTT |
| 93 | 494P-SE-RE1.1 | TTTTTCTCTTTACAACCATATGGTCTTTTT |
| 94 | _493R._496S-RE1.1 | TTTTTCTTACGATCATATAGTTTCTTTTTT |
| 95 | 501UNI-SE-RE1.1 | TTTTTTTTCCAACCCACTWATGGTGTTTTTTT |
| | | T |
| 96 | 501N-SE-RE1.4 | TTTTTTTTACCCACTAATGGTGTCTTTTTT |
| 97 | 501Y-SE-RE1.4 | TTTTTTTTACCCACTTATGGTGTCTTTTTT |
| | SARS-CoV-2 | |
| | Detector Probes | |
| 98 | 99089-Biotin(Biotin- | /5Biosg/TTTTAATCGGTGCTCTTGACC |
| | Flag1) | AAATTG |
| 99 | Biotinylated-Amp6-tag | /5Biosg/TTTTTTGAGAGAGATATTTCA |
| | (501.RP-99243-T7-2) | ACTGATTT |
| 100 | Cy3-Flag3-tag | /5Cy3/TTTCTACCGTACTCTAGCTTT |
| | (Paer3-RP-1.4-C3) | |
| 101 | Cy3-TMA5-tag | /5Cy3/TTTCTGTTGAGTTATCCCTTT |
| | (TMA5-15MER-C3) | |
| | Influenza A | |
| | NASBA Primers | |
| 102 | infA-FP-Lau-Flag3-23 | TTTCTACCGTACTCTAGCTACTT |
| | | CTAACCGAGGTCGAAACGTA |
| 103 | infA-RP-99083-T7- | GAATTTAATACGACTCACTATAG |
| | TMA5-23 | GGATAACTCAACAGGCATTYTGGACAAAKCG |
| | | TCTACG |
| | Influenza A | |
| | Hybridization Probe | |
| 104 | lnfA.7.univ-pubRev | TTTTTCGTGCCCAGTGAGCGAGGACTGCATT |
| | | TTT |
| | Influenza B | |
| | NASBA Primers | |
| 105 | infB-FP-99086-Flag3-23 | TTTCTACCGTACTCTAGCTATCCTCAAC |
| | | TCACTCTTCGAGCG |
| 106 | infB-RP-99089-T7- | GAATTTAATACGACTCACTATAGGGAT |
| | TMA5-23 | AACTCAACAGATCGGTGCTCTTGACCA |
| | | AATTGG |
| | Influenza B | |
| | Hybridization Probe | |
| 107 | lnfB.8.univ-pub | TTTTCCAATTCGAGCAGCTGAAACTGC |
| | | GGTGTTTTT |
| | Influenza A and B | |
| | Detector Probes | |
| 108 | Flag3-Biot | /5Biosg/TTTCTACCGTACTCTAGCTTT |
| 109 | TMA5-15MER-BIOT | /5Biosg/TTTCTGTTGAGTTATCCCTTT |
| | B2M | |
| | NASBA Primers | |
| 110 | B2M-FP-99320-Flag3 | TTTCTACCGTACTCTAGCTGCCTGCCGTG |
| | | TGAACCATGTGA |
| 111 | B2M-RP-99150-T7- | AATTTAATACGACTCACTATAGGGATAAC |
| | TMA5 | TCAACAGTGGAATTCATCCAATCCAAATG |
| | | CG |
| | B2M | |
| | Hybridization Probe | |
| 112 | B2M_RE2.1 | TTTTTAGCATCATGGAGGTTTGAAGTTTTT |
| | B2M | |
| | NASBA Detector | |
| | Probe | |
| 108 | Flag3-Biot | /5Biosg/TTTcTACCGTACTCTAGCTTT |
| 109 | TMA5-15MER-BIOT | /5Biosg/TTTCTGTTGAGTTATCCCTTT |

During the multiplex TMA (NASBA) reaction, primer mediated isothermal amplification produces RN amplicons which acquire detector probe binding sequences which are suitable to bind fluorescently labeled detector probes that are introduced in the hybridization or wash buffers, thereby forming a [Hybridization Probe-RNA amplicon-Detector Probe] “Sandwich”. The Detector Probes are DNA oligonucleotides synthesized with a biotin group at their 5′ termini (Table 5). When mixed with Streptavidin modified Phycoerythrin (SAPE), a SAPE-detector probe complex is formed, thereby labeling the RNA amplicon as bound to its cognate hybridization probe.

### Method

### Step 1: NASBA Reaction

Primer mix and reaction components are listed in Table 6 and Table 7. The reaction is carried out according to the manufacturer's manual (Life Sciences Advanced Technologies) with modifications. Human beta-2-microglobulin RNA is used as a control. Briefly, the method is as follows.
1. Denaturation of RNA template and anneal primers to the template. For each reaction,
   1) Mix 6.7 ul of 3× NASBA reaction buffer (NECB-1-24) and 3.3 ul of 6× Nucleotide Mix (NECN-1-24) and warm the mixture at 41° C. for 5 minutes.
   2) To the mixture, add 1 ul of RNA template, and 3.38 ul primer mix (see Table 6 for primer mix composition).
   3) Mix gently and incubate the mixture at 95° C. for 5 minutes, followed by a 10-min incubation at 41° C.
2. Amplification of targets.
   1) Warm the enzyme mixture (NEC-1-24) to 41° C.
   2) Add 5 ul of the enzyme mixture to the reaction and incubate at 41° C. for 1 hour, followed by a 10-min at 65° C. before hybridization.

TABLE 6
Primer mix for one reaction

| | | Stock | Final |
|---|---|---|---|
| | | concen- | concentration |
| Primer | Volume | tration | in the reaction |
| B2M-FP-99320-Flag3 | 0.25 ul | 2 uM | 25 nM |
| B2M-RP-99150-T7-TMA5 | 0.25 ul | 2 uM | 25 nM |
| infA-FP-Lau-Flag3-23 | 0.75 ul | 2 uM | 75 nM |
| infA-RP-99083-T7-TMA5-23 | 0.75 ul | 2 uM | 75 nM |
| infB-FP-99086-Flag3-23 | 0.5 ul | 2 uM | 50 nM |
| infB-RP-99089-T7-TMA5-23 | 0.5 ul | 2 uM | 50 nM |
| 447-FP-99296-Flag1-23 | 0.188 ul | 2 uM | 18.8 nM |
| 501.RP-99243-T7-2 | 0.188 ul | 2 uM | 18.8 nM |
| Total volume | 3.38 ul | | |

TABLE 7
NASBA reaction mixtures

| Component | Volume |
|---|---|
| 3 × Buffer (NECB-24) | 6.7 ul |
| 6 × Nucleotide (NECN-24) | 3.3 ul |
| Primer mix | 3.38 ul |
| Nuclease-Free water | 0.62 ul |
| Sample | 1 ul |
| Enzyme mix (NEC-1-24) | 5 ul |
| Total volume | 20 ul |

### Step 2: Hybridization of NASBA Products to the Microarray at Room Temperature

1. Prepare the 96-well microarray plate for TMA (NASBA) hybridization. A microarray is printed at the bottom of each well. 1) Rinse each reaction well with 200 ul of molecular grade water once, followed by a 5-min 200 □I water staying in the well. 2) After aspiration of water, dispense 200 □I of pre-hybridization solution (Table 8) into the well and leave there for 5 minutes.
2. Prepare the 96-well microarray plate for TMA (NASBA) hybridization. A microarray is printed at the bottom of each well. 1) Rinse each reaction well with 200 □I of molecular grade water once, followed by a 5-min 200 □I water staying in the well. 2) After aspiration of water, dispense 200 □I of pre-hybridization solution (Table 9) into the well and leave there for 5 minutes.
3. Aspirate pre-hybridization solution, dispense 80 □I of the NASBA hybridization solution in the well, and kept in dark for 60 minutes.
4. Wash of microarray with washing buffer (0.15×SSC). After the hybridization, aspirate the hybridization solution and wash wells four times. One time rinse followed by a 10-min wash (keep washing buffer in the well for 10-min), then 2× rinse. Dry the plate by spinning the plate face down in a Micro Array Plate Centrifuge for 3-5 minutes.

TABLE 8
Pre-hybridization Buffer

| | Volumes needed |
|---|---|
| | for 8 wells to be |
| 96-Well Plate | pre-hybridized |
| Molecular biology grade water | 1.397 mL |
| Pathogen Dx DetectX | 0.415 mL |
| Buffer 1 (20 × SSC) | |
| PathogenDx DetectX | 0.218 mL |
| Buffer 2 (50 × Denhardt's) | |

TABLE 9
PDx NASBA microarray hybridization mixture

| | Volume | Stock concentration |
|---|---|---|
| Product of | 20 ul | |
| NASBA reaction | | |
| 20 × SSC | 7 ul | |
| 50 × Denhardt's | 7 ul | |
| solution | | |
| Biotin-Flagl | 0.64 ul | 50 uM |
| Biotin-Flag3 | 1.28 ul | 50 uM |
| Biotin-TMA5 | 0.64 ul | 50 uM |
| Biotin-Amp6 | 0.64 ul | 50 uM |
| SAPE | 42.8 ul | 2.5 uM |
| Total volume | 80 ul | |

### Step 3: Scanning the Microarray and Data Acquisition

1. Scan the hybridized microarray plate in a desktop Sensovation scanner using the “PathogenDx™ Assay 002” program.
2. Submit the “Scan Results” file folder to the “Image Folder” in the PDx Dropbox.
3. The folder automatically begins to upload, and the Augury™ Software analyzes the data and directly deposits the reports into the “Reports” folder within Dropbox.
4. The relative fluorescent units (RFU) of hybridized signals corresponding to different probes on the microarray can be obtained with the “RearrangeDotScore” software analyzing the PAX file in the “Reports” folder.

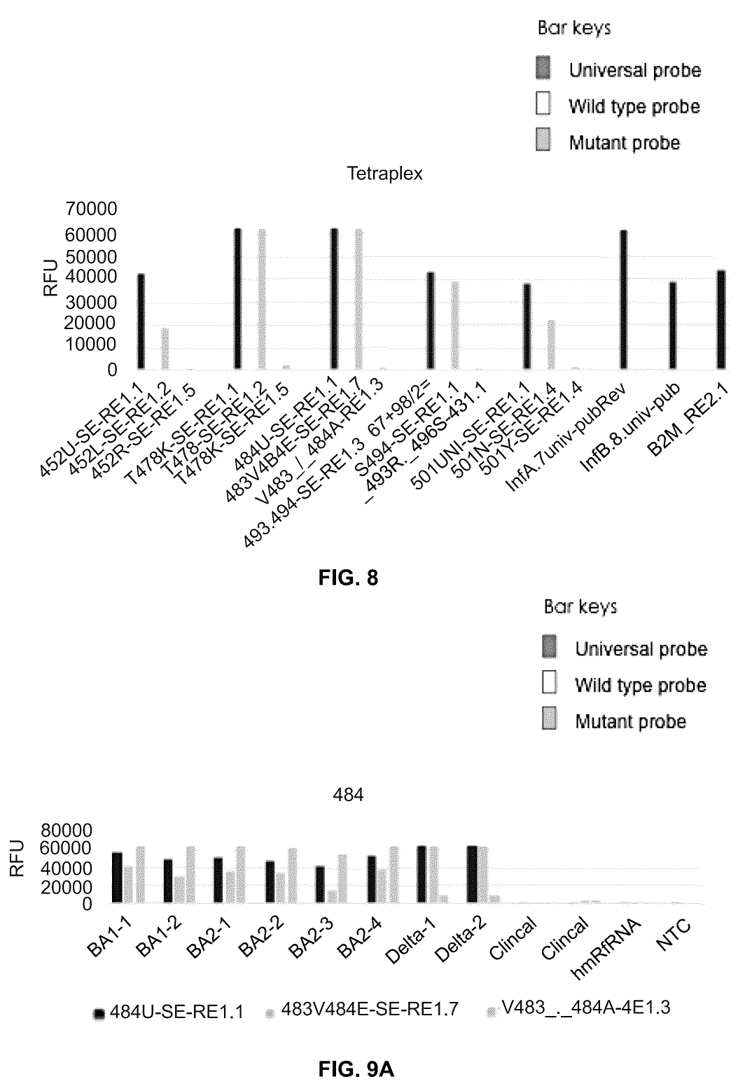

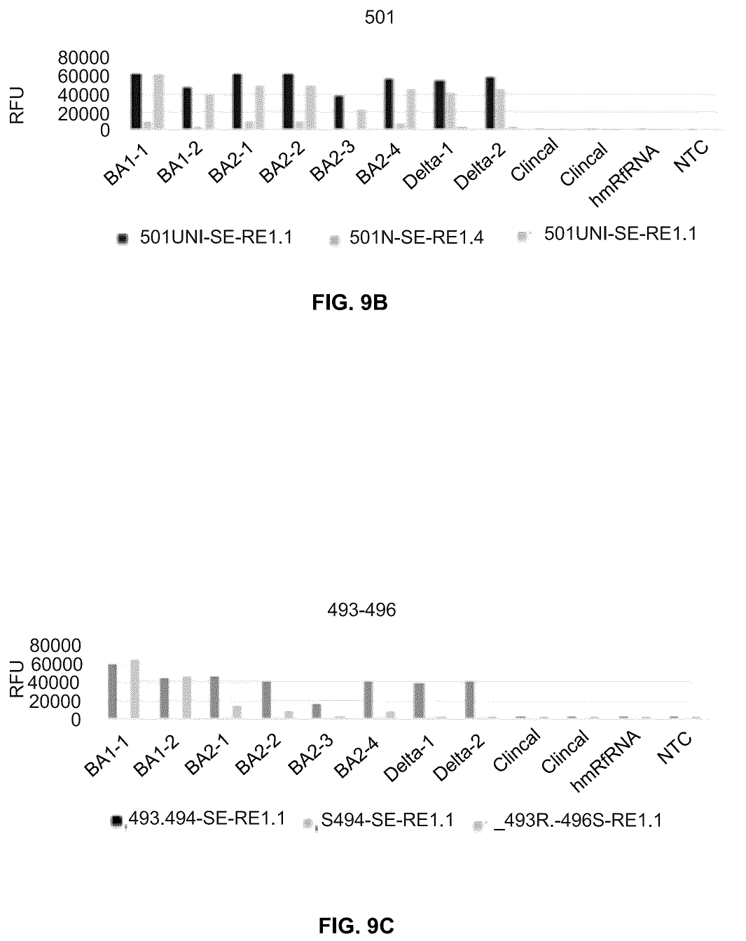

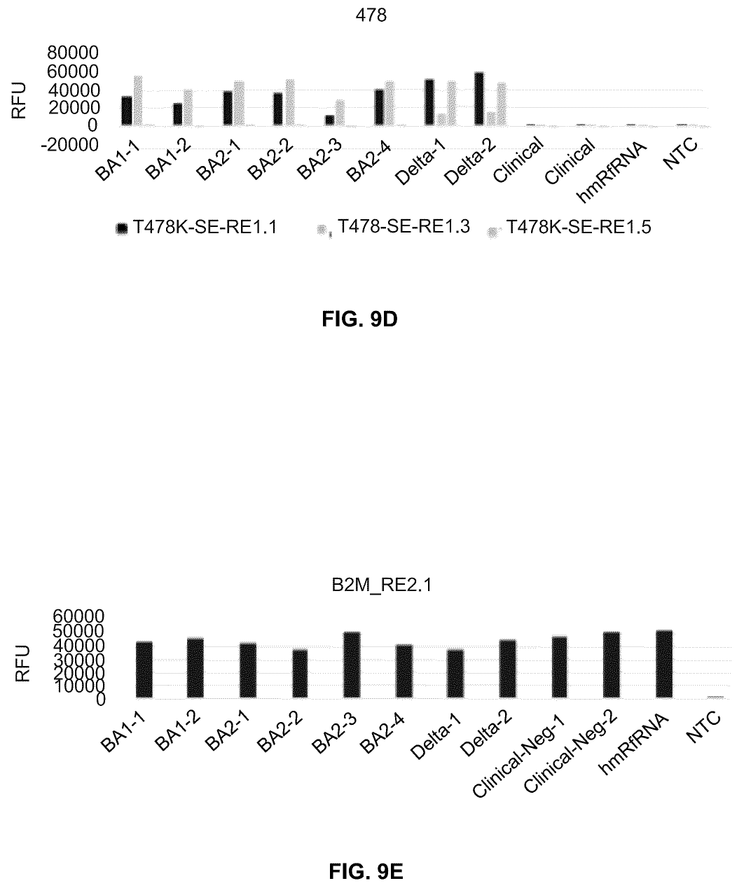

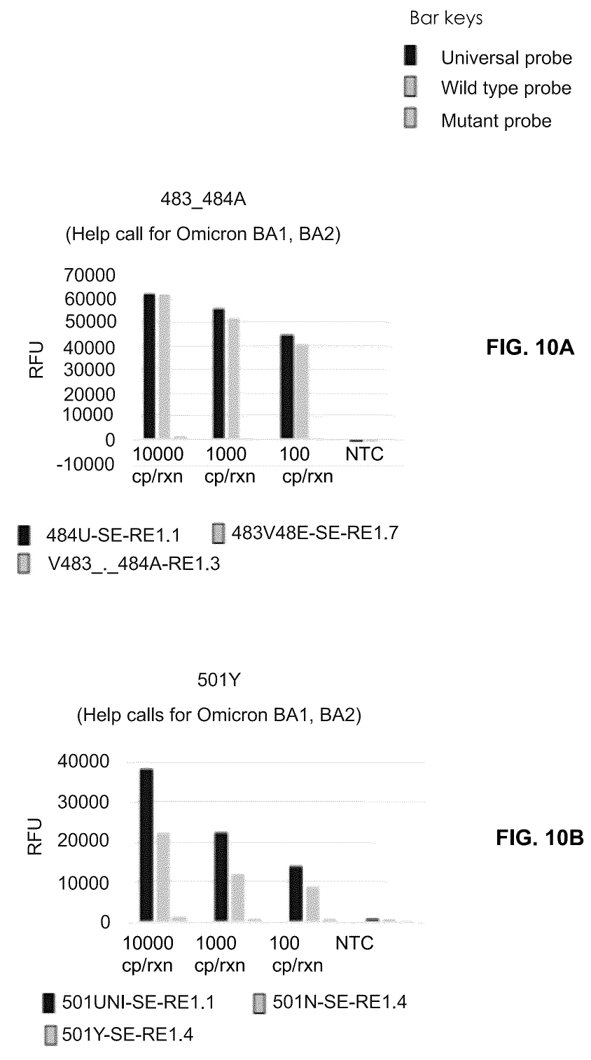

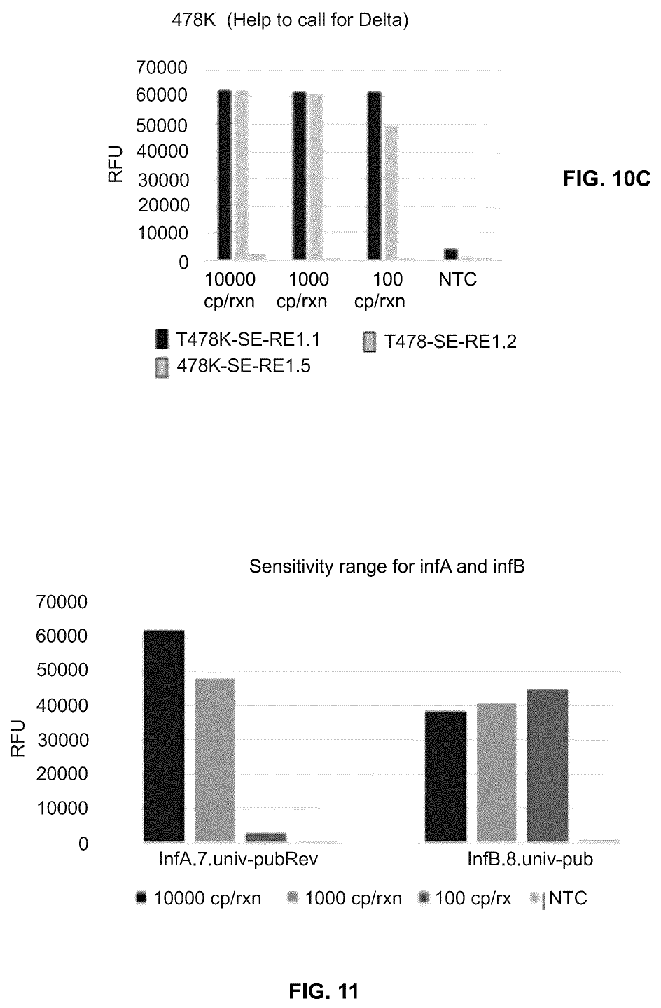

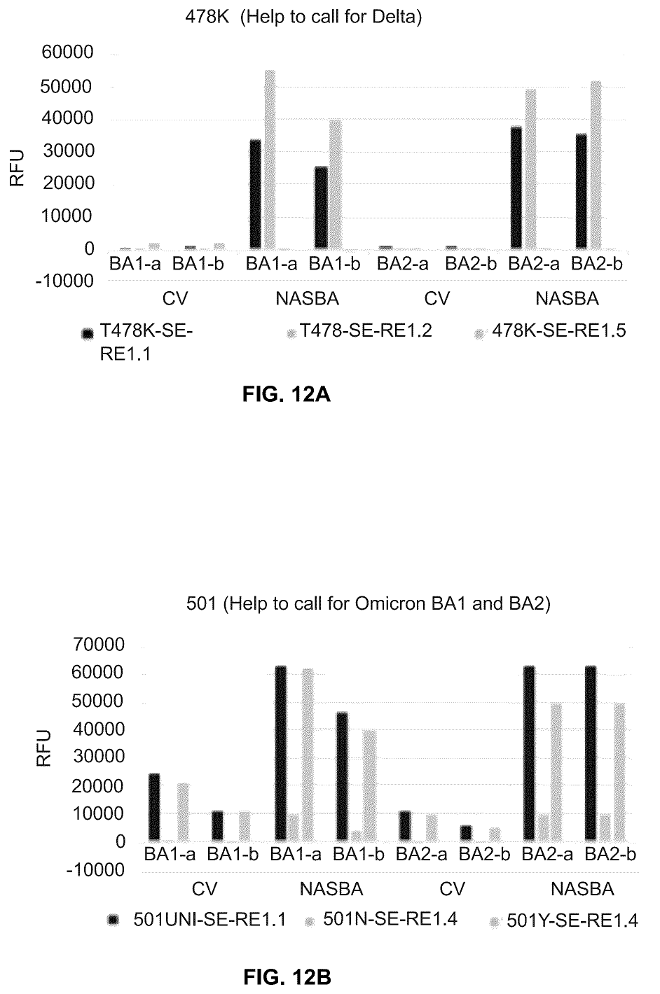

The multiplex TMA (NASBA) technology, coupled to microarray detection detects and resolves a mixture of viral genomes, i.e., SARS-COV-2, influenza A, and influenza B RNA, and also purified human RNA (B2M gene) as a single multiplex reaction (FIG. 8). The multiplex NASBA-Microarray technology also distinguishes SARS-COV-2 Variants including Omicron BA1, BA2, and Delta (FIGS. 9A-9E) based on the unique pattern of single nucleotide polymorphism presented within the region of the Spike gene that has been amplified in the present multiplex NASBA reaction. This observation confirms that the present method is suitable to yield RNA amplicons which may be successfully analyzed by microarray hybridization, under hybridization conditions where sequences can be resolved based on a single nucleotide change. When coupled to microarray analysis, these data reveal an analytical LOD for the multiplex NASBA reaction of 100 genome copies/reaction for SARS-COV-2 and influenza B, and 1000 genome copies/reaction influenza A (FIGS. 10A-10C and FIG. 11). Direct comparison of the present multiplex NASBA reaction with a multiplex RT-PCR reaction which has been designed to amplify the same Spike gene region (FIGS. 12A-12B) shows that the multiplex NASBA reaction of the present invention can be as sensitive as a multiplex RTPCR predicate in detecting SARS-COV-2 Omicron variant BA1 in clinical samples.

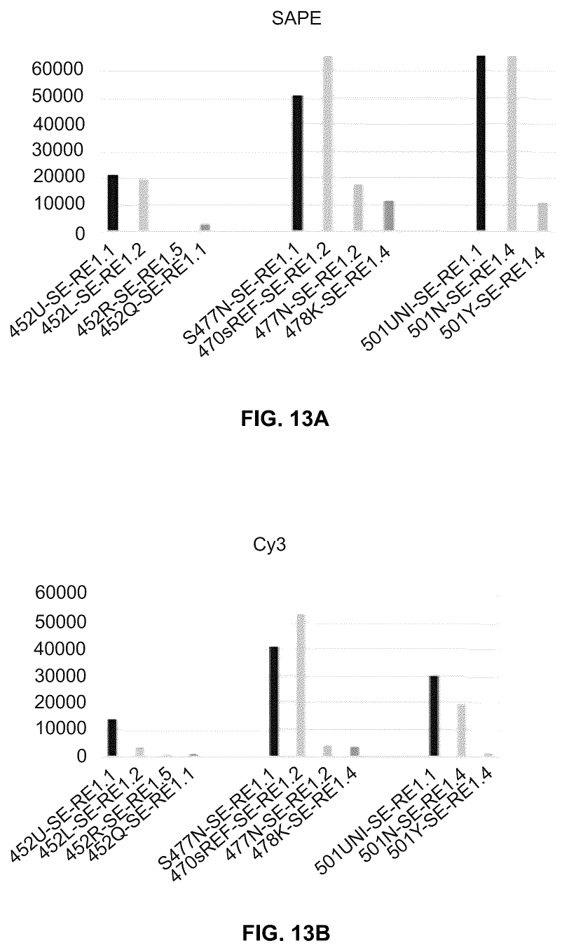

Also shown is that a biotinylated detector probe may be used which, upon binding to SAPE (Streptavidin, R-Phycoerythrin conjugate) multiplex NASBA amplicon binding to microarray probes, may be detected via the resulting fluorescent SAPE fluorescence signals, with sensitivity similar to that obtained with identical detector probes labeled covalently at time of their synthesis with CY3 dye. The data (FIGS. 13A-13B) shows that such non-covalent SAPE labeling is more sensitive than direct covalent labeling of the detector probe with an organic fluor such as CY3. The data also suggest that other types of detection methods, such as other organic fluors or phosphors or ceramic Quantum Dots, may be similarly used to detect hybridization of multiplex NASBA amplicons via the creation of a streptavidin conjugate and its (analogous) binding to biotin modification on the detector probe.

### EXAMPLE 4

### Multiplex NASBA Microarray Detection of Ribosomal RNA Hypervariable Regions in Prokaryotes or Eukaryotes

It is well known in the art that in bacteria and more generally prokaryotes and in fungi and more generally eukaryotes that the ribosomal RNA (rRNA) 16s and 23S in prokaryotes and 18S and 28S in eukaryotes each contain hypervariable regions which during the course of evolution have developed patterns of sequence variation within them which can be used to distinguish prokaryotic and eukaryotic genera and, in many cases, to resolve species within those prokaryotic and eukaryotic genera. It is also well known in the art that those hypervariable regions are flanked in most cases by highly conserved sequences, i.e., sequences which do not vary greatly among bacteria and eukaryotes. As a result, it is also well known that universal PCR primers and universal” isothermal amplification primers can be designed so that a single universal primer pair may be used to amplify, by PCR or by isothermal amplification, each of the known hypervariable regions in prokaryotes and in eukaryotes so that the sequence of the resulting amplicons may then be analyzed to determine the identity of the cells or tissues or organisms in the sample under analysis. It is demonstrated that a well-known bacterial hypervariable region (HV3) for which universal PCR primer pairs are well known is similarly amplified by use of a corresponding multiplex TMA (NASBA) primer pair, designed as provided herein, thus enabling multiplex TMA (NASBA) amplification of one or more such hypervariable ribosomal RNA sites as a single multiplex isothermal amplification reaction.

Table 10 identifies an optimized PCR primer pair sequence (16SFP, 16SRP) and the corresponding multiplex NASBA primer pair sequence (16SFP-Flag3, 16SRP-T7+TMAS-23). The main distinction between the PCR vs the corresponding multiplex NASBA primer pair is the introduction of a flanking sequence in one NASBA primer which upon reverse transcription and the action of RNAse H generates a T7 promoter and a first detector probe binding site (16SRP-T7+TMA5-23) at one end of the resulting RT+RNase H double stranded DNA product. In turn, the other member of the NASBA primer pair creates a single detector probe binding site at the opposite end of the resulting double stranded DNA RT+RNase H product (16SFP-Flag3).

TABLE 10
Representative multiplex TMA (NASBA)
primers, detector probes and
hybridization probe for rRNA Analysis

| SEQ | | |
|---|---|---|
| ID NO | Sequence Name | Sequences |
| | PCR primer | |
| 113 | 16SFP | CACACTGGRACTGAGACACG |
| 114 | 16SRP | GTATTACCGCGGCTGCTGGCA |
| | NASBA primer | |
| 115 | 16SFP-Flag3 | TTTCTACCGTACTCTAGCTA- |
| | | CACACTGGRACTGAGACACG |
| 116 | 16SRP-T7+ | GAATTTAATACGACTCACTAT |
| | TMA5-23 | AGGGATAACTCAACAG-GTAT |
| | | TACCGCGGCTGCTGGCA |
| | Detector Probe | |
| 108 | Flag3-Biot 100 | /5Biosg/TTTCTACCGTACT |
| | | CTAGCTTT |
| 109 | TMA5-15MER- | /5Biosg/TTTCTGTTGAGTT |
| | BIOT | ATCCCTTT |
| | Listeria spp. | |
| | hybridization | |
| | probe | |
| | for PCR & | |
| | NASBA amplicons | |

As described herein, upon the action of T7 polymerase, multiple RNA amplicons are produced from such a multiplex TMA (NASBA) primer pair, resulting from the combined action of [RT+RNaseH+T7]. Moreover, those RNA amplicon molecules are then ready to hybridize to a cognate nucleic acid probe, which, upon ordinary Watson-Crick pairing, can be used to identify the genera or the species associated with the rRNA template of interest in the original sample. One such representative hybridization probe is described (Listeria spp. hybridization probe) which upon hybridization to such a multiplex NASBA amplicon specifically identifies the sample as containing rRNA from one of the several known Listeria species, that is, the probe identifies the sample as “Listeria spp”.

As described herein, several TMA (NASBA) primer pairs like those in Table 10may be deployed in parallel to amplify a larger set of rRNA hypervariable sites as a single multiplex TMA (NASBA) reaction. The amplified RNA product of each element of such a multiplex TMA (NASBA) reaction in turn is then hybridized in parallel to a single microarray containing many hybridization probes, like those in Table 10. In that way, a combination of multiplex TMA (NASBA) amplification at multiple hypervariable sites in bacteria and in eukaryotes produce multiple RNA amplicons in parallel which then bind by sequence specific hybridization to a very large number of hybridization probes arrayed on a surface of a microarray. The resulting pattern of microarray hybridization, as visualized by the binding of fluorescently tagged detector probes to the detector probe binding sites created at the end of the RNA amplicons may then be used to identify which bacteria and fungi are present in a sample, based on the measured pattern of binding to the microarray. Samples of interest suitable for analysis are a surface swab, an air sample, a food sample, a blood sample or a urine sample or any other biological or environmental sample for which an understanding of its microbial content would be valuable.

### EXAMPLE 5

Multiplex Isothermal NASBA Amplification and Microarray Detection of Antibiotic Resistance Determinants in ESKAPE+Pathogens

### Isothermal, Multiplex NASBA Chemistry

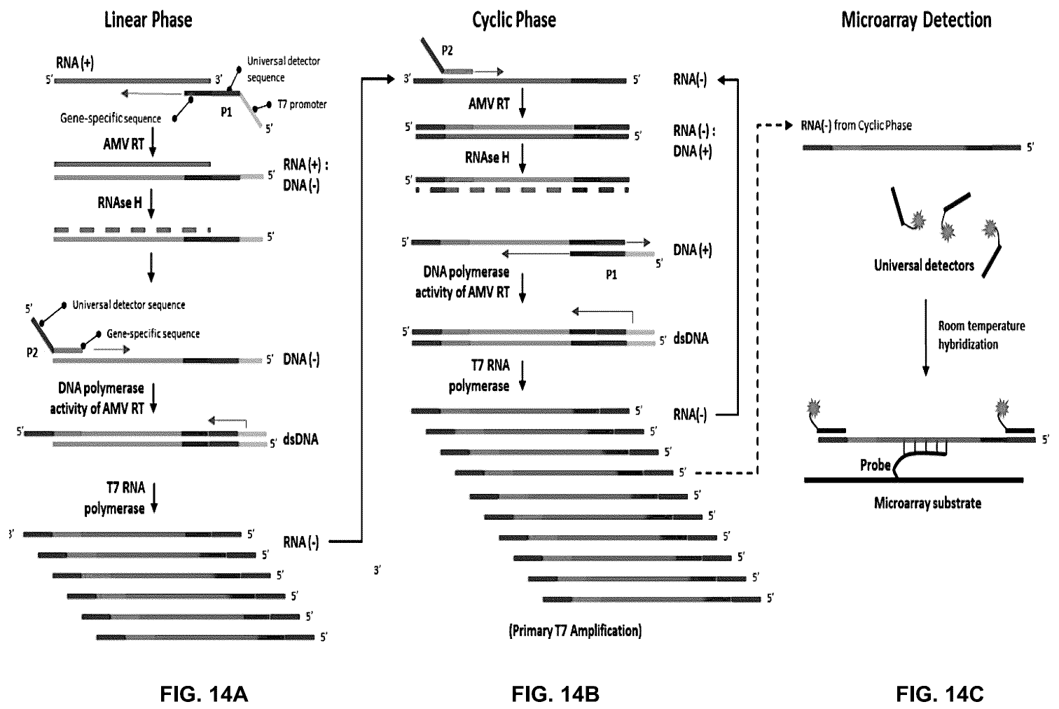

The method is predicated on NASBA isothermal chemistry that targets multi-copy RNA instead of DNA. The standard NASBA reaction has two “stages”. In the first stage (FIG. 14A), AMV reverse transcriptase converts RNA to DNA, and the T7 RNA polymerase generates antisense RNA transcripts (RNA(−)). These transcripts feed into the cyclic phase of the reaction (FIG. 14B), which likewise generates antisense RNA transcripts and dsDNA that contain a T7 promoter. Each newly synthesized dsDNA in the linear or cyclic phases of the reaction can create up to 100 copies of antisense RNA, so that 20 NASBA “cycles” can amplify any target at least at 106-fold. More importantly, NASBA primers multiplex a reverse transcriptase reaction, and target amplification occurs from the T7 promotor, not the primers. NASBA is therefore inherently less prone to primer dimers and non-specific amplification than those chemistries where the amplification step is driven by the primers themselves. Multiplexed detection occurs by hybridizing the amplified, ssRNA transcripts to the PDx array and in the presence of a universal, labeled detection probe (FIG. 14C). Each amplified RNA contains two detector probe binding sites, one on each end. The all-in-one sandwich hybridization reaction occurs at room temperature.

Detection of Microbial rRNA and mRNA in Less Than 4 Hours (n=4)

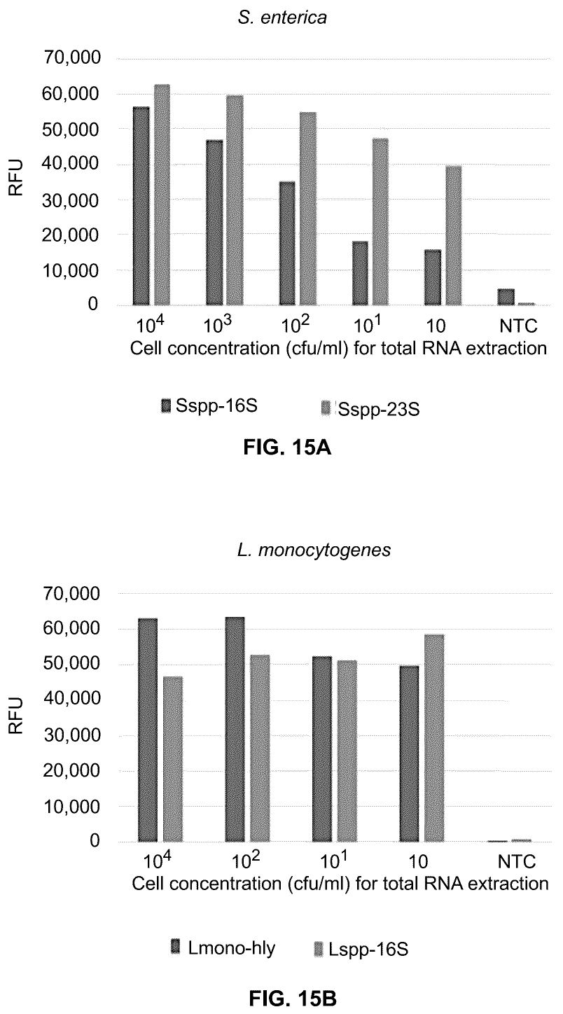

16S and 23S rRNA and the hly gene specific for Listeria monocytogenes were targeted. Total RNA was extracted from isolates with a Zymo Quick RNA/DNA MagBead kit. 5 μL total RNA was added to a 20 μL NASBA reaction and amplified at 41° C. for 2 hrs. After NASBA, the entire contents were mixed with an SSC-hybridization buffer containing a universal detector oligonucleotide, and incubated for 1 hr at room temperature. Washed arrays were imaged on a Sensovation imager, and processed through PDx's fully automated Augury software. An LoD of about 1 CFU/mL (FIGS. 15A-15B) was shown even for the hly mRNA (FIG. 15A).

### Primers and Probes

Primers and probes (Tables 12 and 13) were verified by in silico inclusivity and exclusivity testing against the latest NCBI nucleotide, genome, and gene databases, and conventional PCR amplification/detection specificity. Probes and primers for drug resistance markers were derived from published assays (3-11). All microarray probes were designed for room temperature hybridization and wash conditions. Primers and probes are purchased from IDT, and are verified by mass spectrometry before use.

TABLE 12
Amplification primers
Isothermal Primers

| | | Reverse (5′ → 3′) |
|---|---|---|
| | Forward (5′ → 3′) | Lower case = T7 promoter sequence |
| | Italics = Detector oligo | Bold = spacer sequence |
| | binding site | Italics = Detector oligo binding site |
| 16S rRNA | TTTCTACCGTACTCTAGCT·C | AATT·taatacgactcactataggg·ATAA· |
| HV3 | ACATTGGRACTGAGACACGG | CTCAACAG·GTATTACCGCGGCTG |
| Region | (SEQ ID NO: 117) | CTGGCA |
| | | (SEQ ID NO: 118) |
| 16S rRNA | TTTCTACCGTACTCTAGCT | AATT taatacgactcactataggg ATAA |
| HV6 | CGCACAAGCGGTGGAGCATG | CTCAACAG·ACGAGCTGACGACAG |
| region | T | CCATGCA |
| | (SEQ ID NO: 119) | (SEQ ID NO: 120) |
| imp-1 | TTTCTACCGTACTCTAGCT | AATT taatacgactcactataggg ATAA |
| | GAATAGRRTGGCTTAAYTCTC | CTCAACAG |
| | (SEQ ID NO: 121) | CCAAACYACTASGTTATC |
| | | (SEQ ID NO: 122) |
| kpc | TTTCTACCGTACTCTAGCT | AATT taatacgactcactataggg ATAA |
| | CGGCCGCCGTGCAATACAGT | CTCAACAG |
| | G | GCCGCCCAACTCCTTCAGCAA |
| | (SEQ ID NO: 123) | (SEQ ID NO: 124) |
| oxa-48 | TTTCTACCGTACTCTAGCT | AATT taatacgactcactataggg |
| | CTTAAACGGGCGAACCAAGC | ATAA· CTCAACAG |
| | (SEQ ID NO: 125) | GTTCATCCTTAACCACGCCC |
| | | (SEQ ID NO: 126) |
| ndm-1 | TTTCTACCGTACTCTAGCT | AATT taatacgactcactataggg ATAA |
| | TGACCAGACCGCCCAGATCC | CTCAACAG |
| | TC | CCGCCAGCGCGACCGGCAGGT |
| | (SEQ ID NO: 127) | (SEQ ID NO: 128) |
| vim | TTTCTACCGTACTCTAGCT | AATT taatacgactcactataggg ATAA |
| | AATGGTCTCATTGTCCGTGAT | CTCAACAG |
| | G | TCGCACCCCACGCTGTA |
| | (SEQ ID NO: 129) | (SEQ ID NO: 130) |
| mcr-1 | TTTCTACCGTACTCTAGCT | AATT taatacgactcactataggg ATAA |
| | TAGCTGAACATACACGGCAC | CTCAACAG |
| | AG | CCAAAGACGCGGTACAAGCAAC |
| | (SEQ ID NO: 131) | (SEQ ID NO: 132) |
| ctx-m | TTTCTACCGTACTCTAGCT | AATT taatacgactcactataggg ATAA |
| | ACCAACGATATCGCGGTGAT | CTCAACAG |
| | (SEQ ID NO: 133) | ACATCGCGACGGCTTTCT |
| | | (SEQ ID NO: 134) |
| mecA | TTTCTACCGTACTCTAGCT | AATT taatacgactcactataggg ATAA |
| | TAAACCACCCAATTTGTCTGC | CTCAACAG |
| | C | ATGGTATGCAACAAGTCGTAAA |
| | (SEQ ID NO: 135) | (SEQ ID NO: 136) |
| mecC | TTTCTACCGTACTCTAGCT | AATT taatacgactcactataggg ATAA |
| | GAAAAAAAGGCTTAGAACGC | CTCAACAG· |
| | CTC | GAAGATCTTTTCCGTTTTCAGC |
| | (SEQ ID NO: 137) | (SEQ ID NO: 138) |
| vanA | TTTCTACCGTACTCTAGCT | AATT taatacgactcactataggg ATAA |
| | AGCCGGAAAAAGGCTCTGAA | CTCAACAG· |
| | (SEQ ID NO: 139) | TTTTGCCGTTTCCTGTATCC |
| | | (SEQ ID NO: 140) |
| vanB | TTTCTACCGTACTCTAGCT | AATT taatacgactcactataggg ATAA |
| | GATTTGATTGTCGGCGAAGT | CTCAACAG·TCCTGATGGATGCGG |
| | G | AAGA |
| | (SEQ ID NO: 141) | (SEQ ID NO: 142) |
| Human | TTTCTACCGTACTCTAGCT | AATT taatacgactcactataggg ATAA |
| B2M | GCCTGCCGTGTGAACCATGT | CTCAACAG·TGGAATTCATCCAATC |
| | GA | CAAATGCG |
| | (SEQ ID NO: 143) | (SEQ ID NO: 144) |

TABLE 13
Hybridization probes
Microarray Probes (5′ → 3′)

| Bacterium | HV3a probe | HV3c probe | HV3e probe |
|---|---|---|---|
| Enterococcus | CAAGGATGAGAG | TAACTGTTCATCCC | CGGTATCTAACCA |
| faecium | TAAC | TTG | GA |
| | SEQ ID NO: 145 | SEQ ID NO: 157 | SEQ ID NO: 166 |
| Staphylococcus | GAAGAACATATG | TGTGCACATCTTGA | ACGGTACCTAATC |
| aureus | TGTAAG | C | AG |
| | SEQ ID NO: 146 | SEQ ID NO: 158 | SEQ ID NO: 167 |
| Klebsiella | AGGCGATGAGGT | ATAACCTCATCGAT | ACGTTACCCGCAGA |
| pneumoniae 1 | TAAT | TGAC | SEQ ID NO: 168 |
| | SEQ ID NO: 147 | SEQ ID NO: 159 | |
| Kp2 | AGGCGATAAGGT | TAACCTTGTCGATT | |
| | TAAT | GA | |
| | SEQ ID NO: 148 | SEQ ID NO: 160 | |
| Kp3 | GAAGGCGTTAAG | TAACCTTGGCGATT | |
| | GTTA | GA | |
| | SEQ ID NO: 149 | SEQ ID NO: 161 | |
| Kp4 | AGGCGATAAGGT | TAACCTTGTCGATT | |
| | TAATn | GA | |
| | SEQ ID NO: 150 | SEQ ID NO: 160 | |
| Kp5 | GAAGGCGTTAAG | GAAGGCGTTAAGG | |
| | GTTA | TTA | |
| | SEQ ID NO: 149 | SEQ ID NO: 149 | |
| Kp6 | AAGGCGGTGAGGTT | ATAACCTCATCGAT | |
| | SEQ ID NO: 151 | TGAC | |
| Kp7 | GAAGGCGTTAAG | SEQ ID NO: 159 | |
| | GTTA | | |
| | SEQ ID NO: 149 | | |
| Acinetobacter | GCTACTTTAGTTA | ATACCTAGAGATA | ACGTTACTCGCAG |
| baumannii | ATAC | GTG | A |
| | SEQ ID NO: 152 | SEQ ID NO: 162 | SEQ ID NO:169 |
| Pseudomonas | AAGGGCAGTAAG | ATACCTTGCTGTTT | CGTTACCAACAGA |
| aeruginosa | TTAA | GA | ATA |
| | SEQ ID NO: 153 | SEQ ID NO: 163 | SEQ ID NO: 170 |
| Enterobacter | AAGGTGTTGTGG | CGCAGCAATTGAC | ACGTTACCCGCAGA |
| spp (cloacae) | TTA | GTT | SEQ ID NO: 168 |
| | SEQ ID NO:154 | SEQ ID NO: 164 | |
| (asburiae) | AAGGCGATAAGG | TAACCTTGTCGATT | |
| (hormaechei) | TTAA | GA | |
| | SEQ ID NO: 155 | SEQ ID NO: 160 | |
| Escherichia coli | AAGGGAGTAAAG | TACCTTTGCTCATT | |
| | TTAAT | GA | |
| | SEQ ID NO: 156 | SEQ ID NO: 165 | |
| K.(E.) | GAAGGCGTTAAG | TAACCTTGGCGATT | ACGTTACTCGCAG |
| aerogenes | GTTA | GA | A |
| | SEQ ID NO: 149 | SEQ ID NO: 161 | SEQ ID NO: 169 |
| Bacterium | HV6a probe | HV6c probe | HV6g probe |
| Enterococcus | CCTTACCAGGTC | AGAGCTTCCCCTT | GGGCAAAGTGACA |
| faecium | TTGA | CG | G |
| | SEQ ID NO: 171 | SEQ ID NO: 177 | SEQ ID NO: 186 |
| Staphylococcus | CTTACCAAATCTT | ATCCTTTGACAACT | GGACAAAGTGACA |
| aureus | GAC | CTA | G |
| | SEQ ID NO: 172 | SEQ ID NO: 178 | SEQ ID NO: 187 |
| Klebsiella | TACCTGGTCTTG | CCACAGAACTTTCC | GGAACTGTGAGACA |
| pneumoniae 1 | ACA | SEQ ID NO: 179 | SEQ ID NO: 188 |
| | SEQ ID NO: 173 | | |
| Kp2 | | TCCACAGAACTTAGC | |
| | | SEQ ID NO: 180 | |
| Kp3 | | CCACAGAACTTTC | |
| | | C | |
| Kp4 | | SEQ ID NO: 179 | |
| Kp5 | | TCCACAGAACTTA | |
| | | GC | |
| | | SEQ ID NO: 180 | |
| Kp6 | | CCACAGAACTTTCC | |
| Kp7 | | SEQ ID NO: 179 | |
| Acinetobacter | ACCTGGCCTTGA | ATACTAGAAACTTT | GAATCTAGATACA |
| baumannii | CA | CCA | GGT |
| | SEQ ID NO: 174 | SEQ ID NO: 181 | SEQ ID NO: 189 |
| Pseudomonas | | TGCTGAGAACTTTC | GAACAGAGACACA |
| aeruginosa | | C | G |
| | | SEQ ID NO: 182 | SEQ ID NO: 190 |
| Enterobacter | TACCTGGTCTTG | CCACAGAACTTTC | GGAACTGTGAGAC |
| spp (cloacae) | ACA | C | A |
| | SEQ ID NO: 175 | SEQ ID NO: 179 | SEQ ID NO: 188 |
| (asburiae) | CCTTACCTACTCT | TCCAGAGAACTTTC | GGAACTCTGAGACA |
| | TG | C | SEQ ID NO: 191 |
| | SEQ ID NO: 176 | SEQ ID NO: 183 | |
| (hormaechei) | | CCAGAGAACTTAGC | |
| | | SEQ ID NO: 184 | |
| Escherichia coli | TACCTGGTCTTG | CCACGGAAGTTTT | GGGAACCGTGAGA |
| | ACA | CA | SEQ ID NO: 192 |
| | SEQ ID NO: 173 | SEQ ID NO: 185 | |
| K.(E.) | CCTTACCTACTCT | CCAGAGAACTTAG | GGAACTCTGAGAC |
| aerogenes | TG | C | A |
| | SEQ ID NO: 176 | SEQ ID NO: 184 | SEQ ID NO: 191 |

Controls

| | |
|---|---|
| Negative control | TACCTATGCTGATTCA SEQ ID NO: 193 |
| Pan-HV3 control | CCTACGGGAGGCAGC SEQ ID NO: 194 |

| Resistance | | Resistance | |
|---|---|---|---|
| Gene | Probe | Gene | Probe |
| imp-1 | CTACCCTGGCCCAGGT | Ctx-m | AGATCGTGCGCCGCTGA |
| | CACACT | | TTC |
| | SEQ ID NO: 195 | | SEQ ID NO: 201 |
| kpc | TAACGCCGCCGCCAAT | mecA | CATTTTGAGTTCTGCAGT |
| | TTGT | | ACCG |
| | SEQ ID NO: 196 | | SEQ ID NO: 202 |
| oxa-48 | CCAATAGCTTGATCGC | mecC | AAACCTTTAGACACATTA |
| | CCTCGA | | TTGG |
| | SEQ ID NO: 197 | | SEQ ID NO: 203 |
| ndm-1 | ACTGGATCAAGCAGGA | vanA | CAGTTATAACCGTTCCC |
| | GATC | | GCAGA |
| | SEQ ID NO: 198 | | SEQ ID NO: 204 |
| vim | TGATGAGTTGCTTTTG | vanB | TCAAATCCGGCTGAGCC |
| | ATTG | | ACGGT |
| | SEQ ID NO: 199 | | SEQ ID NO: 205 |
| mcr-1 | AGGCGTGGCTTACGCA | Human B2M | AGCATCATGGAGGTTTG |
| | TATCAG | | AAG |
| | SEQ ID NO: 200 | | SEQ ID NO: 206 |

### Microarray Structure for Automated Detection

The microarray has unique features that enable specific hybridization/detection within less than 3 hours. Specifically, microarray probes are attached to the substrate via a linker that creates a “three-dimensional” structure for each spot. The linker contains a Cy5 (or equivalent) fluorescent reporter for automated test analysis/reporting. The substrate, linker and probe covalently self-assemble within the spot after printing, and are covalently linked to the substrate by a brief UV illumination step. The 3D architecture of each spot accelerates reaction kinetics relative to what can be achieved with two-dimensional arrays. For example, 100 □L of hybridization buffer per array is usually used, which creates a fluid column of ˜1 mm over the array surface. In an unstirred fluid, bulk diffusion is about 1 mm/hr, so the array is not limited by bulk diffusion rates. High sensitivity and specificity is achieved with a 1 hr, room temperature hybridization, at an LoD of about 1 CFU/mL (12,13). TruTips (Akonn Biosystemsi, Frederick, MD) are used to actively mix the hybridization buffer, thus reducing hybridization time to ˜30 min. This is in stark contrast to most other microarray products that require overnight hybridization to achieve clinical performance requirements.

Extraction of Total RNA from Whole Blood

Freshly collected human blood samples were obtained from Innovative Blood Resources (St. Paul, MN). Within 24 hours of collection and overnight shipping, 1 mL of whole blood was spiked with 10,000 cells of each ESKAPE+ pathogen. Cells spiked into PBS served as a control. Total nucleic acid was then extracted from ˜280 μL of contrived blood, plasma, and PBS samples (˜103 bacteria cells) on the OCTA system (Pathogen Dx, Inc. Scottsdale, AZ), using the default, automated blood DNA extraction protocol without modification (14; Akonni Biosystems). In a second iteration, 1 mL of blood, plasma, or PBS was first treated with 0.1 mL of 10% TritonX-100 for 30 seconds, the sample was centrifuged to concentrate intact bacterial cells into a pellet 85, resuspended the pellet was resuspended in 280 μL of PBS, and then proceeded through the automated OCTA sample preparation protocol. In both cases, purified nucleic acids were eluted into 75 μL of elution buffer, and 5 μL used for multiplexed, isothermal amplification (˜65 cell equivalents of total nucleic acid per reaction, assuming 100% sample preparation efficiency). The isothermal multiplex was heated to 65° C. for 5 min, cooled to 41° C., and then 5 μL of NASBA enzyme mixture added to the denatured nucleic acids. After a 2 hr incubation at 41° C., 50 μL of concentrated hybridization buffer (containing universal detector probes) was added to the amplified product, and hybridized the products were hybridized to the microarray for 2 hrs at room temperature. Species-specific probes (Table 13) target the 16S hypervariable 3 and 6 regions. Arrays were washed and imaged on a Sensovation imager (without modification), and processed through the automated Augury software.

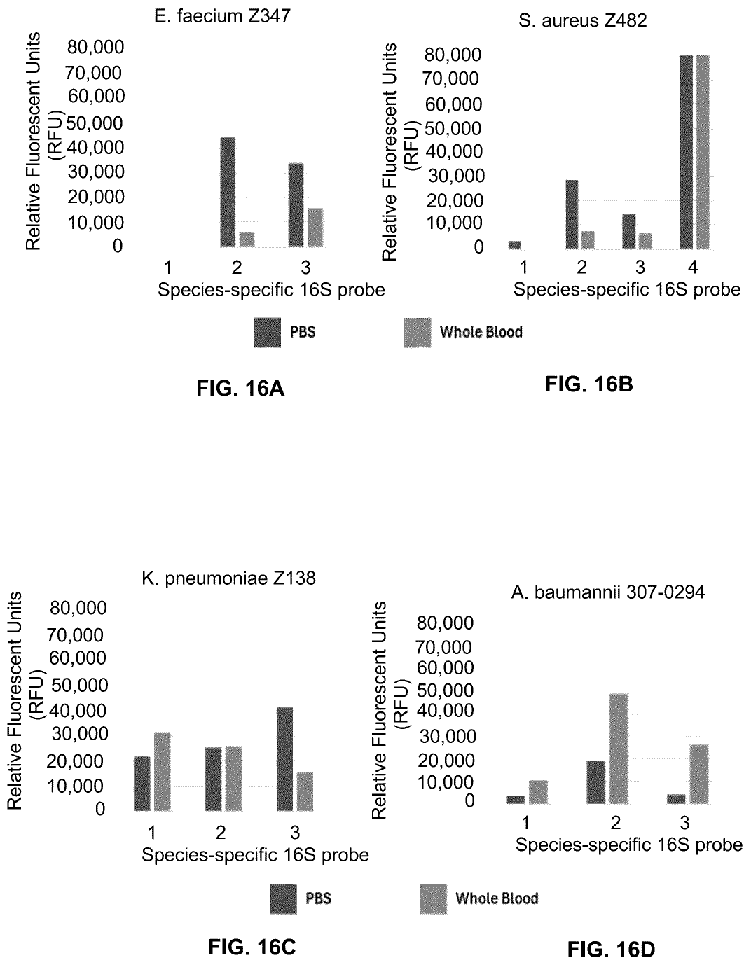

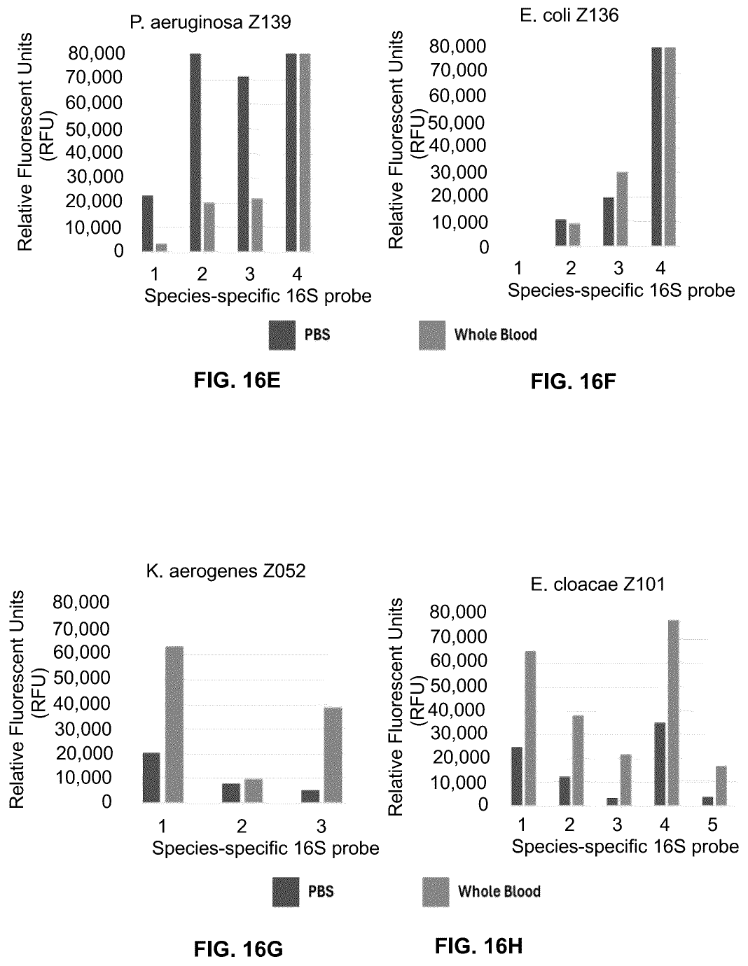

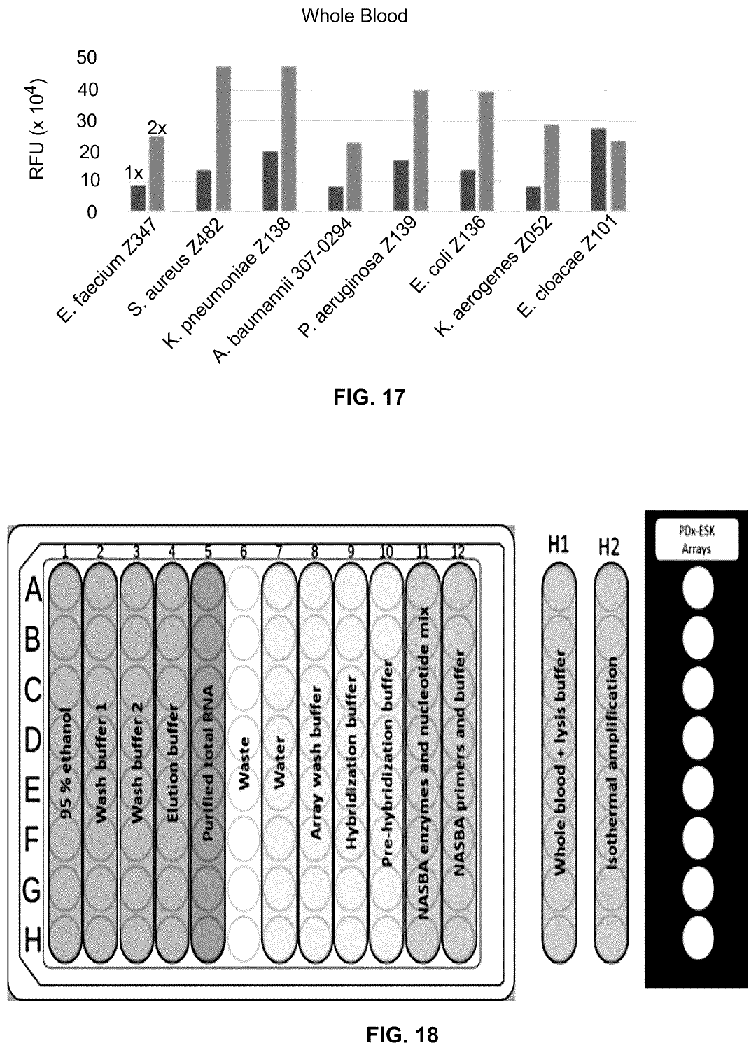

FIGS. 16A-16H demonstrate that 1) total RNA can be automatically extracted from whole blood using a non-optimized TruTip protocol; 2) RNA can be amplified with a (non-optimized) multiplexed, isothermal NASBA reaction; and 3) similar end-point signals can be obtained with ˜65 cell equivalents of total RNA extracted from PBS or whole blood. Doubling the detector probe concentration relative to the standard UTI assay conditions effectively doubles (and sometimes triples) microarray signal intensity for RNA automatically extracted from whole blood (FIG. 18) and PBS (not shown).

### EXAMPLE 6

### Automated, Integrated OctaPlus Platform

The automated, integrated OctaPlus platform is based on the Octa system (PathogenDx, Scottsdale, AZ) which utilizes TruTip technology (Akonni Biosystems, Frederick, MD) to which on-board heating elements are added to enable automated isothermal target amplification. The OctaPlus system platform has a fully automated sample preparation front end that processes primary clinical samples, and 3D microarrays (Example 5) with imaging on the back end to identify bacterial species and their drug resistance determinants.

### Isothermal Master Mix

The total RNA content of a bacterium can range from 1.5 to 200 fg per cell. It is assumed that the “average” RNA content per cell is 100 fg, that each mRNA exists at >10 copies per cell and that each rRNA exists at >1,000 copies per cell. The smallest nucleic acid elution volume from the TruTip is ˜50 μL, and the Octa can reproducibly pipette at least 20 μL between wells. Therefore 20 μL is taken as the starting volume of purified RNA entering the isothermal multiplex, with a total NASBA reaction volume of 30 μL. AMV reverse transcriptase and T7 RNA polymerase (Life Sciences Advanced Technologies) are used. Each primer is at ≤0.1 nM final concentration. A 10 min, 65° C. primer annealing step starts the amplification, and then incubate the NASBA reaction is incuat ˜41° C. for up to 1 hr. The NASBA reaction is terminated by adding the entire contents of the reaction to 50 μL of concentrated (SSC/Denhardt's) hybridization buffer containing 0.5-1 μM universal detector probe. Amplified RNA transcripts are then detected by microarray hybridization. The isothermal master mix enables microarray-based detection of all RNA amplicons across all replicates at ≤0.1-1 pg total RNA per reaction (about 1-10 cells).

### Hybridization Specificity for Amplified Targets

Eight parallel arrays are printed on a borosilicate glass substrate, with each array circumscribed by a frame seal gasket. 50 μL of concentrated hybridization buffer is added directly to the amplification products from above, and hybridize the entire reaction volume is hybridized to the microarray for 30 min at room temperature. The all-in one mixture and hybridization reaction contains 1 μM universal, fluorescently labeled detector, and each amplified, single-stranded RNA transcript contains two detector probe binding sites, one on each end of the target amplicon (FIGS. 14B-14C). After hybridization, microarrays are washed four times with ˜200 μL of low-salt wash buffer, and imaged on a two-color Sensovation imager. One color (e.g., red) is used to identify the spots, and the second color (e.g., green) detects hybridized targets. Resulting.tiff images are automatically processed by the Augury software (PathogenDx), and analyzed by the data analysis software.

Hybridization specificity is achieved with positive detection by the cognate probe(s) across all replicates, no detectable signal (at a signal-to-noise ratio (SNR) >3) on non-target microarray probes and a limit of detection ≤0.1-1 pg total RNA per reaction (≤1-10 cells per reaction). While unexpected, a non-specific microarray signal can be minimized by decreasing the salt concentration in the wash buffer to increase wash stringency. The probe-specific SNR thresholds may be adjusted for declaring a probe “Detected” vs. “Not Detected”. Probe sequences may be modified, or primers/probes for 23S rRNA and/or internally transcribed spacer (ITS) regions may be included. Nucleic acid analogues or minor groove binders may be introduced into the probes to enhance specificity.

### ESKAPE+ Pathogen RNA Extraction From Whole Blood Optimization

FIGS. 16A-16H illustrate the recovery of RNA from whole blood using the described automated protocol (14), followed by multiplexed NASBA/microarray detection with ˜65 cell equivalents of RNA entering the multiplexed NASBA reaction. Total RNA sample preparation efficacy for ESKAPE+ pathogens spiked into whole blood may be optimized, and at pathogen loads expected during infection. Leftover, de-identified, pathogen-free, whole blood is purchased (Innovative Blood, Minneapolis, MN). The same 12 microbial strains from above are spiked into whole blood to achieve final concentrations of 1,000, 300, 100, 10, and 1 CFU/mL. Non-spiked whole blood is the negative control.

The optimization test starts with 1 mL of whole blood where spiked samples are treated with 0.1% Triton X-100 for 30 sec, and intact bacterial cells are pelleted by centrifugation at 4,000×g (15). This procedure lyses human red and white blood cells, releases human nucleic acids to the supernatant, and leaves bacteria intact and concentrated in the resulting pellet. The procedure may be scaled up to an entire 8 mL blood EDTA tube, if needed. Because human nucleic acids end up in the supernatant, the TruTip does not become “saturated” with human DNA or preclude microbial RNA from binding the matrix. The bacterial pellet is resuspended in 0.2 mL of PBS, load samples are loaded onto the OctaPlus, and automatically extract total nucleic acids are extracted according to TruTip protocol for whole blood (14). Then 20 μL of total RNA eluant is introduced into the multiplex amplification reaction and perform microarray hybridization and washing are performed as described above.

Optimized RNA extraction from whole blood enables correct microarray detection of each target microorganism and drug resistance marker across all 5 replicates with no false positive signals on non-target probes, and an estimated clinical LoD (cLoD) between 1-10 CFU/mL whole blood. The cLoD depends on automated sample preparation efficacy. It is noted that the current automated blood protocol does recover amplifiable RNA (FIGS. 16A-16H; 14), however it was developed/optimized for DNAand uses a lysis/binding buffer consisting of GuHCI, GuSCN, ethanol, and detergents. Recently, it was shown that bacterial rRNA and mRNA recovery from whole blood and on silica matrices (like the TruTip) are improved by using a lysis/binding buffer composed solely of [4M GuSCN and 1% Tween-20] (16). Therefore, a 5-10-fold improvement in cLoD may be expected by optimizing OctaPlus lysis/binding buffer and elution conditions specifically for RNA. Other steps we can take to improve the estimated cLoD also may be improved by processing up to 8 mL of whole blood; using plasma instead of whole blood as the starting sample to simplify the extraction process, or lengthening the NASBA reaction to increase the amplification factor.

### Automation of Isothermal Amplification on the OctaPlus

The OctaPlus has two heated zones or strips so that one zone may be set at 65° C. (or higher) for primer annealing, and the other zone may be set to 41° C. (or higher) for the multiplex NASBA reaction. 96-well deep well reagent plates are pre-loaded with 20 μL of purified RNA, 10 μL of isothermal master mix, and 50 μL of concentrated hybridization buffer, and the plates are sealed with a foil seal. The plates are loaded onto the Octa deck, and sit at ambient room temperature for the duration of a mock sample preparation protocol (15-30 min). The program to run an automated, isothermal amplification is then initiated as per the methods described above. The experimental matrix is the same as described above, and encompasses 12 bacterial isolates, at least n=3 replicates, and a serial dilution of target RNA from 1 ng to 10 fg per reaction. A successful isothermal amplification is correct microarray-based detection of each target microorganism and drug resistance marker across all replicates at ≤0.1-1 pg total RNA per reaction (˜1-10 cell equivalents).

Under normal (manual) circumstances, a 1 mL pipette tip is accurate to ˜0.3% of the full pipette volume (17), or ±3 μL. But ±3 μL in a 30 μL total reaction volume is a 10% error. NASBA reactions are relatively robust to fluctuations in reactant concentrations because the amplification reaction is driven by the T7 polymerase, not the primers. While it is known in the art that automated pipetting is more precise than manual pipetting, Octa pipetting errors might generate a larger microarray standard deviation or lower LoD relative to manual methods. In addition to those contingencies listed in the isothermal master mix section, another option is to increase the volume of purified RNA and master mix, thus reducing the relative volumetric error. A second possible error is related to water evaporation in an “open” plate at 41° C., which may change NASBA reactant concentrations. If adverse effects are observed, then one solution is to add a droplet of mineral oil to the master mix “column” during plate manufacture, so that the isothermal multiplex ultimately occurs under the oil overlay and does not reflux during the 41° C. incubation step. Any residual mineral oil should be removed in subsequent steps, and not interfere with microarray hybridization or washing.

### Integration of Microarray Hybridization and Washing into the OctaPlus

The last automation step is to add concentrated hybridization buffer (containing universal, phycoerythrin-labeled detector probes) and wash buffer to the reagent plate, and perform an on-board microarray hybridization and wash. The finished product (plate) is organized as in FIG. 18. The user places the plate onto the OctaPlus platform, adds 0.4 mL of TruTip lysis buffer to each 0.2 mL blood sample, and places those samples in heater strip H1. Once samples are on the deck, the user starts the run. Once started, samples are lysed at ˜56° C. for 10 min, then mixed with 95% ethanol in column 1. RNA is captured by the TruTip matrix by 10 cycles of aspiration+dispense, and washed with 5 pipetting cycles in wash buffer 1 and 2, respectively. Pipette tips are centered over the waste reservoirs in column 6, and the TruTip matrix “dried” by a stream of clean air. TruTips then pick up ˜100 μL of elution buffer from column 4, move to column 5, and elute the purified RNA with 5-10 successive pipetting cycles. As RNA is being purified, the system turns on heater strip H2 and holds it at 65° C. Once purified RNA is in column 5, the TruTips pick up ˜40 μL of primer/buffer mix from column 12 and transfer it to the pre-heated tubes in heater strip H2. Next, the TruTips pick up ˜20 L of purified RNA from column 5, and mix it with the primer/buffer mix in H2. The annealing reaction proceeds for 5 minutes, at which point the temperature for H2 is reduced to 41° C. As the heater drops to 41° C., the entire contents of H2 (˜60 μL) is transferred and mixed with the NASBA enzymes and nucleotides in column 11. Then, the entire NASBA reaction (˜100 μL) is transferred back to heater strip H2 at 41° C. for 30 min−1 hr.

During the amplification step, the OctaPlus prepares microarrays for hybridization. First, TruTips are rinsed with residual elution buffer from column 4. Next, 0.2 mL of water is taken from column 7, pipetted onto the microarray, and immediately the water wash is pipetted back into the waste reservoirs in column 6. The water wash step is repeated once more, except the water sits on top of the microarray for 5 minutes before removing it to waste. Next, 0.2 mL of pre-hybridization solution from column 10 is added to the arrays, and after 5 minutes it is pipetted back to the waste reservoirs in column 6. Finally, 0.3 mL of hybridization solution from column 9 is added to the microarrays where it sits on the microarrays until the isothermal amplification is finished.

Once the isothermal amplification reaction is finished, the system takes the entire NASBA reaction (˜100 μL) from H2, adds it directly to its corresponding microarray well that already has hybridization buffer on the arrays, mixes by pipetting up and down (in 100 μL strokes), and then incubates the array for 30 min at room temperature. The TruTips are used to slowly pipette back and forth over the microarray, thus introducing active mixing into the hybridization step. After the 30 min hybridization step, the system removes the hybridization reaction to the column 6 waste reservoirs, rinses the pipette tips with pre-hybridization solution from column 10, and washes the arrays 4×0.2 mL of wash buffer from column 8, removing all wash solutions to the waste reservoirs in column 6. At this point, the run is finished. Arrays may sit at ambient room temperature until they are removed from the OctaPlus, and imaged on the Sensovation imager.

### Verification of Automated System Efficacy with Contrived Blood Samples

Reagents are pre-loaded into the plate, the plate is sealed and the complete, sample-to-answer test is run using whole blood that is spiked with the 12 ESKAPE+ pathogens, as described above, in a serial dilution to achieve final concentrations of 1,000, 300, 100, 10, 1, and 0 CFU/mL. At least 10 replicate samples of at least 1 mL each are processed. Verification of system efficacy is correct microarray detection of each target microorganism and drug resistance marker across all 10 replicates with no false positive signals on non-target probes, and an estimated clinical LoD between 1-10 CFU/mL whole blood.

### Cross-Contamination Study

The OctaPlus system is “open”, which raises a question of possible cross-contamination between wells, and between runs on the same instrument. A cross-contamination study is performed to demonstrate that the “open” OctaPlus does not introduce a nucleic acid contamination risk for the user. 1 μg of A. bauminnii total RNA (˜107 cell equivalents in 0.2 mL) is placed into every other position in the first column of the 96-well deep well plate (Column 1, Rows A, C, E, and G). The remaining wells (Column 1, Rows B, D, F, and H) are filled with 0.2 mL ultra-pure water, and then the plate is processed with the sample-to-answer procedure as in the hybridization and washing integration described above. In the next run, the position of A. bauminnii RNA and water are switched. These two steps are alternated for at least 40 runs, so that each row and pipettor is challenged with 20 replicate “exposures” to 1 μg total RNA and the resulting amplicons.

A 100% detection where A. bauminnii RNA is present and zero detectable A. bauminnii signal from all water blanks, across all runs demonstrates no cross-contamination. Although not expected, it is possible that pipetting amplified transcripts may cause some cross-contamination, especially across runs. In this case, the reagent plate may be reconfigured to include an RNA “zap” solution ahead of the microarray wash step. Separately, the OctaPlus may also be fitted with an interior UV light, which cross-links or degrades any residual nucleic acids on exposed surfaces.

### Updated Augury Software

Updating the Augury software enables correct microarray detection and reporting for each target microorganism and drug resistance marker. The microarray map and data analysis rules and the test output/report are encoded into the existing Augury software. The background fluorescence (or Limit of Blank) from contrived clinical samples establishes the SNR threshold for the data analysis algorithm. Analysis scripts are written in C++. In general, the decision and reporting tree answers the following questions:
1. Is the human beta 2 microglobulin (B2M) internal positive control detected over background? If NO, then indicate to the user that the test is Invalid—no other information should be reported to the user. If YES, then indicate to the user that the test is VALID, and proceed to step 2.
2. Is there a positive signal on any of the 16S-targeted probes? If NO, then terminate the analysis and indicate to the user that the target organisms were not detected. If YES, proceed with drug resistance determinants and generate an antimicrobial resistance profile.

The reporting rules for microbial identification require that all species-specific 16S rRNA probes (at least 6 probes per species, representing the H3 and H6 hypervariable regions; Table 13) are detected above their predetermined SNR values, as described above, otherwise, the species is Not Detected.

### REFERENCES

1. Antibiotic Resistance Threats in the United States, U.S. Department of Health and Human Services, CDC, Atlanta, GA (2019).
2. Santajit, S. and Indrawattana, N., Biomed. Res. Int., 247506 (2016).
3. Patel et al. Front. Microbiol. 7 (2016).
4. Mendes et al. J. Clin. Microbiol. 45:544-7 (2007).
5. Lund et al. Diagn. Microbiol. Infect. Dis. 92, 8-12 (2018).
6. Brown-Jaque et al. Front. Microbiol. 9:856 (2018).
7. Colomer-Lluch et al. PLOS One, 6: e17549 (2011).
8. Kelley et al. J. Clin. Microbiol. 51:2033-9 (2013).
9. He et al. J. Microbiol. Immunol. Infect. 53:746-750, 2020.
10. Li et al. Front. Microbiol. 8, 2078 (2017).
11. Zimmerman et al. J. Microbiol. Methods, 91:366-376 (2012).
12. Katchman et al. J. AOAC Int. 105:1390-1407 (2022).
13. Katchman et al. J. AOAC Int. 105:1105-1125 (2022).
14. Beall et al. PLOS One, 14: e0215753 (2019).
15. Brennecke et al. J. Med. Microbiol. 66:301-311 (2017).
16. Zainabadi et al. Sci. Rep. 12:17382 (2022).
17. Lippi et al. Clin. Chem. Lab. Med. 55:962-966 (2017).
18. Scheler et al. BMC Biotechnol, 9:45 (2009 May 15).
19. Scheler et al. BMC Biotechnol. 11:17 (2011).
20. van Gemen et al. J Virol Methods, 49 (2): 157-167 (1994).
21. Gill et al. Biochemical and Biophysical Research Communications, 347 (4): 1151-1157 (2006).
22. Ghalami M and Tehrani HA. Biochemical and Biophysical Research Communications, 347 (4): 1151-1157 (2006).
23. Morisset et al. Nucl Acids Res, 36 (18): e118 (2008).
24. Yan et al. Mol Biosyst. 10 (5): 970-1003 (2014 May).
25. Asiello, PJ and Baeumner, AJ. Lab Chip, 11 (8): 1420-30 (2011 Apr. 21).
26. Morisset et al. Nucleic acids research, 36: e118 (2008).
27. Mader et al. Analytical and bioanalytical chemistry, 397:3533-3541 (2010).
28. Scheler et al. BMC biotechnology, 9:45 (2009).
29. Kaplinski et al. BMC biotechnology, 10:34 (2010).
30. Jauset-Rubio et al. Sci Rep 6, 37732 (2016).

What is claimed is:

1. A method for identifying antibiotic resistance in a subject in need thereof, comprising:
obtaining a whole blood sample from the subject;
extracting RNA from the whole blood sample;
performing, on the RNA, a multiplexed, isothermal nucleic acid sequence-based amplification (NASBA) reaction to generate a plurality of RNA amplicons using at least one forward isothermal primer and at least one reverse isothermal primer each comprising nucleotide sequences specific to an antibiotic resistance determinant in a blood-borne pathogenic bacteria and specific to a detector probe binding site and on each reverse isothermal primer a T7 promoter sequence;
hybridizing, at room temperature, the plurality of RNA amplicons to a plurality of labeled universal detector probes and to a plurality of hybridization probes each comprising a nucleotide sequence complementary to the nucleotide sequence specific to the antibiotic resistance determinant in the blood-borne pathogenic bacteria; said hybridization probes attached to a solid substrate on a microarray via an oligonucleotide linker to form a three dimensional structure thereon;
washing the microarray at least once; and
imaging the microarray to detect at least one signal from at least one of the plurality of labeled universal detector probes, thereby identifying antibiotic resistance in the subject.

2. The method of claim 1, wherein the blood-borne pathogenic bacteria are Enterococcus faecium, Staphylococcus aureus, Klebsiella pneumoniae, Acinetobacter baumannii, Pseudomonas aeruginosa, Enterobacter cloacae, Enterobacter asburiae, Enterobacter hormaechei, Escherichia coli, or Klebsiella aerogenes.

3. The method of claim 1, wherein the isothermal primer pairs target a 16S rRNA hypervariable 3 region or a 16S rRNA hypervariable 6 region or a combination thereof.

4. The method of claim 3, wherein the forward isothermal primer and the reverse isothermal primer targeting the 16S rRNA hypervariable 3 (HV3) region comprise nucleotide sequences of SEQ ID NO: 117-118 or the forward isothermal primer and the reverse isothermal primer targeting the 16S rRNA hypervariable 6 (HV6) region comprise nucleotide sequences of SEQ ID NO: 119-120.

5. The method of claim 1, wherein the antibiotic resistance determinant in the blood-borne pathogenic bacteria is an imp-1 gene, a kpc gene, an oxa-48 gene, a ndm-1 gene, a vim gene, a mcr-1 gene, a ctx-m gene, a mecA gene, a mecC gene, a vanA gene, or a vanB gene.

6. The method of claim 1, wherein the forward isothermal primer and the reverse isothermal primer amplifying the specific antibiotic resistance determinant sequence comprise at least one of the group consisting of SEQ ID NOS: 121-122, SEQ ID NOS: 123-124, SEQ ID NOS: 125-126, SEQ ID NOS: 127-128, SEQ ID NOS: 129-130, SEQ ID NOS: 131-132, SEQ ID NOS: 133-134, SEQ ID NOS: 135-136, SEQ ID NOS: 137-138, SEQ ID NOS: 139-140, and SEQ ID NOS: 141-142.

7. The method of claim 1, wherein the plurality of hybridization probes comprise at least one nucleotide sequence from each of an HV3a region, an HV3c region, an HV3e region, an HV6a region, an HV6c region and an HV6g region and at least one nucleotide sequence from the antibiotic resistance determinants.

8. The method of claim 7, wherein the at least one nucleotide sequence from the HV3a region is selected from the group consisting of SEQ ID NOS: 145 to 156; wherein the at least one nucleotide sequence from the HV3c region is selected from the group consisting of SEQ ID NOS: 157 to 165; wherein the at least one nucleotide sequence from the HV3e region is selected from the group consisting of SEQ ID NOS: 166 to 170; wherein the at least one nucleotide sequence from the HV6a region is selected from the group consisting of SEQ ID NOS: 171 to 176; wherein the at least one nucleotide sequence from the HV6c region is selected from the group consisting of SEQ ID NOS: 177 to 185; wherein the at least one nucleotide sequence from the HV6g region is selected from the group consisting of SEQ ID NOS: 186 to 192; and wherein the at least one nucleotide sequence from the antibiotic resistance determinants is selected from the group consisting of SEQ ID NOS: 195 to 206.

9. The method of claim 1, wherein the labeled universal detector probes comprise a fluorescent label.

10. The method of claim 1, wherein said method is performed with a clinical level of detection of 1 CFU/ml to about 10 CFU/ml.

11. A method for detecting drug resistance in a blood-borne pathogenic bacteria, comprising:
obtaining a whole blood sample from a subject with an infection caused by the blood-borne pathogenic bacteria;
extracting RNA from the whole blood sample;
performing, on the RNA, a multiplexed, isothermal nucleic acid sequence-based amplification (NASBA) reaction to generate a plurality of RNA amplicons using at least one isothermal primer pair, said isothermal primer pairs comprising a forward isothermal primer and a reverse isothermal primer each of which comprise nucleotide sequences specific to a drug resistance marker in the blood borne pathogenic bacteria and to a detector probe binding site, each of said reverse isothermal primer further comprising a T7 promoter sequence;
hybridizing, at room temperature, the plurality of RNA amplicons to a plurality of fluorescent labeled universal detector probes and to a plurality of hybridization probes each comprising a nucleotide sequence complementary to the nucleotide sequence specific to the antibiotic resistance genetic marker; said hybridization probes attached to a solid substrate on a microarray via an oligonucleotide linker to form a three dimensional structure thereon;
washing the microarray at room temperature at least once; and
imaging the microarray to detect at least one signal from at least one of the plurality of labeled universal detector probes, thereby detecting drug resistance in the blood borne pathogenic bacteria.

12. The method of claim 11, wherein the blood-borne pathogenic bacteria are Enterococcus faecium, Staphylococcus aureus, Klebsiella pneumoniae, Acinetobacter baumannii, Pseudomonas aeruginosa, Enterobacter cloacae, Enterobacter asburiae, Enterobacter hormaechei, Escherichia coli, or Klebsiella aerogenes.

13. The method of claim 11, wherein the drug resistance marker is an imp-1 gene, a kpc gene, an oxa-48 gene, a ndm-1 gene, a vim gene, a mcr-1 gene, a ctx-m gene, a mecA gene, a mecC gene, a vanA gene, or a vanB gene.

14. The method of claim 13, wherein the imp-1 gene, the ndm-1 gene, the kpc gene, and the ctx-m gene and the kpc gene, the oxa-48 gene, the vim gene, the mcr-1 gene, the ctx-m gene are markers for resistance to carbapenem in Acinetobacter and in Enterobacteriaceae, respectively; wherein the vanA gene and the vanB gene are markers for resistance to vancomycin in Enterococci; wherein the mecA gene and the mecC gene are markers for resistance to methicillin in Staphylococcus aureus; wherein the kpc gene, the vim gene and the ctx-m gene are markers for multi-drug resistance in Pseudomonas aeruginosa; and wherein the ctx-m gene and the mcr-1 gene are markers for drug resistance in extended spectrum beta-lactamase (ESBL) producing Enterobacteriaceae.

15. The method of claim 11, wherein the isothermal primer pairs target a 16S rRNA hypervariable 3 region or a 16S rRNA hypervariable 6 region or a combination thereof.

16. The method of claim 15, wherein the isothermal primer pair targeting the 16S rRNA hypervariable 3 (HV3) region comprises nucleotide sequences of SEQ ID NO: 117-118 or the isothermal primer pair targeting the 16S rRNA hypervariable 6 (HV6) region comprise nucleotide sequences of SEQ ID NO: 119-120.

17. The method of claim 11, wherein the isothermal primer pairs amplifying the specific drug resistance marker sequence comprise at least one of the group consisting of SEQ ID NOS: 121-122, SEQ ID NOS: 123-124, SEQ ID NOS: 125-126, SEQ ID NOS:
127-128, SEQ ID NOS: 129-130, SEQ ID NOS: 131-132, SEQ ID NOS: 133-134, SEQ ID NOS: 135-136, SEQ ID NOS: 137-138, SEQ ID NOS: 139-140, and SEQ ID NOS: 141-142.

18. The method of claim 11, wherein the plurality of hybridization probes comprise at least one nucleotide sequence from each of an HV3a region, an HV3c region, an HV3e region, an HV6a region, an HV6c region and an HV6g region and at least one nucleotide sequence from the drug resistance markers.

19. The method of claim 18, wherein the at least one nucleotide sequence from the HV3a region is selected from the group consisting of SEQ ID NOS: 145 to 156; wherein the at least one nucleotide sequence from the HV3c region is selected from the group consisting of SEQ ID NOS: 157 to 165; wherein the at least one nucleotide sequence from the HV3e region is selected from the group consisting of SEQ ID NOS: 166 to 170; wherein the at least one nucleotide sequence from the HV6a region is selected from the group consisting of SEQ ID NOS: 171 to 176; wherein the at least one nucleotide sequence from the HV6c region is selected from the group consisting of SEQ ID NOS: 177 to 185; wherein the at least one nucleotide sequence from the HV6g region is selected from the group consisting of SEQ ID NOS: 186 to 192; and wherein the at least one nucleotide sequence from the drug resistance markers is selected from the group consisting of SEQ ID NOS: 195 to 206.

20. The method of claim 11, wherein said method is performed with a clinical level of detection of 1 CFU/ml to about 10 CFU/ml.
